(19) United States
(12) Patent Application Publication
(10) Pub. No.: US 2006/0172920 A1
(43) Pub. Date: Aug. 3, 2006

(54) Oxidised lipids as reversal agents for boronic acid drugs

(76) Inventors: Michael Finbarr Scully, London (GB); Christopher Andrew Goodwin, London (GB); John Joseph Deadman, Melbourne (AU)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/292,302

(22) Filed: Nov. 30, 2005

Related U.S. Application Data
(60) Provisional application No. 60/632,786, filed on Dec. 2, 2004.

(30) Foreign Application Priority Data
Nov. 30, 2004 (GB) 0426265.5

(51) Int. Cl. A61K 38/17; A61K 31/22; A61K 31/202

(52) U.S. Cl. 514/2; 514/547; 514/560

(57) ABSTRACT

The use of a lipid in oxidised form for the manufacture of a medicament for therapeutically neutralising (i.e. reducing or substantially destroying the activity of) an organoboronate drug. The lipid may be an HDL hydroperoxide. The drug may be TRI 50c or a salt or prodrug thereof.

### CROSS-REFERENCE

This application claims the benefit of Great Britain Patent Application No. 0426265.5, filed on Nov. 30, 2004, and U.S. Provisional Application No. 60/632,786, filed Dec. 2, 2004, which applications are incorporated herein by reference in their entirety.

### BACKGROUND

The present disclosure relates to inhibitors of biologically active organoboronates and more particularly of organoboronate medicaments and enzyme inhibitors; the enzymes are more particularly serine proteases, e.g. serine protease anticoagulants. The disclosure additionally includes activated lipids. The disclosure also relates to the use of the aforesaid products, to their formulation, and to other subject matter.

Boronic Acid Compounds

It has been known for some years that boronic acid compounds and their derivatives, e.g. esters, have biological activities, notably as inhibitors or substrates of proteases. For example, Koehler et al. Biochemistry 10:2477, 1971 report that 2-phenylethane boronic acid inhibits the serine protease chymotrypsin at millimolar levels. The inhibition of chymotrypsin and subtilisin by arylboronic acids (phenylboronic acid, m-nitro-phenylboronic acid, m-aminophenylboronic acid, m-bromophenylboronic acid) is reported by Phillip et al, Proc. Nat. Acad. Sci. USA 68:478-480, 1971. A study of the inhibition of subtilisin Carlsberg by a variety of boronic acids, especially phenyl boronic acids substituted by Cl, Br, CH₃, H₂N, MeO and others, is described by Seufer-Wasserthal et al, Biorg. Med. Chem. 2(1):35-48, 1994.

In describing inhibitors or substrates of proteases, P1, P2, P3, etc. designate substrate or inhibitor residues which are amino-terminal to the scissile peptide bond, and S1, S2, S3, etc., designate the corresponding subsites of the cognate protease in accordance with: Schechter, I. and Berger, A. On the Size of the Active Site in Proteases, Biochem. Biophys. Res. Comm., 27:157-162, 1967. In thrombin, the S1 binding site or “specificity pocket” is a well defined groove in the enzyme, whilst the S2 and S3 binding subsites (also respectively called the proximal and distal hydrophobic pockets) are hydrophobic and interact strongly with, respectively, Pro and (R)-Phe, amongst others.

Pharmaceutical research into serine protease inhibitors has moved from the simple arylboronic acids to boropeptides, i.e. peptides containing a boronic acid analogue of an α-amino carboxylic acid. The boronic acid may be derivatised, often to form an ester. Shenvi (EP-A-145441 and U.S. Pat. No. 4,499,082) disclosed that peptides containing an α-aminoboronic acid with a neutral side chain were effective inhibitors of elastase and has been followed by numerous patent publications relating to boropeptide inhibitors of serine proteases. Specific, tight binding boronic acid inhibitors have been reported for elastase (Kᵢ, 0.25 nM), chymotrypsin (Kᵢ, 0.25 nM), cathepsin G (Kᵢ, 21 nM), α-lytic protease (Kᵢ, 0.25 nM), dipeptidyl aminopeptidase type IV (Kᵢ, 16 pM) and more recently thrombin (Ac-D-Phe-Pro-boroArg-OH (DuP 714 initial Kᵢ 1.2 nM).

Claeson et al (U.S. Pat. No. 5,574,014 and others) and Kakkar et al (WO 92/07869 and family members including U.S. Pat. No. 5,648,338) disclose thrombin inhibitors having a neutral C-terminal side chain, for example an alkyl or alkoxyalkyl side chain.

Modifications of the compounds described by Kakkar et al are included in WO 96/25427, directed to peptidyl serine protease inhibitors in which the P2-P1 natural peptide linkage is replaced by another linkage. As examples of non-natural peptide linkages may be mentioned —CO₂—, —CH₂O—, —NHCO—, —CHYCH₂—, —CH═CH—, —CO(CH₂)ₚCO— where p is 1, 2 or 3, —COCHY—, —CO₂—CH₂NH—, —CHY—NX—, —N(X)CH₂—N(X)CO—, —CH═C(CN)CO—, —CH(OH)—NH—, —CH(CN)—NH—, —CH(OH)—CH₂— or —NH—CHOH—, where X is H or an amino protecting group and Y is H or halogen, especially F. Particular non-natural peptide linkages are —CO₂— or —CH₂O—.

Metternich (EP 471651 and U.S. Pat. No. 5,288,707) discloses variants of Phe-Pro-BoroArg boropeptides in which the P3 Phe is replaced by an unnatural hydrophobic amino acid such as trimethylsilylalanine, p-tert.butyl-diphenyl-silyloxymethyl-phenylalanine or p-hydroxymethyl-phenylalanine and the P1 side chain may be neutral (alkoxyalkyl, alkylthioalkyl or trimethylsilylalkyl).

The replacement of the P2 Pro residue of borotripeptide thrombin inhibitors by an N-substituted glycine is described in Fevig J M et al Bioorg. Med. Chem. 8: 301-306 and Rupin A et al Thromb. Haemost. 78(4):1221-1227, 1997. See also U.S. Pat. No. 5,585,360 (de Nanteuil et al).

Amparo (WO 96/20698 and family members including U.S. Pat. No. 5,698,538) discloses peptidomimetics of the structure Aryl-linker-Boro(Aa), where Boro(Aa) may be an aminoboronate residue with a non-basic side chain, for example BoroMpg. The linker is of the formula —(CH₂)ₘCONR— (where m is 0 to 8 and R is H or certain organic groups) or analogues thereof in which the peptide linkage —CONR— is replaced by —CSNR—, —SO₂NR—, —CO₂—, —C(S)O— or —SO₂O—. Aryl is phenyl, naphthyl or biphenyl substituted by one, two or three moieties selected from a specified group. Most typically these compounds are of the structure Aryl-(CH₂)ₙ—CONH—CHR²—BY¹Y², where R² is for example a neutral side chain as described above and n is 0 or 1.

Non-peptide boronates have been proposed as inhibitors of proteolytic enzymes in detergent compositions. WO 92/19707 and WO 95/12655 report that arylboronates can be used as inhibitors of proteolytic enzymes in detergent compositions. WO 92/19707 discloses compounds substituted meta to the boronate group by a hydrogen bonding group, especially acetamido (—NHCOCH₃), sufonamido (—NHSO₂CH₃) and alkylamino. WO 95/12655 teaches that ortho-substituted compounds are superior.

Boronate enzyme inhibitors have wide application, from detergents to bacterial sporulation inhibitors to pharmaceuticals. In the pharmaceutical field, there is patent literature describing boronate inhibitors of serine proteases, for example thrombin, factor Xa, kallikrein, elastase, plasmin as well as other serine proteases like prolyl endopeptidase and Ig AI Protease. Thrombin is the last protease in the coagulation pathway and acts to hydrolyse four small peptides from each molecule of fibrinogen, thus deprotecting its polymerisation sites. Once formed, the linear fibrin polymers may be cross-linked by factor XIIIa, which is itself activated by thrombin. In addition, thrombin is a potent activator of platelets, upon which it acts at specific receptors. Thrombin also potentiates its own production by the activation of factors V and VIII.

Other aminoboronate or peptidoboronate inhibitors or substrates of serine proteases are described in:

U.S. Pat. No. 4,935,493

EP 341661

WO 94/25049

WO 95/09859

WO 96/12499

WO 96/20689

Lee S-L et al, Biochemistry 36:13180-13186, 1997

Dominguez C et al, Bioorg. Med. Chem. Lett. 7:79-84, 1997

EP 471651

WO 94/20526

WO 95/20603

WO97/05161

U.S. Pat. No. 4,450,105

U.S. Pat. No. 5,106,948

U.S. Pat. No. 5,169,841.

Peptide boronic acid inhibitors of hepatic C virus protease are described in WO 01/02424. Matteson D S Chem. Rev. 89: 1535-1551, 1989 reviews the use of α-halo boronic esters as intermediates for the synthesis of inter alia amino boronic acids and their derivatives. Matteson describes the use of pinacol boronic esters in non-chiral synthesis and the use of pinanediol boronic esters for chiral control, including in the synthesis of amino and amido boronate esters.

Boronic acid and ester compounds have displayed promise as inhibitors of the proteasome, a multicatalytic protease responsible for the majority of intracellular protein turnover. Ciechanover, Cell, 79:13-21, 1994, teaches that the proteasome is the proteolytic component of the ubiquitin-proteasome pathway, in which proteins are targeted for degradation by conjugation to multiple molecules of ubiquitin. Ciechanover also teaches that the ubiquitin-proteasome pathway plays a key role in a variety of important physiological processes.

Adams et al, U.S. Pat. No. 5,780,454 (1998), U.S. Pat. No. 6,066,730 (2000), U.S. Pat. No. 6,083,903 (2000) and equivalent WO 96/13266, and U.S. Pat. No. 6,297,217 (2001) describe peptide boronic ester and acid compounds useful as proteasome inhibitors. These documents also describe the use of boronic ester and acid compounds to reduce the rate of muscle protein degradation, to reduce the activity of NF-κB in a cell, to reduce the rate of degradation of p53 protein in a cell, to inhibit cyclin degradation in a cell, to inhibit the growth of a cancer cell, to inhibit antigen presentation in a cell, to inhibit NF-κB dependent cell adhesion, and to inhibit HIV replication. Brand et al, WO 98/35691, teaches that proteasome inhibitors, including boronic acid compounds, are useful for treating infarcts such as occur during stroke or myocardial infarction. Elliott et al, WO 99/15183, teaches that proteasome inhibitors are useful for treating inflammatory and autoimmune diseases.

A proteasome inhibitor disclosed in the Adams et al patents is bortezomib (Velcade®), the compound N-(2-pyrazine)-carbonyl-phenylalanine-leucine-boronic acid.

WO 02/059131 discloses boronic acid products which are certain boropeptides and/or boropeptidomimetics in which the boronic acid group has been derivatised with a sugar. The disclosed sugar derivatives, which have hydrophobic amino acid side chains, are of the formula 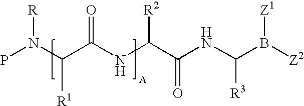
wherein:

P is hydrogen or an amino-group protecting moiety;

R is hydrogen or alkyl;

A is 0, 1 or 2;

R¹, R² and R³ are independently hydrogen, alkyl, cycloalkyl, aryl or —CH₂—R⁵;

R⁵, in each instance, is one of aryl, aralkyl, alkaryl, cycloalkyl, heterocyclyl, heteroaryl, or —W—R⁶, where W is a chalcogen and R⁶ is alkyl;

where the ring portion of any of said aryl, aralkyl, alkaryl, cycloalkyl, heterocyclyl, or heteroaryl in R¹, R², R³ or R⁵ can be optionally substituted; and

Z¹ and Z² together form a moiety derived from a sugar, wherein the atom attached to boron in each case is an oxygen atom.

Some of the disclosed compounds are sugar derivatives of bortezomib (see above), e.g. its mannitol ester.

Thrombosis

Hemostasis is the normal physiological condition of blood in which its components exist in dynamic equilibrium. When the equilibrium is disturbed, for instance following injury to a blood vessel, certain biochemical pathways are triggered leading, in this example, to arrest of bleeding via clot formation (coagulation). Coagulation is a dynamic and complex process in which proteolytic enzymes such as thrombin play a key role. Blood coagulation may occur through either of two cascades of zymogen activations, the extrinsic and intrinsic pathways of the coagulation cascade. Factor VIIa in the extrinsic pathway, and Factor IXa in the intrinsic pathway are important determinants of the activation of factor X to factor Xa, which itself catalyzes the activation of prothrombin to thrombin, whilst thrombin in turn catalyses the polymerization of fibrinogen monomers to fibrin polymer. The last protease in each pathway is therefore thrombin, which acts to hydrolyze four small peptides (two FpA and two FpB) from each molecule of fibrinogen, thus deprotecting its polymerization sites. Once formed, the linear fibrin polymers may be cross-linked by factor XIIIa, which is itself activated by thrombin. In addition, thrombin is a potent activator of platelets, upon which it acts at specific receptors. Thrombin activation of platelets leads to aggregation of the cells and secretion of additional factors that further accelerate the creation of a hemostatic plug. Thrombin also potentiates its own production by the activation of factors V and VIII (see Hemker and Beguin in: Jolles, et. al., “Biology and Pathology of Platelet Vessel Wall Interactions,” pp. 219-26 (1986), Crawford and Scrutton in: Bloom and Thomas, “Haemostasis and Thrombosis,” pp. 47-77, (1987), Bevers, et. al., Eur. J. Biochem. 122:429-36, 1982, Mann, Trends Biochem. Sci. 12:229-33, 1987).

Proteases are enzymes which cleave proteins at specific peptide bonds. Cuypers et al., J. Biol. Chem. 257:7086, 1982, and the references cited therein, classify proteases on a mechanistic basis into five classes: serine, cysteinyl or thiol, acid or aspartyl, threonine and metalloproteases. Members of each class catalyse the hydrolysis of peptide bonds by a similar mechanism, have similar active site amino acid residues and are susceptible to class-specific inhibitors. For example, all serine proteases that have been characterised have an active site serine residue.

The coagulation proteases thrombin, factor Xa, factor VIIa, and factor IXa are serine proteases having trypsin-like specificity for the cleavage of sequence-specific Arg-Xxx peptide bonds. As with other serine proteases, the cleavage event begins with an attack of the active site serine on the scissile bond of the substrate, resulting in the formation of a tetrahedral intermediate. This is followed by collapse of the tetrahedral intermediate to form an acyl enzyme and release of the amino terminus of the cleaved sequence. Hydrolysis of the acyl enzyme then releases the carboxy terminus.

A thrombus can be considered as an abnormal product of a normal mechanism and can be defined as a mass or deposit formed from blood constituents on a surface of the cardiovascular system, for example of the heart or a blood vessel. Thrombosis can be regarded as the pathological condition wherein improper activity of the hemostatic mechanism results in intravascular thrombus formation.

The management of thrombosis commonly involves the use of antiplatelet drugs (inhibitors of platelet aggregation) to control future thrombogenesis and thrombolytic agents to lyse the newly formed clot, either or both such agents being used in conjunction or combination with anticoagulants. Anticoagulants are used also preventatively (prophylactically) in the treatment of patients thought susceptible to thrombosis.

Neutral P1 Residue Boropeptide Thrombin Inhibitors

Claeson et al (U.S. Pat. No. 5,574,014 and others) and Kakkar et al (WO 92/07869 and family members including U.S. Pat. No. 5,648,338) disclose lipophilic thrombin inhibitors having a neutral (uncharged) C-terminal (P1) side chain, for example an alkoxyalkyl side chain.

The Claeson et al and Kakkar et al patent families disclose boronate esters containing the amino acid sequence D-Phe-Pro-BoroMpg [(R)-Phe-Pro-BoroMpg], which are highly specific inhibitors of thrombin. Of these compounds may be mentioned in particular Cbz-(R)-Phe-Pro-BoroMpg-OPinacol (also known as TRI 50b). The corresponding free boronic acid is known as TRI 50c. For further information relating to TRI 50b and related compounds, the reader is referred to the following documents:
Elgendy S et al., in The Design of Synthetic Inhibitors of Thrombin, Claeson G et al Eds, Advances in Experimental Medicine, 340:173-178, 1993.
Claeson G et al, Biochem. 290:309-312, 1993
Tapparelli C et al, J Biol Chem, 268:4734-4741, 1993
Claeson G, in The Design of Synthetic Inhibitors of Thrombin, Claeson G et al Eds, Advances in Experimental Medicine, 340:83-91, 1993
Phillip et al, in The Design of Synthetic Inhibitors of Thrombin, Claeson G et al Eds, Advances in Experimental Medicine, 340:67-77, 1993
Tapparelli C et al, Trends Pharmacol. Sci 14:366-376, 1993
Claeson G, Blood Coagulation and Fibrinolysis 5:411-436, 1994
Elgendy et al, Tetrahedron 50:3803-3812, 1994
Deadman J et al, J Enzyme Inhibition 9:29-41, 1995
Deadman J et al, J Medicinal Chemistry 38:1511-1522, 1995.

The tripeptide sequence of TRI 50b has three chiral centres. The Phe residue is considered to be of (R)-configuration and the Pro residue of natural (S)-configuration, at least in compounds with commercially useful inhibitor activity; the Mpg residue is believed to be of (R)-configuration in isomers with commercially useful inhibitor activity. TRI 50b acts as a prodrug for corresponding free acid TRI 50c, which is the active principle. The active, or most active, TRI 50c stereoisomer is considered to be of (R,S,R)-configuration and may be represented as: 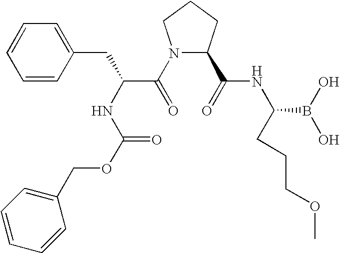 (R,S,R)-TRI 50c Cbz-(R)-Phe-(S)-Pro-(R)-Mpg-B(OH)₂

WO 2004/022072, and also U.S. Ser. No. 10/659,178 and EP-A-1396270, disclose pharmaceutically acceptable base addition salts of boronic acids which have a neutral aminoboronic acid residue capable of binding to the thrombin S1 subsite linked through a peptide linkage to a hydrophobic moiety capable of binding to the thrombin S2 and S3 subsites. In a first embodiment, there is disclosed a pharmaceutically acceptable base addition salt of a boronic acid of, for example, formula (A): 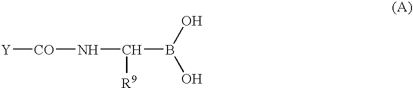
wherein

Y comprises a hydrophobic moiety which, together with the aminoboronic acid residue —NHCH(R⁹)—B(OH)₂, has affinity for the substrate binding site of thrombin; and

R⁹ is a straight chain alkyl group interrupted by one or more ether linkages (e.g. 1 or 2) and in which the total number of oxygen and carbon atoms is 3, 4, 5 or 6 (e.g. 5) or R⁹ is —(CH₂)ₘ—W where m is 2, 3, 4 or 5 (e.g. 4) and W is —OH or halogen (F, Cl, Br or I). R⁹ is an alkoxyalkyl group in one subset of compounds, e.g. alkoxyalkyl containing 4 carbon atoms. Salts of TRI 50c are exemplary.

WO 2004/022071, and also U.S. Ser. No. 10/659,179 and EP-A-1396269, disclose salts of a pharmaceutically acceptable multivalent (at least divalent) metal and an organoboronic acid drug. Such salts are described as having an improved level of stability which cannot be explained or predicted on the basis of known chemistry, and as being indicated to have unexpectedly high and consistent oral bioavailability not susceptible of explanation on the basis of known mechanisms. The oral formulations of such salts are therefore also disclosed.

One particular class of salts comprises those wherein the organoboronic acid comprises a boropeptide or boropeptidomimetic. Such drugs which may beneficially be prepared as salts include without limitation those of the formula X-(aa)ₙ-B(OH)₂, where X is H or an amino-protecting group, n is 2, 3 or 4, (especially 2 or 3) and each aa is independently a hydrophobic amino acid, whether natural or unnatural. In one class of multivalent metal salts, the organoboronic acid is of formula (A) above. Salts of TRI 50c are exemplary.

WO 2004/022070, and also U.S. Ser. No. 10/658,971 and EP-A-1400245, disclose and claim inter alia parenteral pharmaceutical formulations that include a pharmaceutically acceptable base addition salt of a boronic acid of, for example, formula (A) above. Such salts are described as having an improved level of stability which cannot be explained or predicted on the basis of known chemistry. Salts of TRI 50c are exemplary.

Non-Peptide Boronates

Non-peptide boronates have been proposed as inhibitors of proteolytic enzymes in detergent compositions. WO 92/19707 and WO 95/12655 report that arylboronates can be used as inhibitors of proteolytic enzymes in detergent compositions. WO 92/19707 discloses compounds substituted meta to the boronate group by a hydrogen bonding group, especially acetamido (—NHCOCH₃), sulfonamido (—NHSO₂CH₃) and alkylamino. WO 95/12655 teaches that ortho substituted compounds are superior.

Potential Bleeding Resulting from Use of Anticoagulants

When an anticoagulant is used as a therapeutic agent, there can be a requirement for rapid neutralisation should there be unexpected bleeding. The problem of unwanted bleeding is well recognised in the area of anticoagulant therapy; thus protamine is used to reverse the anticoagulant effect of heparin and vitamin K to reverse the effect of warfarin.

Lipids and Lipoproteins

Lipids may be classified as “complex” (saponifiable) which comprise fatty acids as building block components or “simple” (non-saponifiable) which contain no fatty acids. In addition, lipids may also exist in hybrid form as, for example, a lipoprotein which is a biomolecule with both lipid and protein moieties. There are three main classes of complex lipids: acylglycerols; phosphoglycerides; and sphingolipids. The three main classes of the simple lipids are: (i) terpenes, which are constructed of multiples of isoprene, for example squalene is a triterpene; (ii) sterols which originate from the linear triterpene squalene, which readily cyclises; and (iii) prostaglandins. Sterols comprise a large sub-group of steroids of which lanosterol and cholesterol are the principle members.

Cholesterol is insoluble in water, therefore it is transported in the blood and extracellular fluids conjugated to proteins, called apolipoproteins. This cholesterol and protein complex is known as lipoprotein. The lipoproteins are broadly classified into chylomicrons, very low density lipoprotein (VLDL), low density lipoprotein (LDL), intermediate density lipoprotein (IDL), and high density lipoprotein (HDL), based on their densities. The above groups also vary in their dimensions, cholesterol carrying-capacities, and their function.

LDLs are composed of a collection of spherical particles with an average diameter of 22 nanometers. The average LDL particle contains a hydrophobic core of 1500 molecules of cholesteryl ester surrounded by a polar coat composed primarily of phospholipids and a 513-kilodalton protein called apolipoprotein B-100 (apoB-100). LDLs are secreted from the liver as larger precursor particles (average diameter, 55 nanometers) called very low-density lipoproteins (VLDLs), whose cores contain triglycerides as well as cholesteryl esters. VLDL-triglycerides are removed in the capillaries of muscle and adipose tissue, and the particles then undergo exchange reactions with other lipoproteins. The net effect is to reduce the size of VLDLs, restricting the core lipids to cholesteryl esters, and removing all proteins except apoB-100, thereby producing LDLs. The unsaturated fatty acids of LDLs can undergo oxidation to generate lipid peroxides. In particular, lipid peroxides are formed when either free or bound polyunsaturated fatty acids are attacked by free radicals.

It is now well accepted that all plasma lipoproteins are composed of a hydrophobic core that is surrounded by an outer surface monolayer of phospholipids, cholesterol and the apoproteins, which are in an α-helical conformation.

Lipid hydroperoxides are the primary stable products of lipid peroxidation. For example, cholesteryl ester hydroperoxide (CE-OOH) and phosphatidylcholine hydroperoxide (PC—OOH) are formed as the major oxidation products when low density lipoproteins are exposed to oxygen radicals (Yamamoto, Y et al., Oxidative Damage and Repair, Davies, K. J. A. (editor). Pergamon Press: Oxford, pp 287-291; Stocker, R et al, Proc. Nat. Acad. Sci. USA 88, 1646-1650). The principal form of CE-OOH found in plasma is cholesteryl linoleate hydroperoxide. Typical chemical structures for some of these hydroperoxides are shown below:

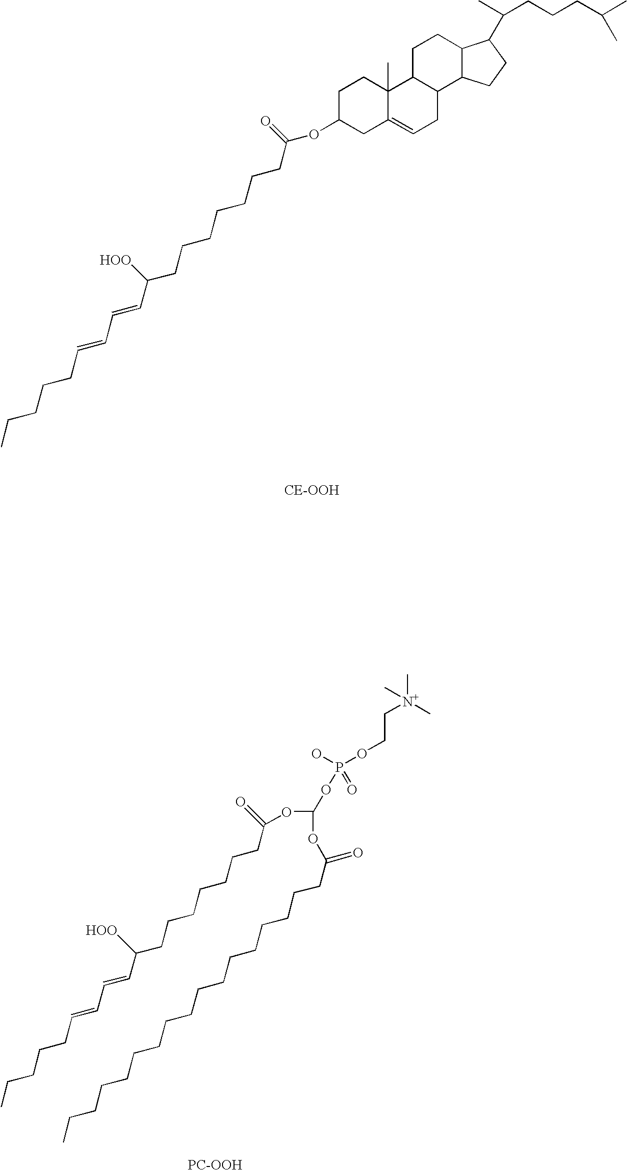

Whereas CE-OOH has been detected in human blood plasma, PC—OOH has not. It is likely that this is due to the presence of plasma glutathione peroxidase which can reduce PC—OOH but not CE-OOH, (Yamamoto Y. Fate of lipid hydroperoxides in blood plasma Free Radic. Res. 2000; 33: 795-800).

Representative of CE-OOH are cholesteryl linoleate hydroperoxides which are commercially available as a mixture of racemic 9- and 13-HpODE cholesteryl esters. Cholesteryl linoleate hydroperoxides are stable for at least six months if stored at −80° C. They are typically supplied as a solution in ethanol and sparingly soluble in aqueous buffers (<20 μg/ml in PBS pH 7.2), and further dilutions of stock solution into aqueous buffers or isotonic saline must be made prior to biological use.

Lipid peroxides are found in parenteral nutrition solutions.

### BRIEF SUMMARY OF THE DISCLOSURE

The present invention relates amongst other things to products useful for neutralising (reducing or destroying) the inhibitory activity of biologically active organoboronates. Unless the context otherwise requires, the term “boronate” as used herein includes reference to boronic acids as well as derivatised forms thereof, e.g. salts as well as esters and other prodrugs. In one embodiment, the present disclosure provides a product that can neutralise the activity of a boropeptide serine protease inhibitor and thus terminate or reduce its therapeutic effect if required. The term “neutralise” as used herein includes both reduction of activity and destruction of activity. In the pharmaceutical industry, compounds having such activities are known as “antidotes”, “reversal agents” and “neutralisers”, and doubtless by other names also.

Included in the disclosure is the use, for the manufacture of a medicament for therapeutically neutralising (e.g. reducing in activity) an organoboronate drug, of a lipid in oxidised form. Additionally included in the disclosure is the use, for the manufacture of a medicament for therapeutically neutralising (e.g. reducing in activity) an organoboronate drug, of a lipoprotein which has been treated with Cu (II).

In another aspect there is provided a lipid product, which is not active in fresh plasma but is generated by oxidation of plasma, the product being capable of neutralising the activity of an organoboronate and especially a boropeptide.

Reference herein to an organoboronate and especially a boropeptide is intended to include any species capable of being in equilibrium with an organoboronate, especially a peptide boronic acid, including boronate salts, boronic acids, derivatised or protected boronic acids, and complexes.

According to another aspect of the invention there is provided a lipid in oxidised form that is capable of neutralising an organoboronate and especially a boropeptidyl serine protease inhibitor and more particularly a boropeptide having the amino acid sequence Phe-Pro-BoroMpg, particularly (R)-Phe-(S)-Pro-(R)-BoroMpg.

The lipid is suitably unsaturated, e.g. contains an unsaturated aliphatic moiety, as in the case of a lipid comprising an unsaturated fatty acid.

Preferably, the oxidised lipid is in the form of a lipoprotein. In one aspect the lipid is an LDL. In another aspect, the lipid is an HDL. The invention therefore also includes an oxidised lipoprotein obtainable by, or having the characteristics of a product obtained by, oxidation of a lipoprotein.

According to further aspect of the invention there is provided a lipid in oxidised form obtainable by, or having the characteristics of a product obtained by, oxidation of a lipid in the presence of a source of Cu (II). The oxidised lipid is capable of neutralising an organoboronate and especially a boropeptidyl protease inhibitor. Reference herein to the presence of a source of Cu (II) is intended to mean that the level of Cu (II) is more than an ineffective trace amount.

Another aspect of the disclosure resides in a lipid peroxide obtainable by, or having the characteristics of a product obtained by, oxidation of lipid in the presence of a source of Cu (II). The subject matter of this application includes also a lipid peroxide for neutralising an organoboronate and especially a a boropeptidyl protease inhibitor, e.g. a boropeptidyl serine protease inhibitor. In addition the invention provides a lipid peroxide for use as a pharmaceutical.

According to a yet further aspect of the invention there is a pharmaceutical formulation comprising an oxidised lipid or oxidised lipoprotein, or a lipid peroxide, a sterol peroxide or a cholesteryl ester peroxide.

According to a yet further aspect of the invention there is provided use of an oxidised lipid or lipoprotein or lipid peroxide or a cholesteryl ester peroxide for the manufacture of a medicament for neutralising an organoboronate and especially a boropeptidyl serine protease inhibitor.

In embodiments, there is provided the use of an oxidised lipid or lipoprotein or lipid peroxide or a cholesteryl ester peroxide for the manufacture of a medicament for use in terminating or reducing the activity of a boropeptidyl serine protease inhibitor.

Additionally included is the use of an oxidised lipid or lipoprotein or lipid peroxide or a cholesteryl ester peroxide for the manufacture of a medicament is for use in treating bleeding resulting from the administration of a boronate, e.g. boropeptide, inhibitor of a coagulation serine protease, particularly thrombin.

One aspect of the disclosure resides in a method of neutralising an organoboronate, e.g. boropeptide, inhibitor of a protease, particularly of a serine protease, the method comprising the steps of contacting said organoboronate or boropeptidyl inhibitor with a product described herein. The method may be in vitro or ex vivo.

It is contemplated that oxidised lipid is not subject to enzymic degradation in plasma.

The present disclosure includes the subject matter of the following paragraphs:

1. The use, for the manufacture of a medicament for therapeutically neutralising (i.e. reducing or substantially destroying the activity of) an organoboronate drug, of a product produced by treating a lipid comprising an unsaturated aliphatic moiety with a source of Cu (II).

2. A method of neutralising a boropeptidyl serine protease inhibitor comprising contacting said boropeptidyl serine protease inhibitor with a product as defined herein, for example in any one of claims 1 to 14, or a formulation as defined herein, for example of any of claims 27 to 30.

3. An ex vivo method of neutralising a boropeptidyl serine protease inhibitor comprising contacting said boropeptidyl serine protease inhibitor with a product as described herein, e.g. as defined in any one of claims 1 to 14 or a formulation as described herein, e.g. of claims 27 to 30.

4. An in vitro method of neutralising a boropeptidyl serine protease inhibitor comprising contacting said boropeptidyl serine protease inhibitor with the product as described herein, e.g. as defined in any one of claims 1 to 14 or a formulation as described herein, e.g. of claims 27 to 30.

5. A method of production of a pharmaceutical composition for therapeutically neutralising an organoboronate drug, comprising combining the product as described herein, for example, according to any one of claims 1 to 14 with a pharmaceutically acceptable diluent, carrier or excipient.

6. A method of paragraph 5 wherein the composition is for therapeutically neutralising the activity of a serine protease inhibitor.

7. A method according to paragraph 6 wherein the serine protease inhibitor is a tripeptide having the amino acid sequence Phe-Pro-BoroMpg.

8. A method of treatment comprising administering a therapeutically effective amount of a product as described herein, e.g. as defined in any one of claims 1 to 14, or a formulation as described herein, e.g. of claims 27 to 30, to an individual who has received a boropeptidyl serine protease inhibitor so as to neutralise said inhibitor.

9. A method according to paragraph 8 wherein the product is administered in an amount such that there is a molar equivalence ratio of approximately 1:1 with boropeptidyl serine protease inhibitor in the individual's plasma.

10. A method of preparing to supply a first pharmaceutical composition for the treatment of thrombosis by prophylaxis or therapy and, if required, a second pharmaceutical composition to inhibit the action of the first composition, comprising

stocking a pharmaceutical composition comprising a pharmaceutically acceptable active compound which is capable of providing in the plasma a peptide boronic acid of formula (A); and

stocking a pharmaceutical formulation as described herein, e.g. according to any one of claims 27 to 30,
formula (A) being as follows: 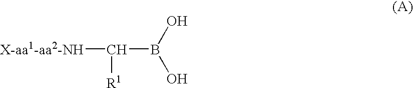
where:
X is H (to form NH₂) or an amino-protecting group;
aa¹ is Phe, Dpa or a wholly or partially hydrogenated analogue thereof;
aa² is an imino acid having from 4 to 6 ring members; and
R¹ is a group of the formula —(CH₂)ₘ—W, where m is 2, 3 or 4 and W is —OH, -OMe, -OEt or halogen (F, Cl, Br or I).

11. A method for treating thrombosis by prophylaxis or therapy using a medicament which results in inappropriate bleeding and then inhibiting the action of said medicament, wherein

a therapeutically effective amount of a pharmaceutical composition comprising a compound as recited in paragraph 10 is administered to a patient in need thereof to treat thrombosis, and, after the inappropriate bleeding,

a therapeutically effective amount of a product as described herein, e.g. in claims 1 to 14, or pharmaceutical formulation as described herein, e.g. according to any one of claims 27 to 30, is administered to the patient to inhibit the pharmaceutical composition.

12. A method for inhibiting thrombosis in the treatment of disease by prophylaxis or therapy using a medicament which results in inappropriate bleeding and then inhibiting the action of said medicament, wherein

a therapeutically effective amount of a pharmaceutical composition comprising a compound as recited in paragraph 10 is administered to a patient in need thereof to treat thrombosis, and, after the inappropriate bleeding,

a therapeutically effective amount of a product as described herein, e.g. of any of claims 1 to 14, or pharmaceutical formulation as described herein, e.g. according to any one of claims 27 to 30, is administered to the patient to inhibit the pharmaceutical composition comprising a compound as recited in paragraph 10.

13. A method according to any one of paragraphs 2 and 5 to 12 wherein the pharmaceutical composition and/or product are administered orally and/or intravenously.

14. Use, for the manufacture of a medicament pair comprising a first medicament for treating thrombosis by prophylaxis or therapy and a second medicament for, if required, stopping or reducing the anti-thrombotic treatment, of a compound as recited in paragraph 10 for the manufacture of the first medicament and a product as described herein, e.g. of any of claims 1 to 14 for the manufacture of the second medicament.

15. Use, for the manufacture of a medicament pair comprising a first medicament for treating thrombosis by prophylaxis or therapy and a second medicament for, if required, stopping or reducing undue or inappropriate bleeding caused by the first medicament comprising a compound as recited in paragraph 10 for the manufacture of the first medicament, and a product as described herein, e.g. of any of claims 1 to 14 for the manufacture of the second medicament.

16. A method of preparing for the administration to a patient of a first pharmaceutical composition for the treatment of thrombosis by prophylaxis or therapy and, if required, a second pharmaceutical composition for reacting with the active agent of the first composition to inactivate molecules thereof comprising

supplying a pharmaceutical composition comprising a compound as recited in paragraph 10, and supplying a pharmaceutical composition as described herein, e.g. according to any one of claims 27 to 30.

17. A method according to paragraph 16 wherein either or both of the first and second pharmaceutical compositions is for oral or intravenous administration.

18. A method according to any of paragraphs 10 to 17 wherein aa¹ is selected from Dpa, Phe, Dcha and Cha.

19. A method according to any of paragraphs 10 to 18 wherein aa¹ is an (R)-enantiomer.

20. A method according to paragraph 19 herein aa¹ is (R)-Phe or (R)-Dpa.

21. A method according to paragraph 20 wherein aa¹ is (R)-Phe.

22. A method according to any of paragraphs 10 to 21 wherein aa² is a residue of an imino acid, unrestricted as to enantiomer, of formula (III) 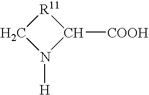
where R¹¹ is —CH₂—, CH₂—CH₂—, —S—CH₂—, —S—C(CH₃)₂— or —CH₂—CH₂—CH₂—, which group, when the ring is 5- or 6-membered, is optionally substituted at one or more —CH₂— groups by from 1 to 3 C₁-C₃ alkyl groups.

23. A method according to paragraph 22 wherein aa² is a natural proline residue.

24. A method according to any of paragraphs 10-23, wherein aa¹-aa² is (R)-Phe-(S)-Pro.

25. A method according to any of paragraphs 10 to 24 wherein R¹ is 2-bromoethyl, 2-chloroethyl, 2-methoxyethyl, 3-bromopropyl, 3-chloropropyl or 3-methoxypropyl.

26. A method according to any of paragraphs 10 to 25 wherein R¹ is 3-methoxypropyl.

27. A method according to any of paragraphs 10 to 26 wherein X-aa¹-aa²-NH—CHR¹B(OH)₂ is
X-D-Phe-Pro-Mpg-B(OH)₂.

28. A method according to paragraph 27 wherein the Mpg residue is of (R)-configuration.

29. A method according to any of paragraphs 10 to 28 wherein X is benzyloxycarbonyl.

30. A method according to any of paragraphs 10 to 29 wherein the compound of formula (I) is a pharmaceutically acceptable salt of a compound of formula (IV):
X—(R)-Phe-(S)-Pro-(R)-Mpg-B(OH)₂ (IV)

31. A method according to any of paragraphs 10 to 30 wherein the compound of formula (I) is a product which, after administration, releases a compound of formula (IV) and/or a corresponding peptide boronate anion:
X—(R)-Phe-(S)-Pro-(R)-Mpg-B(OH)₂ (IV)

32. A method for deboronating an organoboronate drug comprising treating the drug with an activated lipid.

33. A method of paragraph 32, wherein the organoboronate drug is one as described herein, for example as described in claims 15 to 21.

34. A method of paragraphs 32 or 33, wherein the lipid is as described herein, for example as defined in claims 1 to 14.

35. A method of any of paragraphs 32 to 34, wherein the degree of deboronation is directly related to the activity of the drug in the body.

36. A method of any one of paragraphs 32 to 35, wherein the greater the degree of deboronation the lower the therapeutic activity of the drug.

37. A method of forming the Impurity from TRI 50c or salts or esters thereof, for example TRI 50b, comprising treating TRI 50b, or salts or esters thereof with a lipid as described herein, for example in any one of claims 1 to 14.

38. A method of the preceding paragraph, wherein the impurity is formed in a mammal.

39. A method of the preceding two paragraphs, wherein the amount of impurity is directly related to the activity of the drug.

40. A method for forming a boron-containing lipid, comprising treating an organoboron compound with a lipid as described herein, for example in any of claims 1 to 14.

Throughout the description and claims of this specification, the words “comprise” and “contain” and variations of the words, for example “comprising” and “comprises”, mean “including but not limited to”, and are not intended to (and do not) exclude other moieties, additives, components, integers or steps.

As previously indicated, the term “neutralising” is used in this specification to refer to causing a reduction or loss of activity, e.g. serine protease activity.

### BRIEF DESCRIPTION OF THE DRAWINGS

FIG. 1 shows a graph of the effect of preincubation with Cu²⁺ ion on the ability of plasma to neutralise TRI-50b activity.

FIG. 2 shows graphs of the effect of time of incubation with Cu²⁺ ion at three different concentrations on the ability of plasma to neutralise TRI-50b activity.

FIG. 3 shows a graph of the separation of the TRI-50b neutralising activity by gel filtration.

FIG. 4 shows a graph of the effect of incubation with Cu²⁺ ion on the ability of preparation of purified lipoprotein to neutralise TRI-50b activity.

FIG. 5 shows a graph of the effect of time of incubation with Cu²⁺ ion on the ability of varying dilutions of purified lipoprotein to neutralise TRI-50b activity.

FIG. 6 shows a graph of protein content (OD 280) of fractions prepared from oxidised plasma by gel filtration.

FIG. 7 shows a graph of TRI-50b neutralising activity of fractions prepared from oxidised plasma.

FIG. 8 shows a graph of peroxide content of fractions (OD 550) of oxidised plasma prepared by gel filtration.

FIG. 9 shows a graph of the correlation between peroxide content of fractions of oxidised plasma and the neutralisation of TRI-50b.

FIG. 10 shows a graph of the determination of total neutralising capacity of a bovine lipoprotein fraction.

FIG. 11 shows a graph of the determination of total neutralising capacity of plasma following oxidation.

FIG. 12 shows a graph of the neutralisation of TRI-50b by cholesteryl linoleate hydroperoxide.

FIG. 13 shows a graph of the determination of the total neutralising capacity of bovine lipoprotein fraction towards different peptide boronate inhibitors.

FIG. 14 shows a graph of the effect of N-acetyl cysteine upon TRI-50b neutralising activity of bovine lipoprotein and oxidised plasma.

FIG. 15 shows a graph of the determination of the Ki of TRI-50b subjected to treatment with hydrogen peroxide.

FIG. 16 shows a graph of the neutralisation of TRI-50b in human plasma by the addition of bovine lipoprotein.

FIG. 17 shows the structure of a major product resulting from the oxidation of TRI 50b with H₂O₂.

FIG. 18 shows the effect of CuSO₄ on the inhibition of TRI 50c (1 μm).

FIG. 19 shows the effects of activated HDL on inhibition of TRI 50c.

FIG. 20 shows the effect of HDL on the inhibition of TRI 50c (1 μm).

FIG. 21 shows the effects of CuSO₄ on HDL activation and the inhibition of TRI 50c (1 μm).

FIG. 22 shows the effects of activated HDL (4.57 mg/ml final) on inhibition of TRI 50c.

FIG. 23 shows the activation time course with HDL inhibition of TRI 50c (5 μm).

FIG. 24 shows the effect of HDL on ECTs in human plasma.

FIG. 25 shows effects of oxidised HDL on ECT using fresh rat plasma.

FIG. 26 shows oxidised HDL stability study.

FIG. 27 shows in vivo administration of oxidised HDL to neutralise the anticoagulant activity of TGN 255 in rats.

FIG. 28 shows effect of oxidised HDL on TGN 255 infusion studies in rats.

FIG. 29 shows effect of oxidised HDL on TGN 255 infusion studies in rats.

FIG. 30 shows effect of oxidised HDL on TGN 255 infusion studies in rats.

FIG. 31 shows TGN 255 high dose rebound rat study.

FIG. 32 shows effects of intravenously administered oxidised HDL on group mean thrombin time (TT) in the anaesthetised rat.

FIG. 33 shows effects of intravenously administered oxidised HDL on group mean thrombin time (TT) in the anaesthetised rat.

FIG. 34 shows effects of intravenously administered oxidised HDL on group mean thrombin time (TT) in the conscious rat.

### DETAILED DESCRIPTION OF SEVERAL EXAMPLES

Glossary

The following terms and abbreviations are used in this specification: The term “aliphatic” refers to an open-chain or cyclic species not having aromatic properties. Such species may contain a combination of open-chain and cyclic parts. They may be saturated or unsaturated. Often “aliphatic” refers to open-chain species, whether linear or branched, linear being more common. Aliphatic species may be hydrocarbyl aliphatic. Aliphatic species are often substituted or unsubstituted alkyl, alkenyl or alkynyl; in many instances aliphatic is unsubstituted alkyl, alkenyl or alkynyl, e.g. is alkyl. The species may be a compound or part of a compound, as the context requires. Some aliphatic species contain from 1 to 15 in-chain or in-ring atoms, e.g. 1 to 10 such as 1 to 6, for example.

α-Aminoboronic acid or Boro(aa) refers to an amino acid in which the CO₂ group has been replaced by BO₂.

The term “amino-group protecting moiety” refers to any group used to derivatise an amino group, especially an N-terminal amino group of a peptide or amino acid. Such groups include, without limitation, alkyl, acyl, alkoxycarbonyl, aminocarbonyl, and sulfonyl moieties. However, the term “amino-group protecting moiety” is not intended to be limited to those particular protecting groups that are commonly employed in organic synthesis, nor is it intended to be limited to groups that are readily cleavable.

The term “coagulation serine protease” refers to a serine protease involved in the coagulation of blood, for example thrombin, Factor IXa or Factor X.

The phrase “pharmaceutically acceptable” is employed herein to refer to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio.

The expression “thrombin inhibitor” refers to a product which, within the scope of sound pharmacological judgement, is potentially or actually pharmaceutically useful as an inhibitor of thrombin, and includes reference to a substance which comprises a pharmaceutically active species and is described, promoted or authorised as a thrombin inhibitor. Such thrombin inhibitors may be selective, that is they are regarded, within the scope of sound pharmacological judgement, as selective towards thrombin in contrast to other proteases; the term “selective thrombin inhibitor” includes reference to a substance which comprises a pharmaceutically active species and is described, promoted or authorised as a selective thrombin inhibitor.

The term “heteroaryl” refers to a ring system which has at least one (e.g. 1, 2 or 3) in-ring heteroatoms and has a conjugated in-ring double bond system. The term “heteroatom” includes oxygen, sulfur and nitrogen, of which sulfur is sometimes less preferred.

“Natural amino acid” means an L-amino acid (or residue thereof) selected from the following group of neutral (hydrophobic or polar), positively charged and negatively charged amino acids:

Hydrophobic Amino Acids

A=Ala=alanine

V=Val=valine

I=Ile=isoleucine

L=Leu=leucine

M=Met=methionine

F=Phe=phenylalanine

P=Pro=proline

W=Trp=tryptophan

Polar (Neutral or Uncharged) Amino Acids

N=Asn=asparagine

C=Cys=cysteine

Q=Gln=glutamine

G=Gly=glycine

S=Ser=serine

T=Thr=threonine

Y=Tyr=tyrosine

Positively Charged (Basic) Amino Acids

R=Arg=arginine

H=His=histidine

K=Lys=lysine

Negatively Charged Amino Acids

D=Asp=aspartic acid

E=Glu=glutamic acid.

Amino acid=α-amino acid

Acid addition salt=a reaction product made by combining an inorganic acid or an organic acid with a free base of an active principle (e.g. an amino group).

Base addition salt=a reaction product made by combining an inorganic base or an organic base with a free acid (e.g. a carboxylic or boronic acid) of an active principle.

Cbz=benzyloxycarbonyl

Cha=cyclohexylalanine (a hydrophobic unnatural amino acid)

Charged (as applied to drugs or fragments of drug molecules, e.g. amino acid residues)=carrying a charge at physiological pH, as in the case of an amino, amidino or carboxy group

Dcha=dicyclohexylalanine (a hydrophobic unnatural amino acid)

Dpa=diphenylalanine (a hydrophobic unnatural amino acid)

Drug=a pharmaceutically useful substance, whether the active in vivo principle or a prodrug

i.v.=intravenous

Mpg=3-methoxypropylglycine (a hydrophobic unnatural amino acid)

Neutral (as applied to drugs or fragments of drug molecules, e.g. amino acid residues)=uncharged=not carrying a charge at physiological pH

Pinac=Pinacol=2,3-dimethyl-2,3-butanediol

Pinanediol=2,3-pinanediol=2,6,6-trimethylbicyclo[3.1.1]heptane-2,3-diol

Pip=pipecolinic acid

Room temperature=25° C.±2° C.

S2238=D-Phe-Pipecolyl-Arg-p-nitroanilide

s.c.=subcutaneous

THF=tetrahydrofuran

Thr=thrombin

VLDL=very low density lipoprotein

Initial encounter complex [EI] (also known as the Michaelis complex) refers to an interaction characterised by the equation:

E+I=EI→EI*, where E is the enzyme, I the inhibitor and EI* the final complex after slow-tight binding. Initial Ki refers to the formation of EI and final Ki (Ki*) to EI*.

The Products of the Disclosure

The present invention is predicated on the surprising observation that there was a loss of activity when an active boropeptide compound was incubated with certain preparations of human plasma. This loss of activity was not observed when the compound was incubated in fresh plasma preparations but was only observed when incubated with aged plasma, for example plasma that had been stored for approximately 6 months at −20° C. Further investigation revealed that the neutralising activity was associated with a number of plasma protein fractions but is principally derived from a lipid fraction of plasma and is particularly associated with an oxidised lipid fraction. Additional studies indicated that the destruction of the boropeptide occurs by interaction with lipid peroxide.

In one aspect, therefore, there is provided a lipid which is in oxidised form; the lipid has a neutralising effect on one or more boronates and especially a boronic acid inhibitor of a serine protease. In this context, reference to “boronic acid” includes salts and prodrugs of boronic acids; exemplary prodrugs are esters, particularly esters of diols such as, for example, pinacol, pinanediol and sugars. The boronates are discussed in more detail below under the heading “Target Compounds”.

Reference herein to “product” includes an oxidised lipid or oxidised lipoprotein or oxidised steroid or oxidised sterol; it includes reference also to lipid peroxide, lipoprotein peroxide, sterol peroxide or steroid peroxide. The peroxides may be hydroperoxides.

The present invention provides a product for controlling the activity of an organoboronates and especially a peptide boronic acid (which boronic acid may be presented in any form which releases the acid or a corresponding boronate ion after administration, e.g. in the form of a base addition salt or in protected form as a prodrug).

One class of products is obtainable by (i.e. has the characteristics of a product obtained by) treatment of lipid with a source of Cu (II). Desirably the Cu (II) is in soluble form. A suitable source is for example cupric sulphate. The treatment with Cu (II) is suitably carried out in an aqueous medium, particularly an aqueous solution, and typically in a buffer, for example an orthophosphate buffer.

The lipid may contain at least one aliphatic carbon-carbon double bond; it may comprise a fatty acid which has at least one such bond.

The lipid may be a lipoprotein. In one class of embodiments the lipoprotein is an HDL. In another class of embodiments the lipoprotein is an LDL. The lipoprotein, e.g. HDL or LDL, may be of mammalian origin, and it is of bovine origin in some embodiments. In other embodiments the lipoprotein is of human origin. Alternative lipoprotein origins are included in the invention. Also to be mentioned as the lipid are other cholesterol-containing lipids, for example VLDL and chylomicrons (whether natural chylomicrons or artificial chylomicron mimics).

The neutralising activity of a lipid may be generated by causing or allowing it to be oxidised, for example by using an oxidising agent, e.g. Cu (II). In this latter case, the speed at which neutralising activity is generated has been observed to be directly proportional to the concentration of Cu (II). In embodiments, the concentration of Cu (II) is at least about 0.01 mM and may be no more that 5 mM. The concentration may be at least 0.03 mM, e.g. about 0.05 mM or more, for example 0.1 mM or more. Often the concentration of Cu (II) is no more than 3 mM; a particular Cu (II) concentration range is from 0.1 to 1 mM; a concentration of about 1 mM is preferred in one embodiment. The Cu (II) concentrations mentioned in this paragraph are in particular applicable to embodiments in which the lipid is in the form of a lipoprotein, e.g. LDL or, in other embodiments, HDL.

The disclosure includes embodiments in which the lipid is a lipoprotein and is treated with Cu (II) at lipoprotein dilutions (lipoprotein in aqueous medium, e.g. buffer) in the range of 1 in 3 up to 1 in 81 and more preferably is in the range of 1 in 5 to 1 in 20 and more preferably still is about 1 in 9. We have observed that a dilution of 1 in 9 generates a high level of neutralising activity within 6 hours. These dilutions refer to dilution with a suitable buffer, e.g. a sodium orthophosphate buffer.

Included are methods in which the lipid is derived from plasma, e.g. citrated plasma.

In some preferred embodiments, the plasma is human plasma. In other preferred embodiments, the plasma is bovine plasma. Alternatively, it may be another mammalian plasma. The oxidised lipid may be obtainable from plasma by gel filtration.

Oxidation of plasma generates an activity that neutralises the activity of the compounds of Formula (IX) below, their salts and other peptide boronates. The principal neutralising activity within plasma, as determined by gel filtration, was found to be within the oxidised lipid fraction. High neutralising activity towards TRI-50b and other peptide boronates has been observed in various commercial preparations of purified lipoprotein.

In principle, the product may comprise any lipid peroxide, for example any lipid comprising an unsaturated, especially polyunsaturated (e.g. di-unsaturated), aliphatic group. Suitably, the product comprises a saponifiable lipid. Preferably, the product comprises a peroxide of a sterol compound, for example a cholesteryl ester peroxide. One class of suitable lipids for forming such peroxides comprises esters of a sterol with an unsaturated fatty acid, preferably a polyunsaturated fatty acid; a preferred sterol is cholesterol. Plasma lipids, e.g. human or bovine plasma lipids, are particularly useful. One example of a cholesteryl ester peroxide is cholesteryl linoleate hydroperoxide. The lipid peroxide may be as a free molecule or a lipoprotein, for example an LDL or an HDL.

Advantageously, the product is relatively stable in plasma. For example a useful candidate compound would comprise an unsaturated fatty acid chain that has the potential for forming a peroxide that is not destroyed by endogenous plasma peroxidases, or at least not rapidly destroyed by plasma peroxidases.

Data in this specification demonstrate that a pure lipoprotein preparation of cholesteryl linoleate hydroperoxide is able to rapidly neutralise TRI 50b; it is estimated that approximately 200 molecules of TRI 50b were neutralised per lipoprotein particle. The reaction is stoichiometric indicating consumption of the hydroperoxide rather than catalysis of neutralisation by it.

According to a yet further aspect of the invention there is provided a pharmaceutical comprising a product described herein. Preferably, the pharmaceutical is for use in therapeutically neutralising an organoboronate and especially a boropeptidyl serine protease inhibitor; it may be for use in treating bleeding resulting from the administration of a boropeptidyl inhibitor of a coagulation serine protease. The disclosed products advantageously provide a product that can therapeutically neutralise the activity of a boropeptidyl serine protease inhibitor and thus terminate or reduce or reverse its therapeutic effect if required.

Included in the disclosure are pharmaceutical formulations which comprise a lyophilisate comprising the active compound. Alternatively, the product (medicament) used to neutralise a boropeptide may comprise an aqueous medium containing a lipoprotein peroxide, for example a parenteral nutrition solution or a fraction or derivative thereof.

The pharmaceutical formulations may comprise a pharmaceutically acceptable diluent, carrier or excipient as well as the active neutralising agent. Thus, there is provided a method of production of a pharmaceutical composition for use in treating an unwanted condition resulting from administration of a boropeptide drug, comprising combining a product of the present invention with a pharmaceutically acceptable diluent, carrier or excipient to form a composition for such use.

The disclosed products may be used for:

the manufacture of a medicament for treating bleeding induced by administration of an organoboronate inhibitor of a coagulation serine protease, e.g. induced by a boropeptidyl inhibitor of thrombin;
the manufacture of a medicament for therapeutically neutralising an organoboronate drug (e.g. having its boronate moiety bonded to an sp³ carbon atom), for example, an inhibitor of a coagulation serine protease, e.g. a boropeptidyl inhibitor of thrombin or proteasome.

Thus, there is provided a method of treating an individual in need of reversal of the activity of a biologically active boronate, for example an organoboronate drug, comprising administering a therapeutically effective amount of a product of the present invention to the individual. The boronate or drug may be a peptidyl boronate or another compound having its boronate moiety bonded to an sp³ carbon atom, for example, it may be a boropeptidyl inhibitor of a coagulation serine protease, e.g. a boropeptidyl inhibitor of thrombin or proteasome

Preferably, the product is administered in an amount such that there is a molar equivalence ratio of approximately 1:1 with the organoboronate in the individual's plasma. A blood level of 0.1 to 1 μM, e.g. 0.25 to 0.75 μM, especially about 0.5 μM, of the product may be sufficient to cause rapid reversal of the activity of that level of a boropeptide or other organoboronate in blood.

Target Compounds

The products of the disclosure are useful for reducing or substantially destroying the clinically significant activity of a biologically active boronate species and particularly of organoboronate drugs. Such reduction of substantial destruction of clinically significant activity of drugs are convenience referred to herein as “neutralisation”. In particular, the disclosed products find application in neutralising aminoboronates or peptidoboronates as described in more detail below.

Typically, the boronate group (—B(OH)₂ or a salt or prodrug form thereof) of the target compound is bonded to an aliphatic carbon atom and normally to an sp³ carbon atom. The target compound may for example be any boronic acid drug mentioned under the heading “BACKGROUND” or in any document referred to under that heading, e.g. it may be TRI 50c or Velcade®. It may be a boronic acid described in WO 01/02424. Particular boronic acid drugs are peptide boronic acids, including those having a C-terminal residue which is of an α-aminoboronic acid having an alkyl or alkoxyalkyl side chain. An exemplary C-terminal residue is of Boro-3-methoxypropylglycine, as for example in the case that the drug comprises a boropeptide which includes the sequence Pro-Mpg-B(OH)₂, for example as part of the larger sequence Phe-Pro-Mpg-B(OH)₂, whether administered as the free acid, a salt or a prodrug. In this paragraph, reference to a boronic acid described in the prior art includes reference to the free acids and salts of boronate esters described in the prior art. It may be any other boronic acid drug.

The target compounds for neutralisation by the products of the invention, therefore, are organoboronates and especially boropeptides. Organoboronates (e.g. peptide boronates) can exist in different forms, such as acids, esters and tautomers, for example, and the target compounds of the present invention includes all variant forms of the compounds. Whilst pharmaceutically useful boronic acids may be administered as the free acid, they may also be administered in other forms, e.g. as anhydrides, salts, anhydride salts, esters or other prodrugs.

Thus, many organoboronates include basic groups and may therefore be administered in the form of acid addition salts. Exemplary acids include HBr, HCl and HSO₂CH₃. Alternatively the organoboronates may be administered in the form of base addition salts thereof as described in WO 2004/022072, U.S. Ser. No. 10/659,178 and EP-A-1396270; WO 2004/022071, U.S. Ser. No. 10/659,179 and EP-A-1396269; and also in WO 2004/022070, U.S. Ser. No. 10/658,971 and EP-A-1400245. Salts of alkali metal and alkaline earth metals, e.g. sodium and calcium, are representative of base addition salts as well as salts of organic bases, e.g. N-methyl-D-glucamine. The target organoboronate drugs may be administered as esters, notably esters of diols; exemplary diols in particular are sugars, for example mannitol, as described in WO 02/059131 and U.S. Pat. No. 6,699,835.

Further, there is a debate in the literature as to whether boronates in aqueous solution form the ‘trigonal’ B(OH)₂ or ‘tetrahedral’ B(OH)⁻₃ boron species, and representations of trigonal B(OH)₂ include reference to tetrahedral as well as trigonal boron species.

The target compounds of the invention therefore include all variant forms of the substances concerned, for example any tautomer or any pharmaceutically acceptable salt, ester, acid or other variant of the substances and their tautomers as well as substances which, upon administration, are capable of providing directly or indirectly such substances or providing a species which is capable of existing in equilibrium with such a substance.

In certain embodiments the organoboronic acid is hydrophobic.

Included herein are embodiments in which the organoboronic acid comprises an aminoboronic acid linked through a peptide linkage to an organic moiety, and often a moiety comprising an amino acid (natural or unnatural) or a peptide, which organic moiety may be hydrophobic. The organic moiety can comprise an amino acid whose C-terminal carboxy group forms part of said peptide linkage. The target compound may therefore be of formula (XIII):

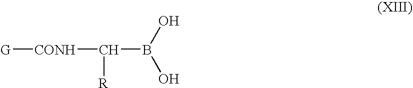

In formula (XIII), G is an organic moiety, for example comprising together with —CO— a residue of an optionally N-terminally substituted amino acid or peptide (e.g. dipeptide), a suitable N-terminal substituent being for example an X group as described below. R is a side chain of an amino acid (whether natural or unnatural). G and R may be hydrophobic. R may be an R¹ group as described below. Peptide linkages in formula (XIII) compounds are optionally and independently N-substituted, for example by a C₁-C₁₃ hydrocarbyl optionally containing in-chain oxygen or sulfur and optionally substituted by a substituent selected from halo, hydroxy and trifluoromethyl (an example of such an N-substituent is 1 C to 6 C alkyl).

One specific class of target compounds comprises those wherein the organoboronic acid comprises a boropeptide or boropeptidomimetic. For example, in a sub-class of these organoboronates the organoboronic acid is of the formula (VIII): 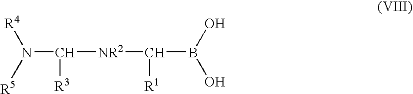
where:
R¹ is H or a non-charged side group;
R² is H or C₁-C₁₃ hydrocarbyl optionally containing in-chain oxygen or sulfur and optionally substituted by a substituent selected from halo, hydroxy and trifluoromethyl;
or R¹ and R² together form a C₁-C₁₃ moiety which in combination with N—CH forms a 4-6 membered ring and which is selected from alkylene (whether branched or linear) and alkylene containing an in-chain sulfur or linked to N—CH through a sulfur;
   R³ is the same as or different from R¹ provided that no more than one of R¹ and R² is H;
   R⁴ is H or a C₁-C₁₃ hydrocarbyl group optionally containing in-chain oxygen or sulfur and optionally substituted by a substituent selected from halo, hydroxy and trifluoromethyl;
or R³ and R⁴ together form a C₁-C₁₃ moiety which in combination with N—CH forms a 4-6 membered ring and which is selected from alkylene (whether branched or linear) and alkylene containing an in-chain sulfur or linked to N—CH through a sulfur; and
   R⁵ is X-E- wherein E is nothing or a hydrophobic moiety selected from the group consisting of amino acids (natural or unnatural) and peptides of two or more amino acids (natural or unnatural) of which more than half are hydrophobic, in which peptides the nitrogen(s) of the peptide linkage(s) may be substituted by a C₁-C₁₃ hydrocarbyl optionally containing in-chain oxygen or sulfur and optionally substituted by a substituent selected from halo, hydroxy and trifluoromethyl (an example of such an N-substituent is 1 C to 6 C alkyl), and X is H or an amino-protecting group.

Said C₁-C₁₃ hydrocarbyl optionally containing in-chain oxygen or sulfur may be selected from alkyl; alkyl substituted by cycloalkyl, aryl or heterocyclyl; cycloalkyl; aryl; and/or heterocyclyl. Heterocyclyl may be heteroaryl.

R¹ may be non polar. In some embodiments, R¹ contains up to 20 carbon atoms. R¹ may have affinity for the S1 subsite of a protease.

In a preferred class of boronic acids, which are anti-thrombotic and include TRI 50c, the acid has a neutral moiety capable of binding to the thrombin S1 subsite linked to a hydrophobic moiety capable of binding to the thrombin S2 and S3 subsites. The acid may for example be of formula (III): 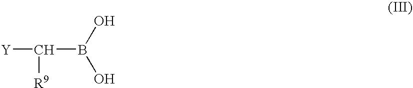
wherein
Y comprises a moiety which, together with the fragment —CH(R⁹)—B(OH)₂, has affinity for the substrate binding site of thrombin; and
R⁹ is a straight chain alkyl group interrupted by one or more ether linkages (e.g. 1 or 2) and in which the total number of oxygen and carbon atoms is 3, 4, 5 or 6 (e.g. 5) or R⁹ is —(CH₂)ₘ—W where m is 2, 3, 4 or 5 (e.g. 4) and W is —OH or halogen (F, Cl, Br or I). As examples of straight chain alkyl interrupted by one or more ether linkages (—O—) may be mentioned alkoxyalkyl (one interruption) and alkoxyalkoxyalkyl (two interruptions). R⁹ is an alkoxyalkyl group in one subset of compounds, e.g. alkoxyalkyl containing 4 carbon atoms.

The neutral aminoboronic acid residue capable of binding to the thrombin S1 subsite may be linked through a peptide linkage to a hydrophobic moiety capable of binding to the thrombin S2 and S3 subsites. As a class of such compounds may be mentioned acids of formula (IX): 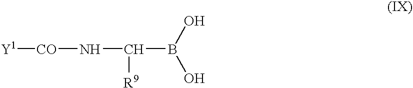
wherein
Y¹ comprises a hydrophobic moiety which, together with the aminoboronic acid residue —NHCH(R⁹)—B(OH)₂, has affinity for the substrate binding site of thrombin; and
R⁹ is as defined above.

Typically, YCO— comprises an amino acid residue (whether natural or unnatural) which binds to the S2 subsite of thrombin, the amino acid residue being N-terminally linked to a moiety which binds the S3 subsite of thrombin. Peptide linkages in the acid of formula (IX) may be substituted or unsubstituted; in one class of embodiments they are unsubstituted.

In one class of Formula (IX) acids, YCO— is an optionally N-terminally protected dipeptide residue which binds to the S3 and S2 binding sites of thrombin and the peptide linkages in the acid are optionally and independently N-substituted by a C₁-C₁₃ hydrocarbyl group optionally containing in-chain and/or in-ring nitrogen, oxygen or sulfur and optionally substituted by a substituent selected from halo, hydroxy and trifluoromethyl. The N-terminal protecting group, when present, may be a group X as defined above (other than hydrogen). Normally, the acid contains no N-substituted peptide linkages; where there is an N-substituted peptide linkage, the substituent is often 1 C to 6 C hydrocarbyl, e.g. saturated hydrocarbyl; the N-substituent comprises a ring in some embodiments, e.g. cycloalkyl, and may be cyclopentyl, for example. One class of acids has an N-terminal protecting group (e.g. an X group) and unsubstituted peptide linkages.

Where YCO— is a dipeptide residue (whether or not N-terminally protected), the S3-binding amino acid residue may be of R configuration and/or the S2-binding residue may of S configuration. The fragment —NHCH(R⁹)—B(OH) may of R configuration. The disclosure is not restricted to chiral centres of these conformations, however.

In one class of compounds, the side chain of P3 (S3-binding) amino acid and/or the P2 (S2-binding) amino acid is a moiety other than hydrogen selected from a group of formula A or B:
—(CO)ₐ—(CH₂)_{b}-D_{c}-(CH₂)_{d}-E (A)
—(CO)ₐ—(CH₂)_{b}-D_{c}-Cₑ(E¹)(E²)(E³) (B)
wherein
a is 0 or 1;
e is 1;
b and d are independently 0 or an integer such that (b+d) is from 0 to 4 or, as the case may be, (b+e) is from 1 to 4;
c is 0 or 1;
D is O or S;
E is H, C₁-C₆ alkyl, or a saturated or unsaturated cyclic group which normally contains up to 14 members and particularly is a 5-6 membered ring (e.g. phenyl) or an 8-14 membered fused ring system (e.g. naphthyl), which alkyl or cyclic group is optionally substituted by up to 3 groups (e.g. 1 group) independently selected from C₁-C₆ trialkylsilyl, —CN, —R¹³, —R¹²OR¹³, —R¹²COR¹³, —R¹²CO₂R¹³ and —R¹²O₂CR¹³, wherein R¹² is —(CH₂)_{f}— and R¹³ is —(CH₂)_{g}H or by a moiety whose non-hydrogen atoms consist of carbon atoms and in-ring heteroatoms and number from 5 to 14 and which contains a ring system (e.g. an aryl group) and optionally an alkyl and/or alkylene group, wherein f and g are each independently from 0 to 10, g particularly being at least 1 (although —OH may also be mentioned as a substituent), provided that (f+g) does not exceed 10, more particularly does not exceed 6 and most particularly is 1, 2, 3 or 4, and provided that there is only a single substituent if the substituent is a said moiety containing a ring system, or E is C₁-C₆ trialkylsilyl; and E¹, E² and E³ are each independently selected from —R¹⁵ and -J-R¹⁵, where J is a 5-6 membered ring and R¹⁵ is selected from C₁-C₆ trialkylsilyl, —CN, —R¹³, —R¹²OR¹³, —R¹²COR¹³, —R¹²CO₂R¹³, —R¹²O₂CR¹³, and one or two halogens (e.g. in the latter case to form a -J-R¹⁵ moiety which is dichlorophenyl), where R¹² and R¹³ are, respectively, an R¹² moiety and an R¹³ moiety as defined above (in some acids where E¹, E² and E³ contain an R¹³ group, g is 0 or 1);
in which moiety of Formula (A) or (B) any ring is carbocyclic or aromatic, or both, and any one or more hydrogen atoms bonded to a carbon atom is optionally replaced by halogen, especially F.

In certain examples, a is 0. If a is 1, c may be 0. In particular examples, (a+b+c+d) and (a+b+c+e) are no more than 4 and are more especially 1, 2 or 3. (a+b+c+d) may be 0.

Exemplary groups for E, E¹, E² and E³ include aromatic rings such as phenyl, naphthyl, pyridyl, quinolinyl and furanyl, for example; non-aromatic unsaturated rings, for example cyclohexenyl; saturated rings such as cyclohexyl, for example. E may be a fused ring system containing both aromatic and non-aromatic rings, for example fluorenyl. One class of E, E¹, E² and E³ groups are aromatic (including heteroaromatic) rings, especially 6-membered aromatic rings. In some compounds, E¹ is H whilst E² and E³ are not H; in those compounds, examples of E² and E³ groups are phenyl (substituted or unsubstituted) and C₁-C₄ alkyl, e.g. methyl.

In one class of boronic acids, E contains a substituent which is C₁-C₆ alkyl, (C₁-C₅ alkyl)carbonyl, carboxy C₁-C₅ alkyl, aryl (including heteroaryl), especially 5-membered or preferably 6-membered aryl (e.g. phenyl or pyridyl), or arylalkyl (e.g. arylmethyl or arylethyl where aryl may be heterocyclic and is preferably 6-membered).

In another class of boronic acids, E contains a substituent which is OR¹³, wherein R¹³ can be a 6-membered ring, which may be aromatic (e.g. phenyl) or is alkyl (e.g. methyl or ethyl) substituted by such a 6-membered ring.

A class of moieties of formula A or B are those in which E is a 6-membered aromatic ring optionally substituted, particularly at the 2-position or 4-position, by —R¹³ or —OR¹³.

Also to be mentioned are boronic aid thrombin inhibitors in which the P3 and/or P2 side chain comprises a cyclic group in which 1 or 2 hydrogens have been replaced by halogen, e.g. F or Cl. Further to be mentioned is a class of organoboronic acid in which the side chains of formula (A) or (B) are of the following formulae (C), (D) or (E): 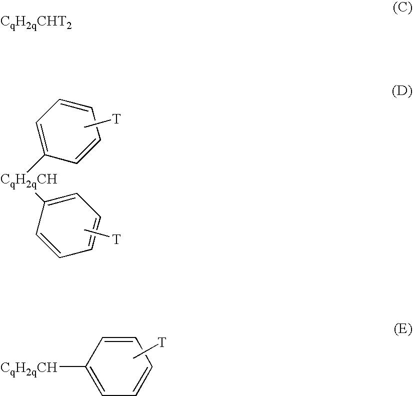
wherein q is from 0 to 5, e.g. is 0, 1 or 2, and each T is independently hydrogen, one or two halogens (e.g. F or Cl), —SiMe₃, —CN, —R¹³, —OR¹³, —COR¹³, —CO₂R¹³ or —O₂CR¹³. In some embodiments of structures (D) and (E), T is at the 4-position of the phenyl group(s) and is —R¹³, —OR¹³, —COR¹³, —CO₂R¹³ or —O₂CR¹³, and R¹³ is C₁-C₁₀ alkyl and more particularly C₁-C₆ alkyl. In one sub-class, T is —R¹³ or —OR¹³, for example in which f and g are each independently 0, 1, 2 or 3; in some side chains groups of this sub-class, T is —R¹²OR¹³ and R¹³ is H.

In one class of the moieties, the side chain is of formula (C) and each T is independently R¹³ or OR¹³ and R¹³ is C₁-C₄ alkyl. In some of these compounds, R¹³ is branched alkyl and in others it is straight chain. In some moieties, the number of carbon atoms is from 1 to 4.

In many dipeptide fragments YCO— (which dipeptides may be N-terminally protected or not), the P3 amino acid has a side chain of formula (A) or (B) as described above and the P2 residue is of an imino acid.

The target compounds may therefore be organoboronic acids which are thrombin inhibitors, particularly selective thrombin inhibitors, having a neutral P1 (S1-binding) moiety. For more information about moieties which bind to the S3, S2 and S1 sites of thrombin, see for example Tapparelli C et al, Trends Pharmacol. Sci. 14: 366-376, 1993; Sanderson P et al, Current Medicinal Chemistry, 5: 289-304, 1998; Rewinkel J et al, Current Pharmaceutical Design, 5:1043-1075, 1999; and Coburn C Exp. Opin. Ther. Patents 11(5): 721-738, 2001. The thrombin inhibitory compounds are not limited to those having S3, S2 and S1 affinity groups described in the publications listed in the preceding sentence.

The boronic acids may have a Ki for thrombin of about 100 nM or less, e.g. about 20 nM or less.

A subset of the Formula (IX) acids comprises the acids of Formula (X):

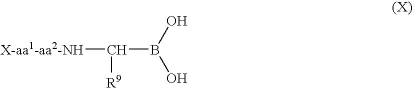

X is a moiety bonded to the N-terminal amino group and may be H to form NH₂. The identity of X is not critical but may be a particular X moiety described above. In one example there may be mentioned benzyloxycarbonyl.

In certain examples X is R⁶—(CH₂)ₚ—C(O)—, R⁶—(CH₂)ₚ—S(O)₂—, R⁶—(CH₂)ₚ—NH—C(O)— or R⁶—(CH₂)ₚ—O—C(O)— wherein p is 0, 1, 2, 3, 4, 5 or 6 (of which 0 and 1 are preferred) and R⁶ is H or a 5 to 13-membered cyclic group optionally substituted by 1, 2 or 3 substituents selected from halogen, amino, nitro, hydroxy, a C₅-C₆ cyclic group, C₁-C₄ alkyl and C₁-C₄ alkyl containing, and/or linked to the 5 to 13-membered cyclic group through, an in-chain O, the aforesaid alkyl groups optionally being substituted by a substituent selected from halogen, amino, nitro, hydroxy and a C₅-C₆ cyclic group. More particularly X is R⁶—(CH₂)ₚ—C(O)— or R⁶—(CH₂)ₚ—O—C(O)— and p is 0 or 1. Said 5 to 13-membered cyclic group is often aromatic or heteroaromatic, for example is a 6-membered aromatic or heteroaromatic group. In many cases, the group is not substituted.

Exemplary X groups are (2-pyrazine)carbonyl, (2-pyrazine)sulfonyl and particularly benzyloxycarbonyl.

aa¹ is an amino acid residue having a hydrocarbyl side chain containing no more than 20 carbon atoms (e.g. up to 15 and optionally up to 13 C atoms) and comprising at least one cyclic group having up to 13 carbon atoms. In certain examples, the cyclic group(s) of aa¹ have/has 5 or 6 ring members. For instance, the cyclic group(s) of aa¹ may be aryl groups, particularly phenyl. Typically, there are one or two cyclic groups in the aa¹ side chain. Certain side chains comprise, or consist of, methyl substituted by one or two 5- or 6-membered rings.

More particularly, aa¹ is Phe, Dpa or a wholly or partially hydrogenated analogue thereof. The wholly hydrogenated analogues are Cha and Dcha.

aa² is an imino acid residue having from 4 to 6 ring members. Alternatively, aa² is Gly N-substituted by a C₃-C₁₃ hydrocarbyl group, e.g. a C₃-C₈ hydrocarbyl group comprising a C₃-C₆ hydrocarbyl ring; the hydrocarbyl group may be saturated, for example exemplary N-substituents are cyclopropyl, cyclobutyl, cyclopentyl and cyclohexyl. As a hydrocarbyl group containing one or more unsaturated bonds may be mentioned phenyl and methyl or ethyl substituted by phenyl, e.g. 2-phenylethyl, as well as, -dialkylphenylethyl.

An exemplary class of products comprises those in which aa² is a residue of an imino acid of formula (XI) 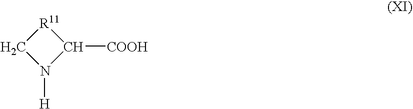
where R¹¹ is —CH₂—, CH₂—CH₂—, —S—CH₂— or —CH₂—CH₂—CH₂—, which group when the ring is 5 or 6-membered is optionally substituted at one or more —CH₂— groups by from 1 to 3 C₁-C₃ alkyl groups, for example to form the R¹¹ group —S—C(CH₃)₂—. Of these imino acids, azetidine-2-carboxylic acid, especially (s)-azetidine-2-carboxylic acid, and more particularly proline are illustrative.

It will be appreciated from the above that a particular class of organoboronates consists of those in which aa¹-aa² is Phe-Pro. In another preferred class, aa¹-aa² is Dpa-Pro. In other products, aa¹-aa² is Cha-Pro or Dcha-Pro. Of course, also included are corresponding product classes in which Pro is replaced by (s)-azetidine-2-carboxylic acid.

R⁹ is as defined previously and may be a moiety R¹ of the formula —(CH₂)ₛ-Z. Integer s is 2, 3 or 4 and W is —OH, -OMe, -OEt or halogen (F, Cl, I or, preferably, Br). Particularly illustrative Z groups are -OMe and -OEt, especially -OMe. In certain examples s is 3 for all Z groups and, indeed, for all compounds of the disclosure. Particular R¹ groups are 2-bromoethyl, 2-chloroethyl, 2-methoxyethyl, 4-bromobutyl, 4-chlorobutyl, 4-methoxybutyl and, especially, 3-bromopropyl, 3-chloropropyl and 3-methoxypropyl. Most preferably, R¹ is 3-methoxypropyl. 2-Ethoxyethyl is another preferred R¹ group.

A specific class of target compounds comprises boropeptides having the amino acid sequence Phe-Pro-BoroMpg, particularly (R)-Phe-(S)-Pro-(R)-BoroMpg. Thus, there may be mentioned acids of the formula X-Phe-Pro-Mpg-B(OH)₂, especially Cbz-Phe-Pro-Mpg-B(OH)₂; also included are analogues of these compounds in which Mpg is replaced by a residue with another of the R¹ groups and/or Phe is replaced by Dpa or another aa¹ residue.

The aa¹ moiety is preferably of R configuration. The aa² moiety is preferably of (S)-configuration. Particularly preferred target compounds of formula (III) have aa¹ of (R)-configuration and aa² of (S)-configuration. The chiral centre —NH—CH(R¹)—B— is preferably of (R)-configuration. It is considered that commercial formulations will have the chiral centres in (R,S,R) arrangement, as for example in the case of Cbz-Phe-Pro-BoroMpg-OH: 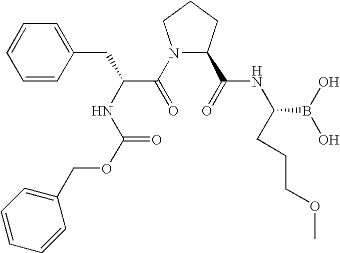 (R,S,R)-TRI 50c Cbz-(R)-Phe-(S)-Pro-(R)-Mpg-B(OH)₂

The target boronic acids may of course be administered in any form which results in release of the free acid or a corresponding boronate anion, e.g. as anhydrides, salts, anhydride salts or prodrugs thereof. All the boronic acids described herein may therefore be administered in the form of prodrugs, or as the reaction product (salt) of combining the boronic acid or a prodrug thereof with a pharmaceutically acceptable acid or base, and the disclosed reversal agents may be used following administration of a boronic acid drug in free form or in salt or prodrug form.

As suitable prodrugs may be mentioned esters, e.g. with a residue of an alkanol, e.g. a C₁-C₄ alkanol such as methanol or ethanol, for example. It may be an ester of a diol.

The identity of the diol is not critical. As suitable diols may be mentioned aliphatic and aromatic compounds having hydroxy groups that are substituted on adjacent carbon atoms or on carbon atoms substituted by another carbon. That is to say, suitable diols include compounds having at least two hydroxy groups separated by at least two connecting carbon atoms in a chain or ring. One class of diols comprises hydrocarbons substituted by exactly two hydroxy groups. One such diol is pinacol and another is pinanediol and a third is diethanolamine; there may also be mentioned neopentylglycol, 1,2-ethanediol, 1,2-propanediol, 1,3-propanediol, 2,3-butanediol, 1,2-diisopropylethanediol, 5,6-decanediol and 1,2-dicyclohexylethanediol.

The prodrug may be a sugar derivative as described in WO 02/059131 (see above). Thus, the boronate group may be esterified with a sugar such as a monosaccharide or disaccharide, for example. The sugar may be a reduced sugar, e.g. mannitol or sorbitol; it may be any individual sugar or class of sugars taught in WO 02/059131. The boronic acid, sugar (or other diol) and water may be combined and then lyophilised, for example as taught in WO 02/059131.

As salts, there may be mentioned:

1. Alkali metal salts;

2. Divalent, e.g. alkaline earth metal, salts;

3. Group III metals;

4. Salts of strongly basic organic nitrogen-containing compounds, including:

4A. Salts of guanidines and their analogues;
4B. Salts of strongly basic amine, examples of which include (i) aminosugars and (ii) other amines.

Of the above salts, particularly illustrative are alkali metals, especially Na and Li, and alkaline earth metals, especially magnesium and calcium. Also illustrative are aminosugars. The term “salt” herein does not imply any particular structure at the molecular level but refers merely to a product formed by contacting together an acid and a base.

Specific salts are of the acid boronate though in practice the acid salts may contain a very small proportion of the doubly deprotonated boronate. The term “acid boronate” refers to trigonal —B(OH)₂ groups in which one of the B—OH groups is deprotonated as well as to corresponding tetrahedral groups in equilibrium therewith. Acid boronates have a stoichiometry consistent with single deprotonation.

Suitable organic bases include those with a pKb of 7 or more, e.g. 7.5 or more, for example in the region of 8 or more. Bases which are less lipophilic [e.g. have at least one polar functional group (e.g. 1, 2 or 3 such groups) for example hydroxy] are favoured; thus aminosugars are one favoured class of base, for example N-methyl-D-glucamine. Other organic bases to be mentioned are arginine and lysine.

Use of the Products of the Disclosure

The products of the disclosure may be used as reversal agents (also known as antidotes) for organoboronate drugs, e.g. one described herein under the heading “Target Compounds”. They may therefore be administered when an organoboronate drug has produced unwanted side effects, e.g. bleeding after administration of an anticoagulant. The products of the disclosure may also be used in any circumstances where an organoboronate compound has been ingested or absorbed and is causing toxicity.

The disclosure includes a method of preparing to supply a first pharmaceutical composition for the treatment of unwanted coagulation (e.g. thrombosis) by prophylaxis or therapy and, if required, a second pharmaceutical composition to inhibit the action of the first composition, comprising stocking a pharmaceutical composition comprising a target compound as hereinbefore described and stocking a pharmaceutical formulation or medicament comprising a product of the present disclosure.

According to a yet further aspect of the disclosure there is provided a method of providing a medicament pair, the pair comprising a first medicament for the treatment of unwanted coagulation (e.g. thrombosis) by prophylaxis or therapy and a second medicament to inhibit the action of the first medicament in the event of undue bleeding, wherein the first medicament comprises a target compound as hereinbefore described and the second medicament comprises a pharmaceutical composition or medicament according to the present disclosure.

According to a yet further aspect of the disclosure there is provided a method for treating unwanted coagulation (e.g. thrombosis) by prophylaxis or therapy, or inhibiting thrombosis in the treatment of disease by prophylaxis or therapy, using an anticoagulant which results in inappropriate bleeding and then inhibiting the action of said anticoagulant, wherein a therapeutically effective amount of an anticoagulant composition comprising a target compound as hereinbefore described is administered to a patient in need thereof, or to an extracorporeal blood circuit of a patient, to treat coagulation or inhibit coagulation in the treatment of disease (including in treatment by surgery), and, after the inappropriate bleeding, a therapeutically effective amount of a product the disclosure is administered to a patient to inhibit the anticoagulant.

It may be mentioned by way of non-limiting example that the described active products and pharmaceutical formulations may administered orally. More typically, they may be administered intravenously.

According to a yet further aspect of the disclosure there is provided use, for the manufacture of a medicament pair comprising a first medicament for treating unwanted coagulation (e.g. thrombosis) by prophylaxis or therapy and a second medicament for, if required, stopping or reducing the anticoagulant treatment, of a target compound as herein before described, for the manufacture of the first medicament and a product of the present disclosure for the manufacture of the second medicament.

According to a yet further aspect of the disclosure there is provided use, for the manufacture of a medicament pair comprising a first medicament for treating unwanted coagulation (e.g. thrombosis) by prophylaxis or therapy and a second medicament for, if required, stopping or reducing undue or inappropriate bleeding caused by the first medicament, of a target compound as herein before described for the manufacture of the first medicament and a product according to the present disclosure for the manufacture of the second medicament.

According to a yet further aspect of the disclosure there is provided a method of preparing for the administration to a patient or an extracorporeal blood circuit of a first pharmaceutical composition for the treatment of unwanted coagulation (e.g. thrombosis) by prophylaxis or therapy and, if required, a second pharmaceutical composition for reacting with the active agent of the first composition to inactivate molecules thereof, comprising supplying a pharmaceutical composition comprising a target compound, as hereinbefore described, and supplying a pharmaceutical composition or a medicament according to the present disclosure.

Particularly, either or both of the first and second pharmaceutical compositions are administered orally and/or intravenously.

Preferred target compounds are thrombin inhibitors. They are therefore useful for inhibiting thrombin. There are therefore provided compounds which have potential for controlling haemostasis and especially for inhibiting coagulation, for example in the treatment or prevention of secondary events after myocardial infarction. The medical use of the compounds may be prophylactic (including to treat thrombosis as well as to prevent occurrence of thrombosis) as well as therapeutic (including to prevent re-occurrence of thrombosis or secondary thrombotic events).

The anticoagulant target compounds may be employed when an anti-thrombogenic agent is needed. Further, it has been found that anti-thrombotic target compounds, including those of boronic acids of Formula (IX), are beneficial in that the class is useful for treating arterial thrombosis by therapy or prophylaxis. The target compounds are thus indicated in the treatment or prophylaxis of thrombosis and hypercoagulability in blood and tissues of animals including man. The term “thrombosis” includes inter alia atrophic thrombosis, arterial thrombosis, cardiac thrombosis, coronary thrombosis, creeping thrombosis, infective thrombosis, mesenteric thrombosis, placental thrombosis, propagating thrombosis, traumatic thrombosis and venous thrombosis.

It is known that hypercoagulability may lead to thromboembolic diseases.

Particular uses which may be mentioned for boronic acid inhibitors of coagulation serine proteases, e.g. thrombin inhibitors, include the therapeutic and/or prophylactic treatment of venous thrombosis and pulmonary embolism. Preferred indications envisaged for the described thrombin inhibitory boronic acids (notably the salts of TRI 50c) include:
Prevention of venous thromboembolic events (e.g. deep vein thrombosis and/or pulmonary embolism). Examples include patients undergoing orthopaedic surgery such as total hip replacement, total knee replacement, major hip or knee surgery; patients undergoing general surgery at high risk for thrombosis, such as abdominal or pelvic surgery for cancer; and in patients bedridden for more than 3 days and with acute cardiac failure, acute respiratory failure, infection.
Prevention of thrombosis in the haemodialysis circuit in patients, in patients with end stage renal disease.
Prevention of cardiovascular events (death, myocardial infarction, etc) in patients with end stage renal disease, whether or not requiring haemodialysis sessions.
Prevention of venous thrombo-embolic events in patients receiving chemotherapy through an indwelling catheter.
Prevention of thromboembolic events in patients undergoing lower limb arterial reconstructive procedures (bypass, endarteriectomy, transluminal angioplasty, etc).
Treatment of venous thromboembolic events.
Prevention of cardiovascular events in acute coronary syndromes (e.g. unstable angina, non Q wave myocardial ischaemia/infarction), in combination with another cardiovascular agent, for example aspirin (acetylsalicylic acid; aspirin is a registered trade mark in Germany), thrombolytics (see below for examples), antiplatelet agents (see below for examples).
Treatment of patients with acute myocardial infarction in combination with acetylsalicylic acid, thrombolytics (see below for examples).
Prevention of unwanted bleeding during coronary artery bypass graft procedures.
   Administration and Pharmaceutical Formulations

The disclosed products may be administered to a host, for example, when an organoboronate drug is resulting in undesired effects which it is wished to stop or reduce. In the case of larger animals, such as humans, the products may be administered alone or in combination with pharmaceutically acceptable diluents, excipients or carriers. The term “pharmaceutically acceptable” includes acceptability for both human and veterinary purposes, of which acceptability for human pharmaceutical use is preferred.

The products of the disclosure may be combined and/or co-administered with another medicament. For example, they may be combined and/or co-administered with a procoagulant when the target boronic acid drug is an anticoagulant.

Actual dosage levels of active ingredients in the pharmaceutical compositions of this disclosure may be varied so as to obtain an amount of the active product(s) that is effective to achieve the desired therapeutic response for a particular patient, composition, and mode of administration (referred to herein as a “therapeutically effective amount”). The selected dosage level will depend upon the activity of the particular product, the severity of the condition being treated and the condition and prior medical history of the patient being treated. However, it is within the skill of the art to start doses of the product at levels lower than required for to achieve the desired therapeutic effect and to gradually increase the dosage until the desired effect is achieved.

According to a further aspect there is provided a parenteral formulation including a product as described herein. The formulation may consist of the product alone or it may contain additional components, in particular the product may be in combination with a pharmaceutically acceptable diluent, excipient or carrier, for example a tonicity agent for the purpose of making the formulation substantially isotonic with the body of the subject to receive the formulation, e.g. with human plasma. The formulation may be in ready-to-use form or in a form requiring reconstitution prior to administration. More particularly, the parenteral formulation may be an intravenous formulation.

A particular embodiment resides in intravenous formulations, whether in liquid ready-to-use form or in solid form for reconstitution, or otherwise, comprising an oxidised lipoprotein, particularly oxidised HDL or LDL. The HDL or LDL may have been prepared by treatment with copper (II). The invention comprises intravenous formulations containing a lipoprotein peroxide, e.g. a hydroperoxide.

Parenteral preparations can be administered by one or more routes, such as intravenous, subcutaneous, intradermal and infusion; a particular example is intravenous. A formulation disclosed herein may be administered using a syringe, injector, plunger for solid formulations, pump, or any other device recognized in the art for parenteral administration.

Liquid dosage forms for parenteral administration may include solutions, suspensions, liposome formulations, or emulsions in oily or aqueous vehicles. In addition to the active products, the liquid dosage forms may contain other compounds. Tonicity agents (for the purpose of making the formulations substantially isotonic with the subject's body, e.g. with human plasma) such as, for instance, sodium chloride, sodium sulfate, dextrose, mannitol and/or glycerol may be optionally added to the parenteral formulation. A pharmaceutically acceptable buffer may be added to control pH. Thickening or viscosity agents, for instance well known cellulose derivatives (e.g. methylcellulose, carboxymethylcellulose, hydroxyethylcellulose and hydroxypropylmethyl-cellulose), gelatin and/or acacia, may optionally be added to the parenteral formulation.

Solid dosage forms for parenteral administration may encompass solid and semi-solid forms and may include pellets, powders, granules, patches, and gels. In such solid dosage forms, the active product is typically mixed with at least one inert, pharmaceutically acceptable excipient or carrier.

The disclosed products may be presented as solids in finely divided solid form, for example they may be milled or micronised.

The formulations may also include antioxidants and/or preservatives. As antioxidants may be mentioned thiol derivatives (e.g. thioglycerol, cysteine, acetylcysteine, cystine, dithioerythreitol, dithiothreitol, glutathione), tocopherols, butylated hydroxyanisole, butylated hydroxytoluene, sulfurous acid salts (e.g. sodium sulfate, sodium bisulfite, acetone sodium bisulfite, sodium metabisulfite, sodium sulfite, sodium formaldehyde sulfoxylate, sodium thiosulfate) and nordihydroguaiareticacid. Suitable preservatives may for instance be phenol, chlorobutanol, benzylalcohol, methyl paraben, propyl paraben, benzalkonium chloride and cetylpyridinium chloride.

The parenteral formulations may be prepared as large volume parenterals (LVPs), e.g. larger than 100 ml, more particularly about 250 ml, of a liquid formulation of the active product. Examples of LVPs are infusion bags. The parenteral formulations may alternatively be prepared as small volume parenterals (SVPs), e.g. about 100 ml or less of a liquid formulation of the active product. Examples of SVPs are vials with solution, vials for reconstitution, prefilled syringes for injection and dual chamber syringe devices.

The formulations of the disclosure include those in which the active product is HDL hydroperoxide or LDL hydroperoxide. The products mentioned in this paragraph, or their salts or prodrugs, may be administered as solutions or suspensions in water, typically containing one or more additives, for example isotonicity agent(s) and/or antioxidant(s). A way to store the products is in solid form, for example as dry powder, and to make them up into solutions for administration prior to administration. Alternatively, the products may be stored as liquid formulations ready for use.

One class of formulations disclosed herein is intravenous formulations. For intravenously administered formulations, the active product or products can be present at varying concentrations, with a carrier acceptable for parenteral preparations making up the remainder.

Particularly, the carrier is water, particularly pyrogen free water, or is aqueous based. Particularly, the carrier for such parenteral preparations is an aqueous solution comprising a tonicity agent, for example a sodium chloride solution.

By “aqueous based” is meant that formulation comprises a solvent which consists of water or of water and water-miscible organic solvent or solvents; as well as containing a product of disclosure in dissolved form, the solvent may have dissolved therein one or more other substances, for example an antioxidant and/or an isotonicity agent. As organic cosolvents may be mentioned those water-miscible solvents commonly used in the art, for example propyleneglycol, polyethyleneglycol 300, polyethyleneglycol 400 and ethanol. Preferably, organic co-solvents are only used in cases where the active agent is not sufficiently soluble in water for a therapeutically effective amount to be provided in a single dosage form. As previously indicated, the disclosure includes formulations of alkali metal salts of the disclosed acids having a solvent which consists of water.

The solubility of the active product in the present formulations may be such that the turbidity of the formulation is lower than 50 NTU, e.g. lower than 20 NTU such as lower than 10 NTU.

It is desirable that parenteral formulations are administered at or near physiological pH. It is believed that administration in a formulation at a high pH (i.e., greater than 8) or at a low pH (i.e., less than 5) is undesirable. In particular, it is contemplated that the formulations would be administered at a pH of between 6.0 and 7.0 such as a pH of 6.5.

The parenteral formulation may be purged of air when being packaged. The parenteral formulation may be packaged in a sterile container, e.g. vial, as a solution, suspension, gel, emulsion, solid or a powder. Such formulations may be stored either in ready-to-use form or in a form requiring reconstitution prior to administration.

Parenteral formulations according to the disclosure may be packaged in containers. Containers may be chosen which are made of material which is non-reactive or substantially non-reactive with the parenteral formulation. Glass containers or plastics containers, e.g. plastics infusion bags, may be used. A concern of container systems is the protection they afford a solution against UV degradation. If desired, amber glass employing iron oxide or an opaque cover fitted over the container may afford the appropriate UV protection.

Plastics containers such as plastics infusion bags are advantageous in that they are relatively light weight and non-breakable and thus more easily stored. This is particularly the case for Large Volume parenterals.

The intravenous preparations may be prepared by combining the active product or products with the carrier. After the formulation is mixed, it may be sterilized, for example using known methods. Once the formulation has been sterilized, it is ready to be administered or packaged, particularly in dark packaging (e.g. bottles or plastics packaging), for storage. It is envisaged, however, that the disclosed products might not be stored in liquid medium but as dry solids, particularly a finely divided form such as, for example, a lyophilisate, in order to prolong shelf life; this would of course apply to other parenteral formulations, not only intravenous ones.

The intravenous preparations may take the form of large volume parenterals or of small volume parenterals, as described above.

In a specific embodiment, the present disclosure is directed to products, particularly kits, for producing a single-dose administration unit. The products (kits) may each contain both a first container having the active product (optionally combined with additives, for example anti-oxidant, preservative and, in some instances, tonicity agent) and a second container having the carrier/diluent (for example water, optionally containing one or more additives, for example tonicity agent). As examples of such products may be mentioned single and multi-chambered (e.g. dual-chamber) pre-filled syringes; exemplary pre-filled syringes are available from Vetter GmbH, Ravensburg, Germany. Such dual chamber syringes or binary syringes will have in one chamber a dry preparation including or consisting of the active product and in another chamber a suitable carrier or diluent such as described herein. The two chambers are joined in such a way that the solid and the liquid mix to form the final liquid medium.

The active product and the carrier are typically combined, for example in a mixer. After the formulation is mixed, it is preferably sterilized, such as with U.V. radiation. Once the formulation has been sterilized, it is ready to be injected or packaged for storage. It is envisaged, however, that the disclosed products will not be stored in liquid formulation but as dry solids, in order to prolong shelf life.

It will be understood from the aforegoing that there are provided pharmaceutical products comprising a disclosed product, suitable for reconstitution into an aqueous read-to-use parenteral formulation. One example is HDL hydroperoxide or LDL hydroperoxide for reconstitution as a liquid intravenous formulation (e.g. solution) containing a tonicity agent, particularly sodium chloride. The reconstitutable form used in a parenteral formulation may be a lyophilisate. The reconstituted liquid may be administered by injection or infusion.

Also provided are liquid formulations, e.g. solutions, comprising a liquid vehicle (typically water) and species which will result in in vivo lipoprotein peroxide upon administration of the formulation.

Further, the products of the invention may be used in combination with the target compounds. The products of the invention may be used in this way when they are formulated to have a predetermined release time. In this way, the period of activity of the target compound may be predetermined in that, just prior to expiration of the predetermined period of activity, a product of the present invention is released in the patient and the activity of the target compound is neutralised.

### EXAMPLES

Materials and Methods

Materials

Peptide boronate inhibitors were synthesised. Human citrated plasma was obtained from Hospital Services. Chromogenic substrates were obtained from Quadratech. Cholesteryl linoleate hydroperoxide and the lipid peroxide assay kit were purchased from Cayman Chemicals USA. Lipoproteins from bovine plasma (19 mg/ml) and lipoprotein fractions from human plasma; LDL (6.1 mg/ml), HDL (10.6 mg/ml) and VLDL (1.16 mg/ml) were obtained from Sigma Chemical Co. All other chemicals and proteins were obtained from Sigma Chemical Co.

Compound TRI 50b used in some of the examples is the pinacol ester of TRI 50c and is a prodrug for TRI 50c. TGN 255 is the monosodium salt of TRI 50c.

Assay of Peptide Boronate Inhibitors

Method A 3 μl TRI-50b (100 μg/ml) were added to 150 μl of sample in Buffer A (20 mM, Tris-HCl, 20 mM EACA, 0.1 M NaCl, 0.38% Na citrate, 0.02% Na azide, pH 7.4) and incubated at 37° C. At 1, 5, 20 and 60 minutes, 25 μl was removed and added to 50 μl S-2238 (200 μM) followed by 50 μl of human thrombin (9 nM) in Buffer B (0.1M Na phosphate, 0.2M NaCl, 0.5% PEG 6000, 0.02% Na azide pH 7.5). The thrombin activity was expressed as a percentage of the thrombin activity of the control incubation (without TRI-50b) at each time point. Results are given as the change in percentage activity (Δ %/min).

Method B Method as A except that the measurement was made at a single time point. The incubation period was 20 minutes with plasma samples and 1 minute with the purified lipoproteins. Results are given as the overall percentage activity relative to a control without inhibitor.

Method C. 20 μl of TRI-50b dissolved in DMSO was added to 313 μl of bovine lipoprotein (diluted 1 in 10 in Buffer A, see method A above) to achieve a range of final concentrations of 164, 333, 490, 660, 820, 990, 1150, 1320 and 1480 μM, respectively. Samples were removed at timed intervals and diluted 1 in 20 with buffer A. The concentration of TRI-50b in 25 μl of this mixture was determined as described in Method A. Residual TRI-50b was determined from a standards curve. The change in concentration of TRI-50b was calculated by subtracting the residual concentration from the starting concentration.

Ecarin Clotting Time

Neutralising activity of the product was determined by ecarin clotting time. The percentage of activity in a test sample was determined with respect to a control incubation, the control being performed in the absence of a boronic acid inhibitor of serine proteases

5 volumes of normal pooled plasma (20 donors stored at −80° C.) and 1 volume of bovine lipoprotein were mixed with 1 volume of TRI-50b in 50 Mm TRIS-HCl pH 7.5 of varying concentration to achieve a range of final concentrations in the range 0 to 235 μM. The mixture was incubated at 37° C. Sub-samples were taken at timed intervals and clotted by the addition of ecarin (14 volumes to 1 volumes of ecarin at 1.25 μg/ml in saline, 50 mM CaCl₂). The time to clot was determined by measuring OD₄₀₅ₙₘ nm every 6 second with an MDC Thermomax plate reader.

Gel Filtration

1 ml of plasma (pool of 5 donors) was adjusted to 1M NaCl and then run on to a 100×1.2 cm column of Sephacryl S-300 equilibrated in buffer A but 1M with respect to NaCl. Fractions of approximately 2 ml were collected, adjusted to 1 mM cupric sulphate and incubated at 37° C. overnight. Neutralising activity was determined using Method A.

### Example 1

### Copper Ion Mediated Oxidation of Plasma Generates an Activity that Destroys the Activity of TRI-50b

A well-established procedure for the oxidation of proteins is to incubate in the presence of Cu²⁺. [Puhl H, Waeg G, Esterbauer H. Methods to determine oxidation of low-density lipoproteins. Methods Enzymol 1994; 233:425-41.] Reactive oxygen species are generated capable of oxidising a wide range of amino acids within proteins [Berlett B S, Stadtman E R. Protein oxidation in ageing, disease, and oxidative stress. J Biol Chem 1997; 272:20313-6] and lipids contained within lipoprotein particles[Puhl H, Waeg G, Esterbauer H. Methods to determine oxidation of low-density lipoproteins. Methods Enzymol 1994; 233:425-41.].

FIG. 1 shows the effect of Cu 2+ ion on the ability of plasma to neutralise the activity of TRI-50b. Normal citrated plasma at different dilutions in Buffer A (Undiluted □-□, 1 in 4, ▴-▴; 1 in 16, ▾-▾; 1 in 64, ▪-▪) was mixed with increasing concentrations of cupric sulphate. After 24 hours, samples were analysed for neutralising activity according to Method A (Materials and Methods) as stated above. The copper treated plasma was observed to cause a rapid loss of inhibitory activity that was not a property of the untreated plasma (or of plasma treated with copper ion just before assay, data not shown). The optimum concentration of Cu²⁺ was observed to be from 0.1 to 1 mM depending on plasma dilution. The addition of 1 mM Cu²⁺ is standard procedure for the oxidation of lipoprotein as quoted within the literature.

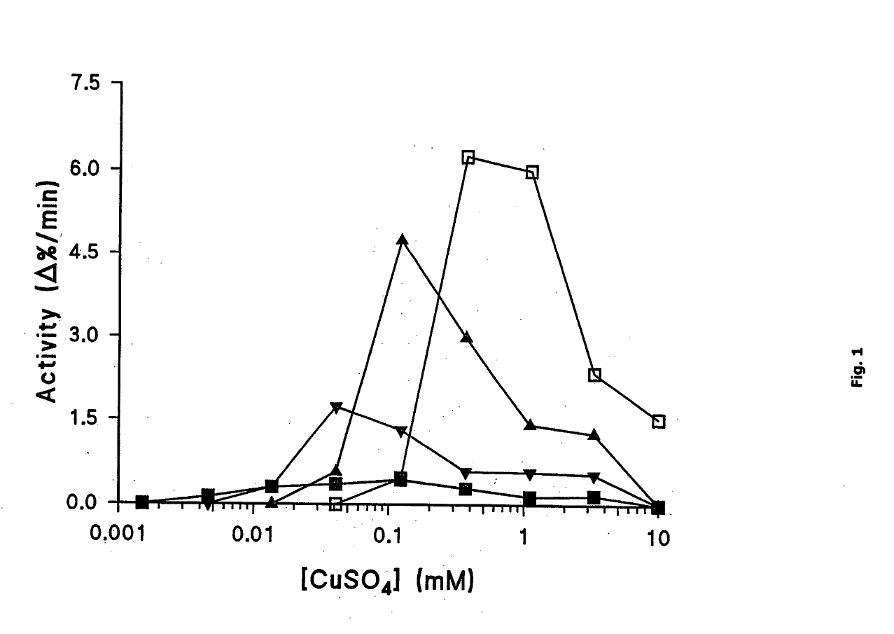

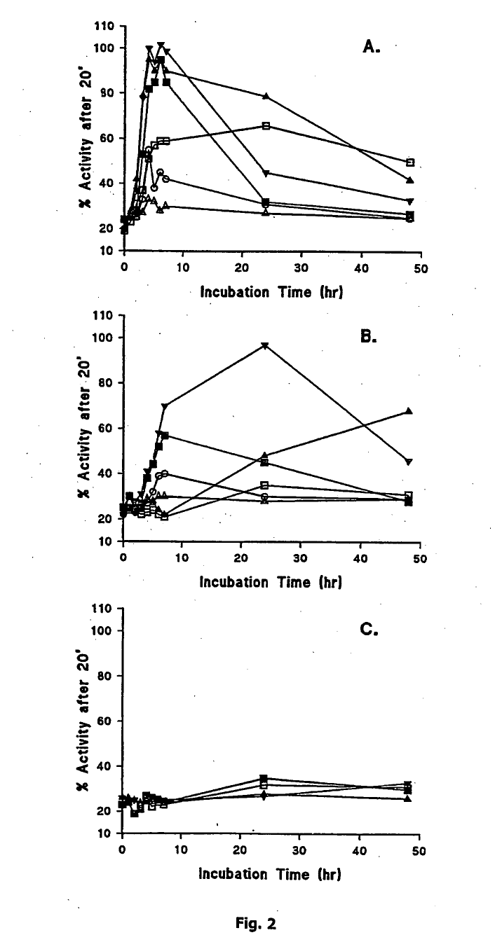

Experiments were then carried out in which copper was added at three different concentrations to different dilutions of plasma and the ability to neutralise TRI-50b was determined at timed intervals by Method B as stated above (Materials and Methods). FIG. 2 shows the effect of time of incubation with Cu²⁺ ion at three different concentrations on the ability of plasma to neutralise the activity of TRI-50b. Normal citrated plasma of varying dilutions Undiluted, □-□; 1 in 3, ▴-▴; 1 in 9, ▾-▾; 1 in 27, ▪-▪; 1 in 81, O-O; 1 in 243, Δ-Δ) was mixed with cupric sulphate. (Cupric sulphate concentration: A: 1.0 mM; B: 0.1 mM; C, 0.01 mM). As the results show (FIG. 2A-C), the speed at which neutralising activity was generated was directly proportional to the concentration of copper. An optimum concentration of Cu²⁺ was determined (1 mM) and also optimum dilution of plasma (1 in 9), generating the highest level of neutralising activity within 6 hours.

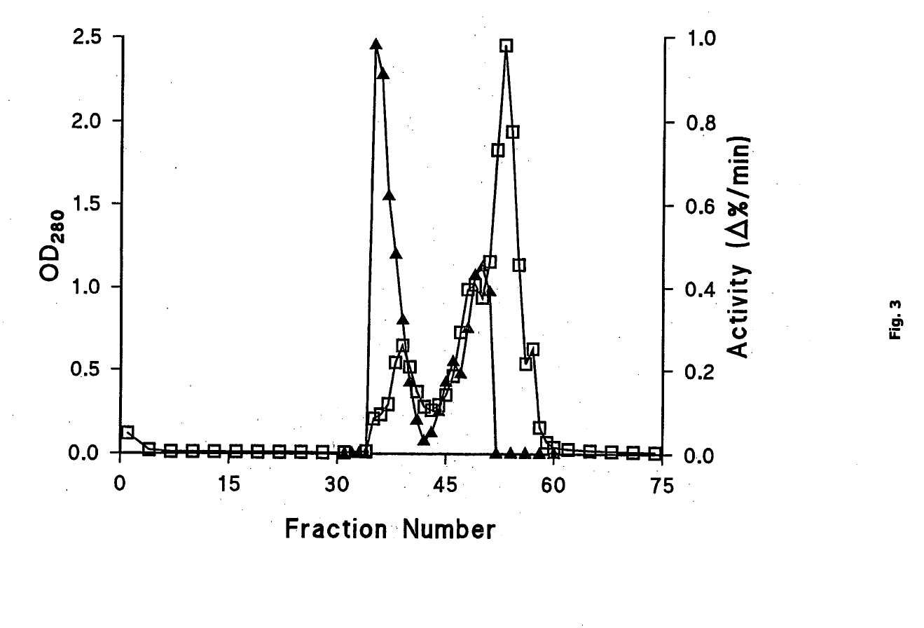

Plasma in which neutralising activity had been generated was then subjected to gel filtration. The principal components of the three main peaks obtained by gel filtration of plasma are well known to be albumin (60 kDa), globulins (120 kDa) and lipoproteins (>300 kDa). FIG. 3. shows the separation of the TRI-50b neutralising activity by gel filtration. Plasma was oxidised by incubation with 1.0 mM cupric sulphate for 24 hours. A sample was subjected to gel filtration on a 100×1.2 cm column of Sephacryl S-300 equilibrated with buffer B but 1M with respect to NaCl. 2 ml aliquots were collected and analysed for neutralising activity by Method A (Materials and Methods) □-□, OD 280 nm; ▴-▴, Neutralising activity. The results show a number of broad peaks of neutralising activity (FIG. 3). However the highest activity (and specific activity) was observed in the highest molecular weight fractions to elute from the column. These fractions will contain the lipoprotein particles (3×10⁶ kDa) which are the highest molecular weight components of plasma.

### Example 2

The Selection of Lipoprotein as the Source of the Highest Neutralising Activity.

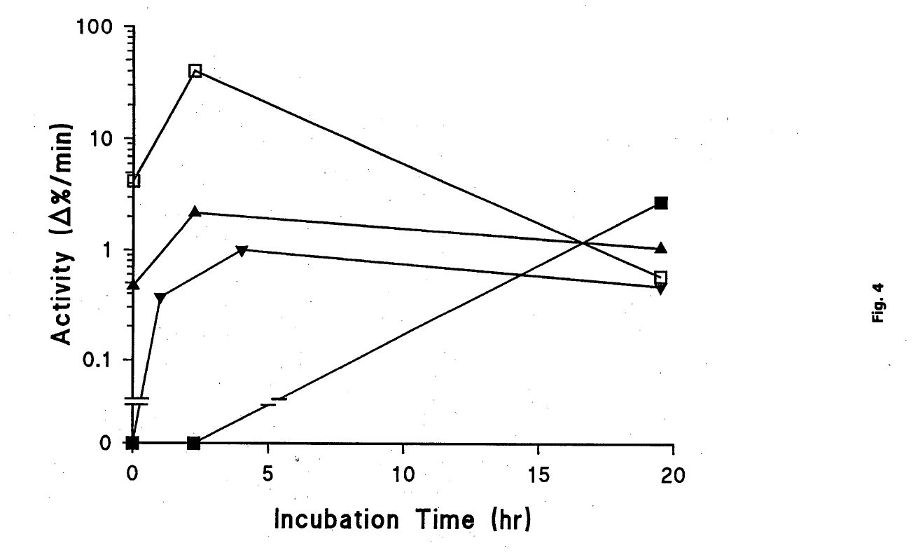

A number of purified lipoprotein fractions were purchased and tested for neutralising activity after incubation with 1 mM Cu²⁺. FIG. 4 shows the effect of incubation with Cu²⁺ ion on the ability of preparation of purified lipoprotein to neutralise the activity of TRI-50b. Lipoprotein fractions (Bovine lipoprotein, 1 in 30 dilution of stock Buffer □-□; LDL 1 in 3, ▴-▴; HDL 1 in 3, ▾-▾; VLDL 1 in 3, ▪-▪) were mixed with 1.0 mM cupric sulphate. At timed intervals, samples were analysed for neutralising activity by method A (Materials and Methods). The results show the highest activity obtained with a bovine fraction (BLp) that was quite active even in the absence of Cu²⁺ (t=0). Of the human lipoprotein fractions tested LDL>HDL>VLDL (FIG. 4).

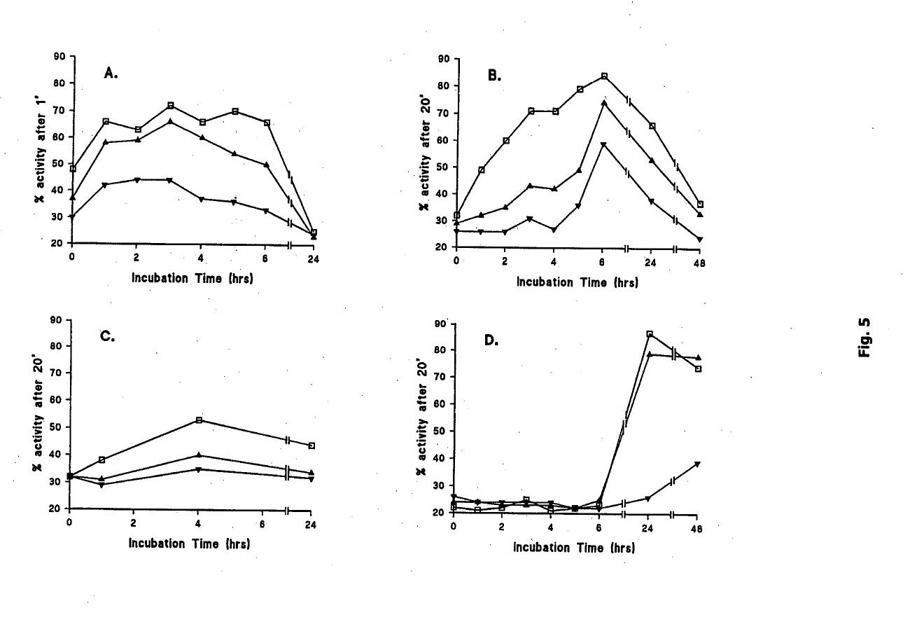

The increase in neutralising activity was also measured at timed intervals up to 24 hours and at three different concentrations of each lipoprotein. FIG. 5 shows the effect of time of incubation with Cu²⁺ ion on the ability of varying dilutions of purified lipoprotein to neutralise the activity of TRI-50b. Lipoprotein fractions of varying dilution (Lowest dilution, □-□, ▴-▴ to highest dilution, ▾-▾) were mixed with 1.0 mM cupric sulphate. At timed intervals, samples were analysed for neutralising activity by Method B (Materials and Methods). Lipoprotein source: A, Bovine lipoprotein (BLp) 1 in 10, 1 in 30, 1 in 90 dilution of stock; B; Human LDL, 1 in 4, 1 in 12, 1 in 36; C; Human HDL, 1 in 4, 1 in 12, 1 in 36; D, Human VLDL, 1 in 3, 1 in 9, 1 in 27. As the results show (FIGS. 5A-D) each of the lipoprotein fractions reached maximum activity after 6 hours and declined thereafter apart from VLDL. VLDL activity was not present at 6 hours but was observed in the 24 and 48 hour samples (FIG. 5D).

### Example 3

Neutralisation of TRI-50b by Peroxides from Oxidised Plasma

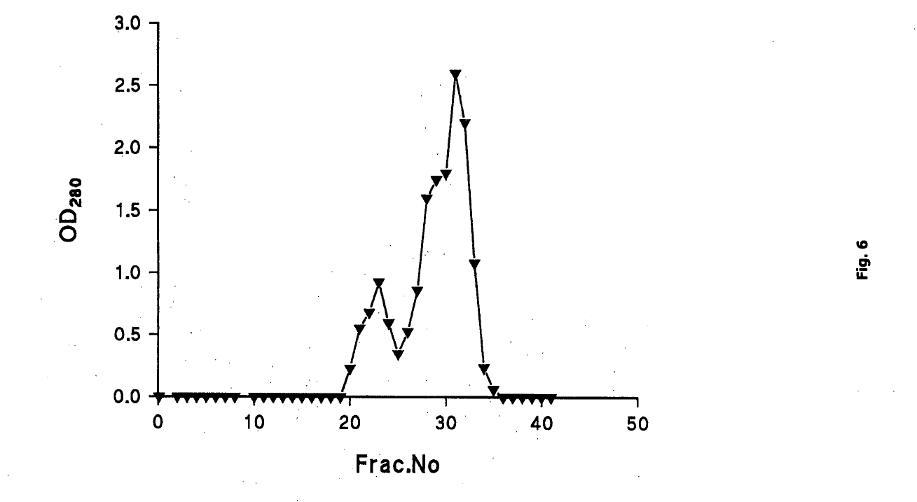

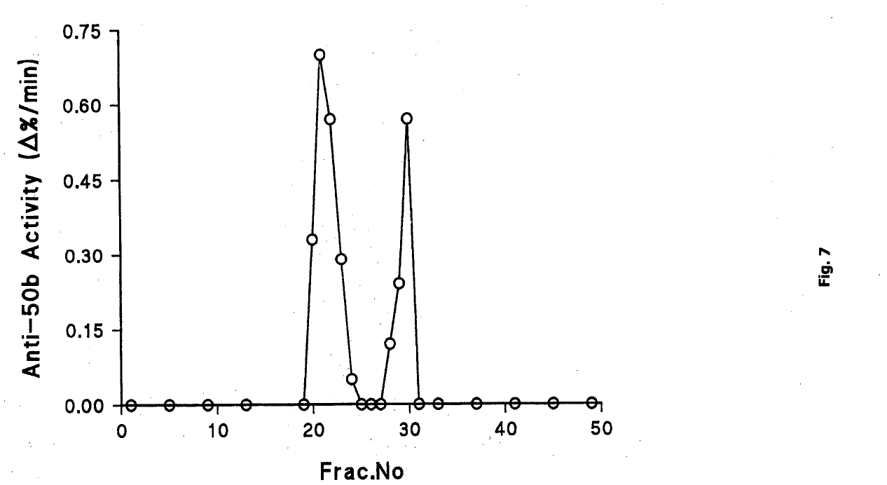

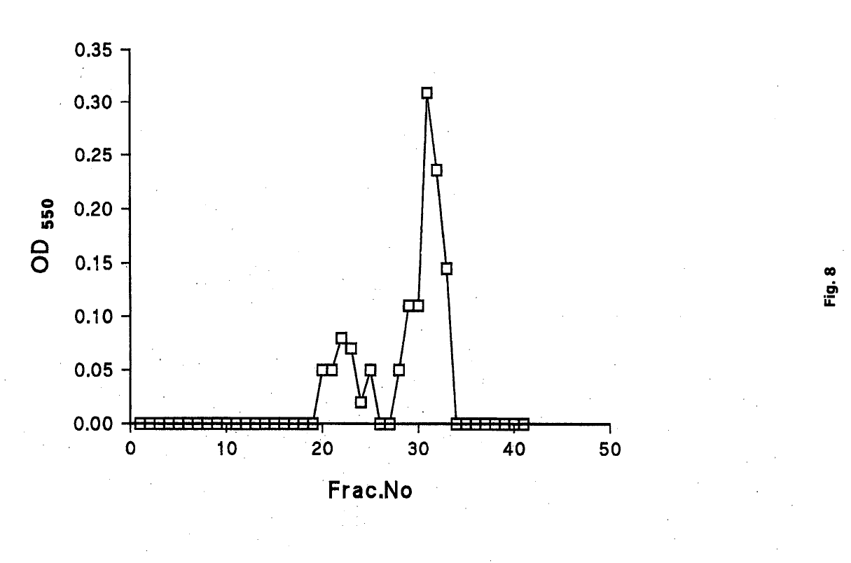

Citrated plasma was oxidised by incubation with Cu²⁺ (1 Mm) for 24 hours. The plasma was then chromatographed over Sephacryl s-300 (1.2×90 cm equilibrated with Buffer A) at room temperature. 3 ml fractions were collected and analysed for protein content. The results are shown in the graph of Figure of protein content (OD 280) of fractions prepared from oxidised plasma by gel filtration (FIG. 6). TRI-50b neutralising activity of fractions prepared from oxidised plasma were assessed by Method A (FIG. 7). In addition, peroxide content of the fraction was measured by ferrous oxidation in a xylenol orange assay (Nourooz-Zadeh et al Measurement of plasma hydroperoxide concentrations by ferrous oxidation-xylenol orange assay in conjunction with triphenylphosphine. Anal Biochem 1994, 220 (2) 403-409) (FIG. 8).

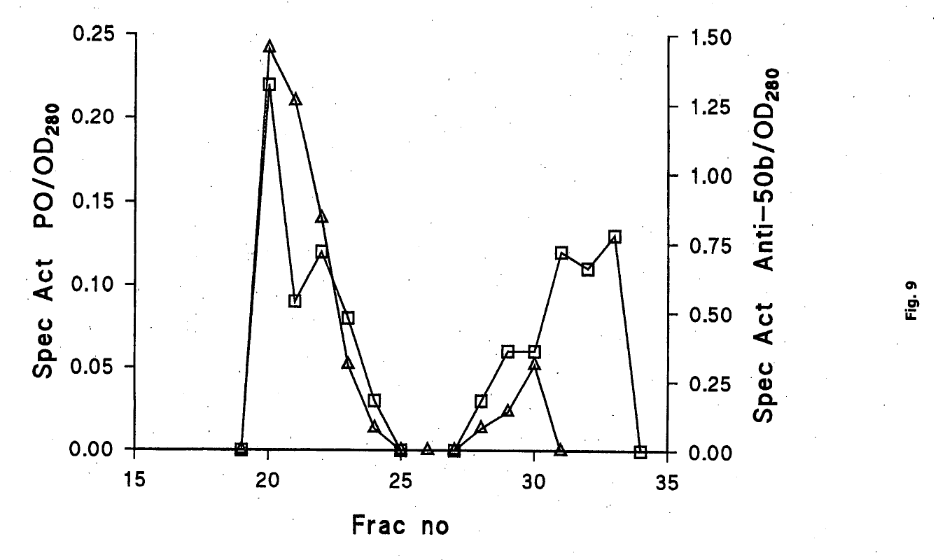

Results were expressed as specific activity, that is peroxide/weight protein and neutralising activity per weight protein. FIG. 9 shows a graph of the correlation between peroxide content of fractions of oxidised plasma □-□ and the neutralisation of TRI-50b A-A. The highest neutralising activity was found in the highest molecular weight fraction which is associated with lipoproteins. Interestingly the highest peak of peroxide activity (FIG. 8) had no activity as a neutraliser of TRI-50b. We believe that the best candidate as a neutraliser of TRI-50b is the lipoprotein fraction because of its high specific activity.

### Example 4

Neutralisation by Lipoproteins

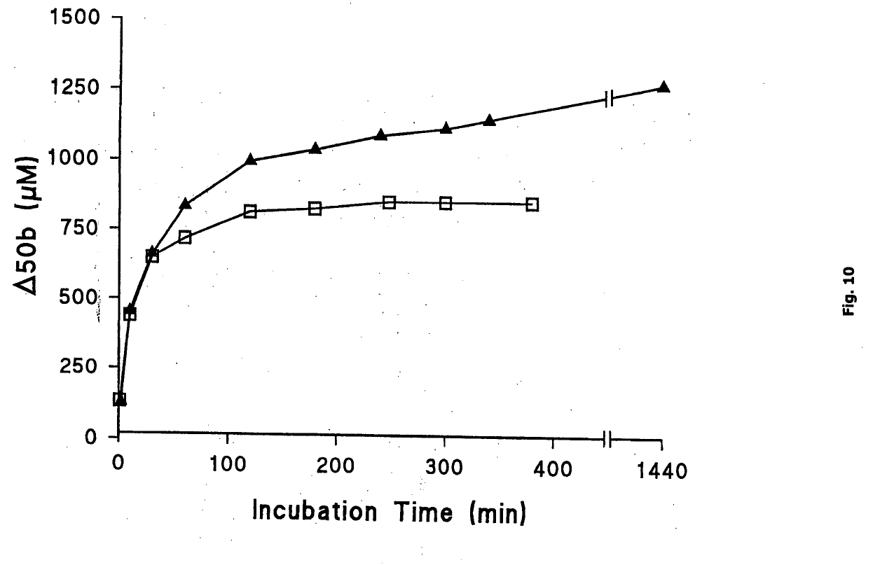

FIG. 10 shows the determination of the total neutralising capacity of bovine lipoprotein fraction. BLp was incubated at 37° C. in the presence or absence of 1 mM cupric sulphate, Samples were taken at timed intervals and added to varying concentrations of TRI-50b up to 1.5 mM. The residual TRI-50 b was measured by Method C (Materials and Methods). —Cu²⁺ □-□; +Cu²⁺ ▴-▴. In FIG. 10 are shown the results of an experiment in which the total mass of neutralising activity of bovine lipoprotein was determined. Increasing concentrations of TRI-50b up to 1.5 mM were added to a ten-fold dilution of the BLp fraction (OD280 nm=4.0). A rapid neutralisation of TRI-50b was observed reaching a maximum beyond which the neutralising capacity was totally exhausted. In the absence of Cu²⁺, the maximum mass of TRI-50b that was neutralised was equivalent to 0.8 mM. The concentration of BLp in the mixture was 1.9 g/l or 0.0038 mM. Thus around 200 moles of TRI-50b were neutralised per mole of lipoprotein. In the presence of Cu²⁺, the maximum mass of TRI-50b that was neutralised was higher and equivalent to 320 moles/mole.

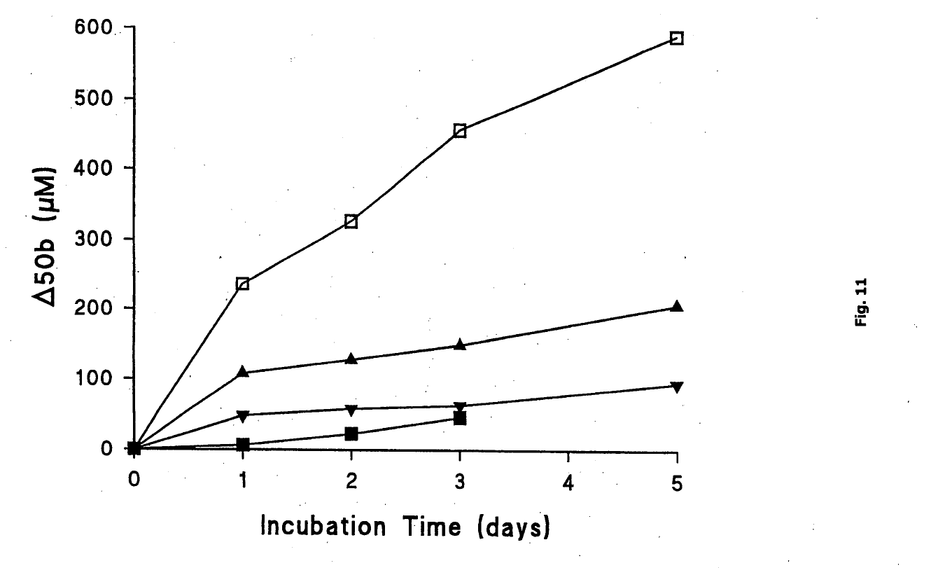

A similar experiment was carried out with dilutions of plasma (FIG. 11). FIG. 11 shows the determination of the total neutralising capacity of plasma following oxidation. Plasma at varying dilutions was incubated at 37° C. in the presence of 1 mM cupric sulphate. Samples were taken at timed intervals and added to varying concentrations of TRI-50b up to 1.5 mM. The residual TRI-50 b was measured by Method C (Materials and Methods). 1 in 3, □-□; 1 in 6, ▴-▴; 1 in 12, ▾-▾; 1 in 3 without Cu²⁺, ▪-▪. Assuming that the neutralising activity arises principally from the lipoprotein fraction of plasma (around 0.016 mM), then 120 moles/mole of lipoprotein fraction separated from human plasma are neutralised at the 1 in 3 dilution, 80 at the 1 in 3 dilution and 80 at the 1 in 12 dilution.

### Example 5

Neutralisation by Cholesteryl Linoleate Hydroperoxide

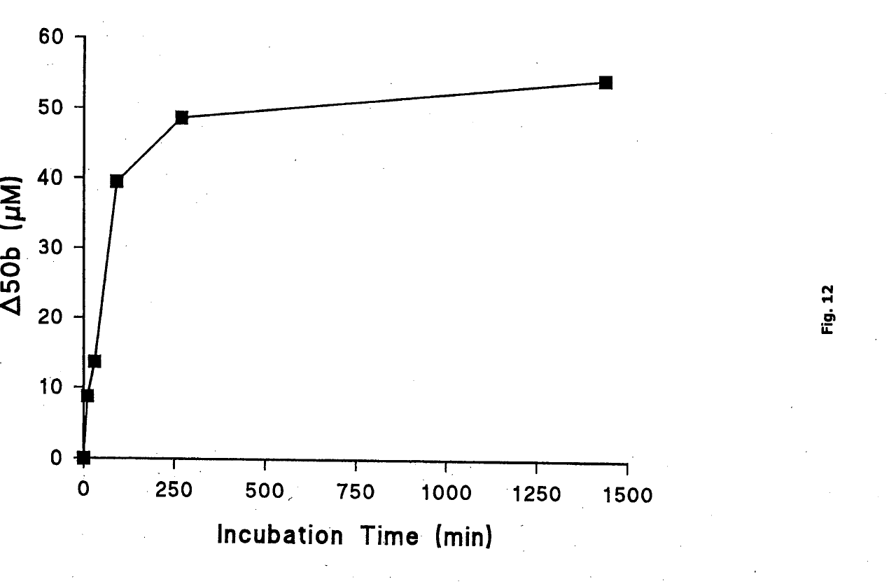

The low-density lipoprotein particle contains a single molecule of a 500 kDa protein and 1500 molecules of esterified cholesterol. Because this value is in general agreement with the high number of molecules of TRI-50b neutralised per mole of lipoprotein, it was investigated if the oxidised form of cholesterol ester was responsible for the neutralising activity. FIG. 12 shows the neutralisation of TRI-50b by cholesteryl linoleate hydroperoxide. Cholesteryl linoleate hydroperoxide (44 μM) was incubated with TRI-50b in the range 0 to 4.9 mM. Samples were taken at timed intervals and the residual TRI-50b concentration was determined by Method C (Materials and Methods). When pure cholesteryl linoleate hydroperoxide at a concentration of 44 μM was mixed with TRI-50b, a high rate of neutralisation was observed with an equivalence of approximately 1 mole/mole (FIG. 12).

### Example 6

Neutralisation of a Number of Peptide Boronates by Lipoprotein

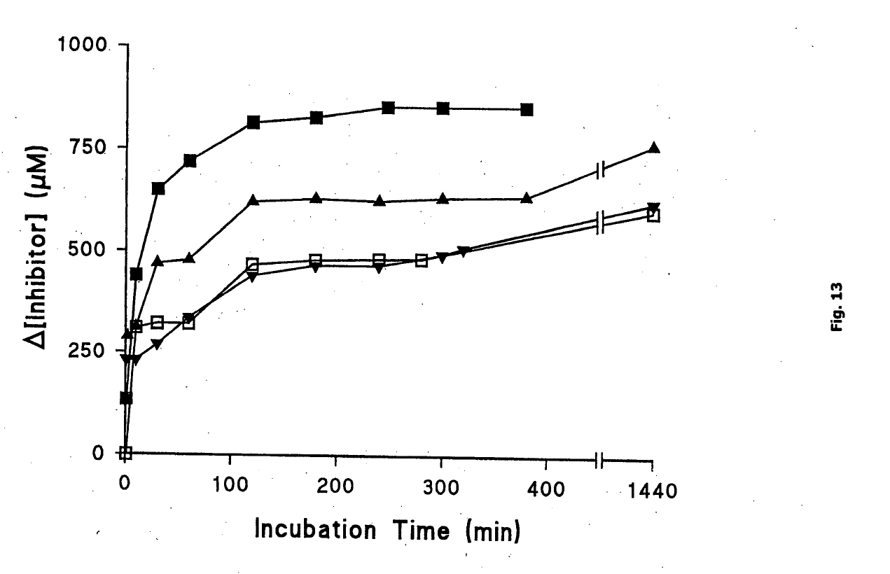

In FIG. 13 are shown the observations made upon the neutralisation of a number of other peptide boronates by BLp added in the absence of Cu²⁺, namely TRI-30, Z-D-Dpa-Pro-BoroMbg-Opin, TRI-26, Z-D-βNal-Pro-BoroIrg-Opin; TRI-142, Moc-D,L-Dpa-Pro-BoroMpg-Opinac. BLp was incubated at 37° C. in the presence or absence of 1 mM cupric sulphate. Samples were taken at timed intervals and added to varying dilutions of TRI-50b, ▪-▪; TRI-30, ▴-▴; TRI-142, ▾-▾ and TRI-26, □-□ up to 1.5 mM. The residual concentration of boronate was determined by Method C (Materials and Methods). The results show that all three were neutralised by the lipoprotein although the maximum number of molecules destroyed varied according to the structure, namely TRI-30 around 80 moles/mole and TRI-26 and TRI-142 around 65 moles/mole.

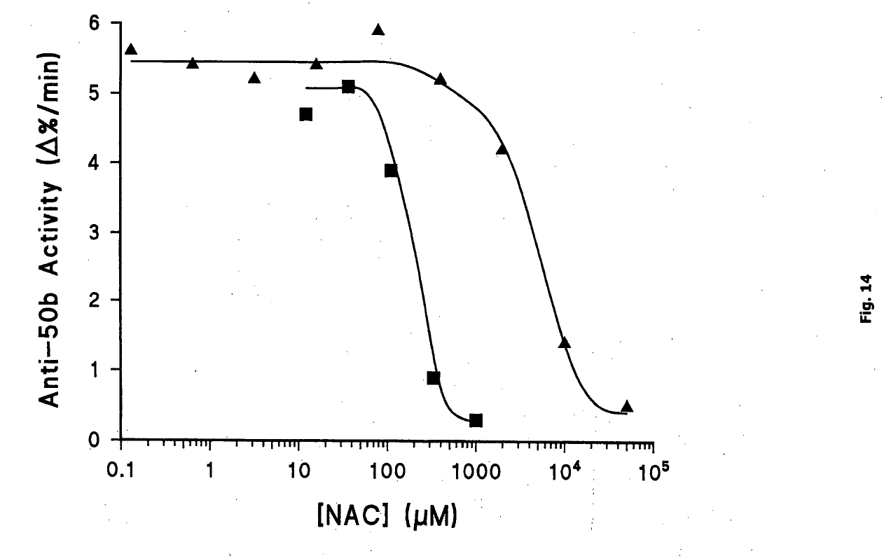

If lipid peroxides are involved in the inactivation then, the addition of n-acetyl-cysteine (NAC), a powerful anti-oxidant should reduce the neutralising activity. (NAC has been used historically to mop up reactive oxygen species). FIG. 14 shows the effect of N-acetyl cysteine upon the TRI-50 neutralising activity of bovine lipoprotein and oxidised plasma. Plasma (1 in 10 dilution in buffer A) was incubated with 1.0 mM cupric sulphate for 4 hours. Increasing concentrations of n-acetyl cysteine were then added and also to BLp (1 in 10 dilution in buffer A). The neutralisation of TRI-50b was measured by Method B (Materials and Methods). Plasma, ▪-▪, BLp ▴-▴. A reduction in neutralising activity, to approximately base-line levels was observed when increasing concentrations of NAC were added to plasma or the bovine lipoprotein fraction (FIG. 14). The IC50 for quenching of the neutralising activity of oxidised plasma was around 100 μM and with BLp around 2 mM NAC.

### Example 7

Reduction of Boropeptide Ki

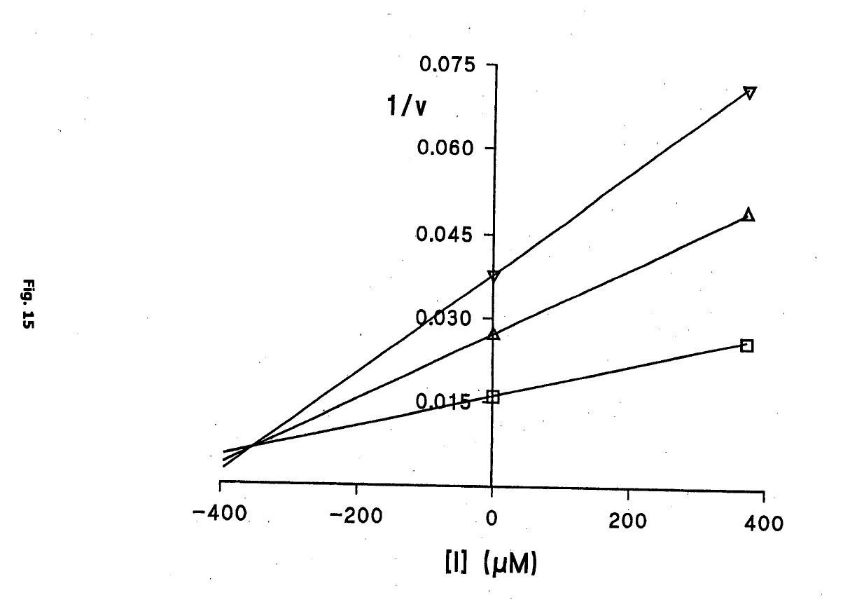

The loss of activity of boropeptides when contacted with peroxide compounds was apparently due to the removal of the boronic acid moiety (as was observed upon treatment of TRI-50b with hydrogen peroxide). The boron provides at least four orders of binding energy to the peptide sequence because of the formation of a transition state-like adduct with the active site serine. FIG. 15 shows the determination of the Ki of TRI-50b subjected to treatment with hydrogen peroxide. 1 volume TRI-50b (15 mg/ml in DMSO) was added slowly to 10 volumes of hydrogen peroxide (3.0%) for 2 hours at RT. The Ki for inhibition of thrombin activity was carried out using Dixon plots. The initial rate of cleavage of S2302 (a chromogenic substrate for thrombin) by thrombin (28 nM) was determined at the different concentrations of substrate in the presence of varying concentrations of the oxidised inhibitor. This loss of binding energy was observed as a 20,000 fold increase in the Ki against thrombin (FIG. 15). A range of peptide boronate structures have been shown to be susceptible to inactivation by the bovine lipoprotein for which reason, it is probable that this approach could be used to control the activity of all peptide boronates. As the results show, the neutralisation is rapid and effective when the lipoprotein is added to TRI-50b dissolved in plasma.

Conclusion

It is envisaged that therapeutic levels of boropeptides of the tripeptide Cbz-Phe-Pro-Mpg in plasma will preferably be no greater than 0.5 μM (TRI 50b is converted to TRI 50c in plasma). From our preliminary studies of the concentration dependency of the rate of neutralisation, we estimate a blood level 0.5 μM would be sufficient to cause rapid reversal of the activity of that level of TRI-50c. This would require the infusion of 0.75 g of BLp as supplied by Sigma equivalent to around 40 ml of the product. The use of this product therefore seems a viable therapeutic proposition to reverse therapy by TRI 50c or a TRI 50c salt or prodrug (e.g. TRI 50b). Furthermore, there is good reason to believe that more potent preparations can be also developed (with higher % oxidised lipid, for example) that would reduce the amount of protein that needed to be infused. It can also be predicted that other organoboronate compounds could be neutralised using this approach.

Thus, boronate inhibitors [Groziak M P. Boron therapeutics on the horizon. Am J Ther 2001; 8:321-8) have been described as proteasome inhibitors [Shah S A, Potter M W, McDade T P, et al. 26S Proteasome inhibition induces apoptosis and limits growth of human pancreatic cancer. J Cell Biochem 2001; 82:110-22; Hideshima T, Richardson P, Chauhan D, et al. The proteasome inhibitor PS-341 inhibits growth, induces apoptosis, and overcomes drug resistance in human multiple myeloma cells. Cancer Res 2001; 61:3071-6.] beta-lactamase inhibitors [Usher K C, Blaszczak L C, Weston G S, Shoichet B K, Remington S]. Three-dimensional structure of AmpC beta-lactamase from Escherichia coli bound to a transition-state analogue: possible implications for the oxyanion hypothesis and for inhibitor design. Biochemistry 1998; 37:16082-92; Tondi D, Powers R A, Caselli E, et al. Structure-based design and in-parallel synthesis of inhibitors of AmpC beta-lactamase. Chem Biol 2001; 8:593-611.], dipeptidyl peptidase inhibitors [Tanaka S, Murakami T, Horikawa H, Sugiura M, Kawashima K, Sugita T. Suppression of arthritis by the inhibitors of dipeptidyl peptidase IV. Int J Immunopharmacol 1997; 19:15-24], inositol triphosphate receptor modulators, [Ma H T, Patterson R L, van Rossum D B, Birnbaumer L, Mikoshiba K, Gill D L. Requirement of the inositol trisphosphate receptor for activation of store-operated Ca2+ channels. Science 2000; 287:1647-51: Gregory R B, Rychkov G, Barritt G J. Evidence that 2-aminoethyl diphenylborate is a novel inhibitor of store-operated Ca²⁺ channels in liver cells, and acts through a mechanism which does not involve inositol trisphosphate receptors. Biochem J 2001; 354:285-90] antibacterials [Levy C W, Baldock C, Wallace A J, et al. A study of the structure-activity relationship for diazaborine inhibition of Escherichia coli enoyl-ACP reductase. J Mol Biol 2001; 309:171-80.] and antiestrogens [Endo Y, Yoshimi T, Yamakoshi Y. New estrogenic antagonists bearing dicarba-closo-dodecaborane as a hydrophobic pharmacophore. Chem Pharm Bull (Tokyo) 2000; 48:312-4.]. It may be predicted that these compounds will also be susceptible to control by this approach.

### Example 8

Neutralisation of TRI-50b in Human Plasma by the Addition of the Bovine Lipoprotein Fraction

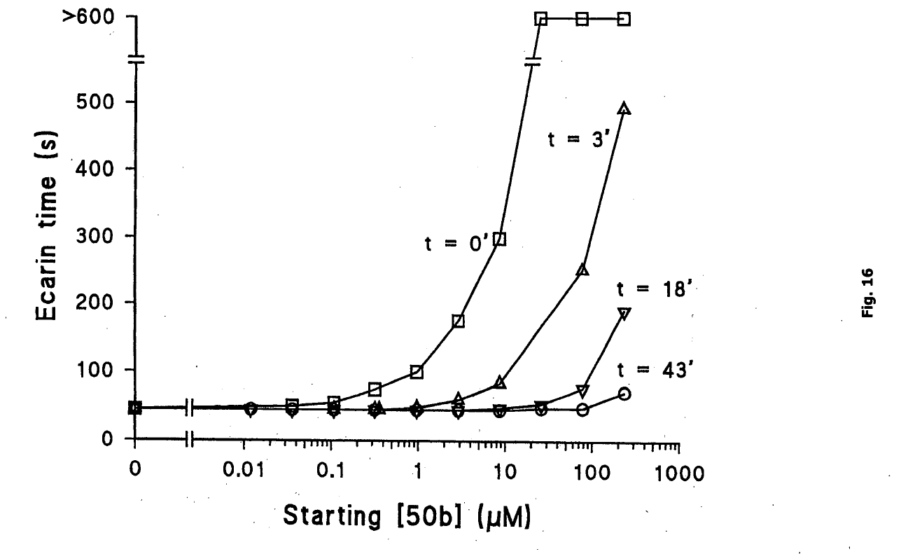

FIG. 16 shows the neutralisation of TRI-50b in human plasma by the addition of bovine lipoprotein. TRI-50b was added at increasing concentrations to human pooled plasma at 37° C. At t=0, BLp was added (final concentration around 10 μM) and the ecarin clotting time was measured at timed intervals. A time dependent decrease in the ecarin clotting time of plasma containing increasing concentrations of TRI-50b following the addition of the bovine lipoprotein fraction was observed. Within 3 minutes the activity of 1 μM TRI-50b has been completely neutralised. After 40 minutes, 100 μM TRI-50b has been destroyed.

Lipid hydroperoxides are the primary stable products of lipid peroxidation [Yamamoto Y. Fate of lipid hydroperoxides in blood plasma. Free Radic Res 2000; 33:795-800]. The major forms generated in vitro are cholesteryl ester hydroperoxide (CE-OOH) and phosphatidyl hydroperoxide (PC—OOH). In vivo PC—OOH is reduced by plasma glutathione peroxidase as well as by conversion to CE-OOH by lecithin: cholesterol acyltransferase present in high-density lipoprotein. The principal form of CE-OOH found in plasma is cholesteryl linoleate hydroperoxide, which we have observed is capable of neutralising TRI-50b. This evidence together with the finding that oxidised lipoprotein is capable of neutralising more than 200 molecules of TRI-50b per molecule of lipoprotein is evidence that lipid peroxides are the principal source of the neutralising activity in the lipoprotein fraction of plasma.

### Example 9

Determination of the Structure of TRI-50b Following Treatment with Hydrogen Peroxide.

1 volume of TRI-50b (100 mg was dissolved in DMSO) was added to 10 volumes of H₂O₂. After 2 hours at RT, the sample was concentrated to dryness, diluted at ˜400 mg/ml in acetonitrile and loaded on a Phenomenex Luna C18 column 21.2×250 mm, 5 μM column, eluting at 5 mlmin⁻¹ (Solvent A: water Solvent B: acetonitrile). Samples were collected, pooled according to retention time freeze dried and prepared for analysis in deuterochloroform for NMR and acetonitrile for mass spectrometry.

NMR Analysis Using a Bruker Avance 400 spectrometer, sample in CDCl3, spectrum collected 1H NMR 400.1 MHz, collected over 256 scans.

| | Sample | | | TRI 50c | | |
|---|---|---|---|---|---|---|
| Shift (δ) | Integration | multiplicity | assignment | Integration | multiplicity | Assignment |
| 8.6 | 0.32H | d | | N/O | | |
| 7.15 | 10.5H | m | 2xPh | 10.6 | m | 2xPh |
| 5.9 | 0.3H | d | | N/O | | |
| 5.5 | 1.3H | m | NH | N/O | | |
| 5.1 | 2.0H | Q | OCH2Ph | 2H | m | OCH₂Ph |
| 4.5-4.1 | 1.6H | m | Phe C1, Pro | 2H | m | Phe C1, Pro |
| | | | C1 | | | C1 |
| 3.6 | 1.35H | m | Pro C4 1xH | 1H | m | Pro C4 1xH |
| 3.2 | 2H | | CH₂CH₂Ph | 2H | m | CH₂Ome |
| 2.95 | N/O | | | 3H | m | Ome |
| 2.9 | N/O | | | 2H | m | CH₂CH₂Ph |
| 2.6 | N/O | | | 1H | m | CHB |
| 2.51 | 1.88H | m | Pro C4 | 1H | m | Pro C4 |
| | | | 1xCH | | | 1xCH |
| 2.0 | 0.9H | M | Pro C2 | 1H | m | Pro C2 |
| | | | 1xCH | | | 1xCH |
| 1.8 | 2.18H | M | Pro C2 | 1H | m | Pro C2 |
| | | | 1xCH | | | 1xCH |
| 1.6 | 1.7H | M | Pro C3, | 4H | m | Pro C3, |
| | | | | | | CH₂CH₂ |
| 1.1 | N/O | | pinacol | 12H | s | Pinacol |

Legend:
N/O means not observed

HPLC Analysis and Mass Spectrometry Analysis of Sample 27114:

(TSP HPLC system, Phenomenex Luna column, 4×250 mm, flow rate 1.5 ml/min, detection at 254 nM, sample 27114 1 mg/ml in ethanol, injection of 100 μl, gradient 5-100% acetonitrile over 30 minutes). Peaks collected and analysed by electrospray mass spectrometry (Finnigan SSQ spectrometer) with methanol as eluant:

| Peak retention | weight collected | molecular ion | |
|---|---|---|---|
| time (min) | (mg) | observed | Assignment |
| 17.6 | trace | none | None |
| 18.3 | trace | none | None |
| 29.6 | 0.05 | 813 (2M + Na), 418 | Dipeptide |
| | | (M + na) | amide |
| 33 | trace | 536 | None |
| 541 | trace | 541.5 | None |

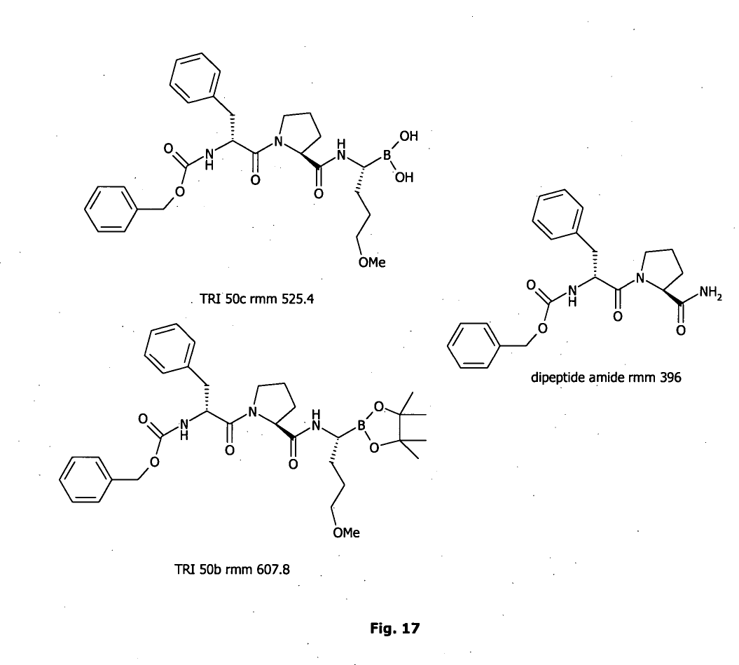

The structures of TRI 50b, TRI 50c and the dipeptide amide are shown in FIG. 17.

Conclusion:

The major product from oxidation of TRI 50b is the dipeptide amide.

### Example 10

Neutralising Activity of Activated High Denity Lipoprotein

Method A

50 μl thrombin (33.3 ng/ml in assay buffer) and 20 μl vehicle or compound solution were added to 110 μl assay buffer (100 mM Na orthophosphate (80% Na₂HPO₄ and 20% NaH₂PO₄), 200 mM NaCl, 0.5% PEG 6000, 0.02% Na azide, pH 7.5) and incubated for 5 minutes at 37° C. After the incubation period 20 μl, of thrombin substrate (50 μM, S2238) was added and changes in Vmax was monitored on a plate reader for 10 minutes using a wavelength of 405 nm at 37° C. Results are expressed as percentage change in thrombin activity.

Method B

High density lipoprotein (HDL, Merck Biosciences, UK) was activated by incubating with copper sulphate (CuSO₄, Sigma UK) at 37° C. for 2.5 hours.

Method C

50 μl thrombin (33.3 ng/ml in assay buffer) and 20 μl TRI 50c (1 to 10 μM) were added to 10 μl assay buffer (100 mM Na orthophosphate (80% Na₂HPO₄ and 20% NaH₂PO₄), 200 mM NaCl, 0.5% PEG 6000, 0.02% Na azide, pH 7.5) and incubated for 5 minutes at 37° C. After the incubation period 100 μl of activated HDL (Method B) was added and further incubated for 30 minutes at 37° C. After the second incubation period, 20 μl of thrombin substrate (50 μM, S2238) was added and changes in Vmax was monitored on a plate reader for 10 minutes using a wavelength of 405 nm at 37° C. Results were expressed as percentage reversal of TRI 50c inhibition.

Method D

Ecarin was obtained from HTI (USA) whilst the normal human plasma was purchased from Precision Biologics via Alpha Laboratories (UK). Rat plasma was purchased from Harlan laboratories, UK.

TRI 50c was diluted in an assay buffer (100 mM sodium orthophoshate, 200 mM NaCl, 0.5% PEG 6000 and 0.02% sodium azide, pH 7.5) and incubated with normal human pooled or rat plasma (80% final) for 5 minutes at 37° C. Ecarin (100 μg/ml final) was added to the incubating solution. The clotting times were determined using a plate reader (Versamax, Molecular Devices Corporation) at 405 nm for up to 10 minutes at 37° C.

Method E

HDL was oxidised with CuSO4 (500 μM) for 2.5 hr at 37° C. Neutalising activity was evaluated in the thrombin assay using 1 and 10 μM TRI 50c for up to 2 months. Oxidised HDL was maintained at 4° C. for the duration of the study.

Results

1. Copper Sulphate and Thrombin Activity

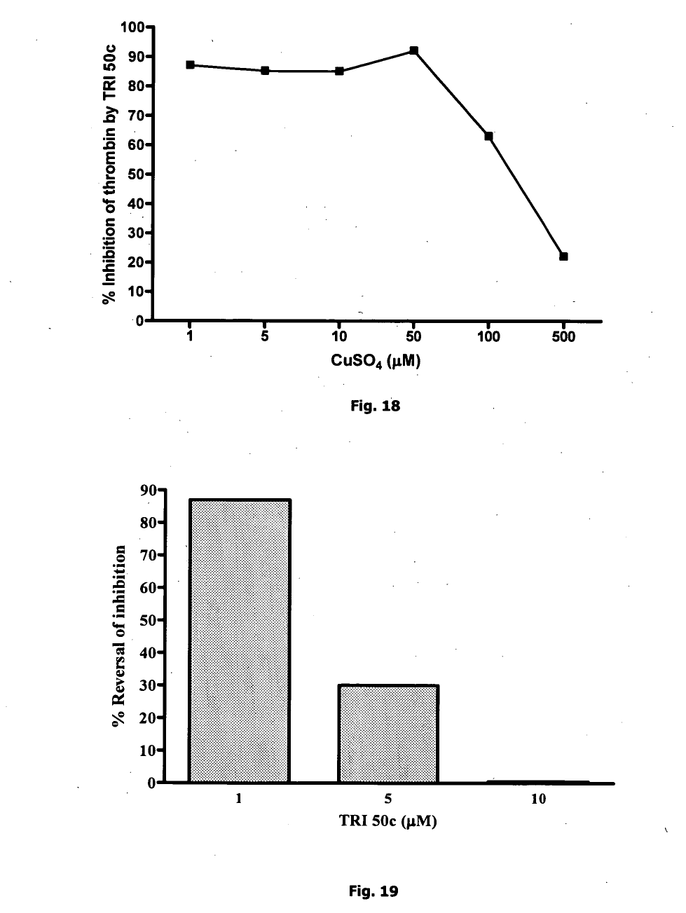

Thrombin assay was established as outlined in Method A and the effects of CuSO₄ concentrations (1, 5, 10, 50, 100 and 500 μM) were evaluated. FIG. 18 shows the effects of CuSO₄ on thrombin activity. CuSO₄ at concentrations greater than 50 μM (final concentration) began to reduce the inhibitory activity of TRI 50c.

2. Activated HDL and TRI 50c Activity

HDL was activated e.g. oxidised, with CuSO₄ (750 μM) as outlined in Method B and used in Method C. FIG. 19. shows the effects of activated HDL (1.5 mg/ml final) on percentage reversal of TRI 50c inhibition. Activated HDL produced 80-90% reversal of 1 μM TRI 50c inhibitory activity. At 5 μM TRI 50c concentration, the percentage reversal was reduced to approximately 20-30% whilst at 10 μM TRI 50c there was no reversal of activity.

3. Activated and Non-Activated HDL on TRI 50c Activity

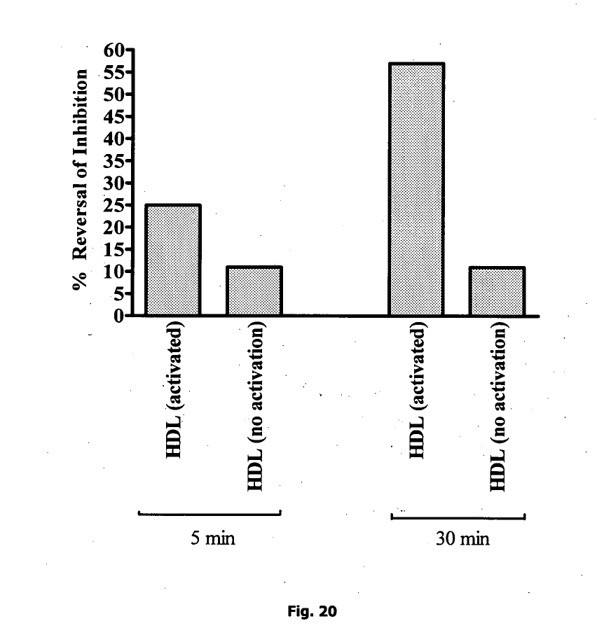

HDL was activated e.g. oxidised, with CuSO₄ (750 μM) as outlined in Method B whilst for non-activated HDL was used in the assay with the addition of CUSO₄ (50 μM final) in the assay. Both HDL preparations (1.5 mg/ml final) were used in Method C and evaluated at two time points namely at 5 and 30 minutes post addition of activated/non-activated HDL to the assay. FIG. 20 shows that activated HDL can reverse the inhibitory activity of 1 μM TRI 50c after 5 minutes (˜25%) whilst at 30 minutes the percentage reversal was approximately 55%. In contrast, non-activated HDL displayed minimal activity at both time points.

4. Copper Sulphate and Activation of HDL

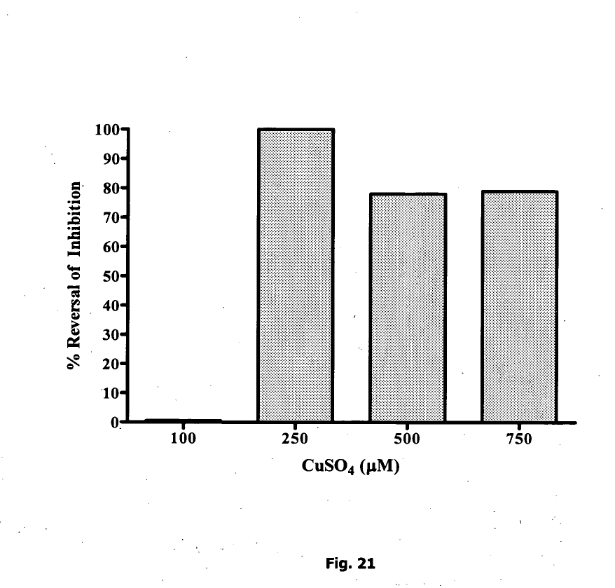

In these experiments, HDL was also activated with 100, 250, 500 and 750 μM CuSO₄ for 2.5 hours using Method B. FIG. 21 shows that activating HDL (1.5 mg/ml final) with 250 to 750 μM CuSO₄ produced approximately 70-100% reversal of 1 μM TRI 50c inhibition after 30 minutes incubation. However, HDL activation with 100 μM CuSO₄ produced no reversal of TRI 50c activity.

5. Copper Sulphate and High Concentrations of HDL

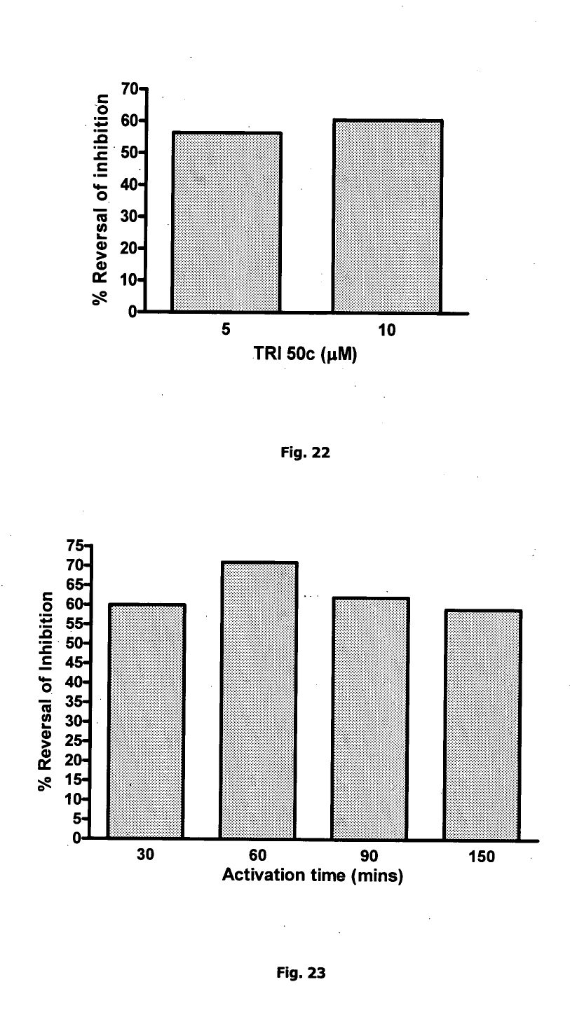

In these experiments, HDL was activated with 250 μM CuSO₄ for 2.5 hours using Method B and subsequently used in Method C. FIG. 22 shows that CuSO₄ (50 μM final) activated HDL (4.57 mg/ml final) produced approximately 55-60% reversal of 5 and 10 μM TRI 50c inhibition after 30 minutes incubation.

6. Activation Time Course Studies with HDL

HDL was activated with CuSO₄ (250 μM) using Method B. However, the HDL was activated for 30, 60, 90 and 150 minutes and used in Method C. FIG. 23 shows the percentage reversal of 5 μM TRI 50c inhibition by activated HDL (4.5 mg/ml final). At all time points, activated HDL produced between 55-70% reversal of TRI 50c activity.

7. Oxidised HDL and Ecarin Clotting Times (ECT) in Human Plasma Studies

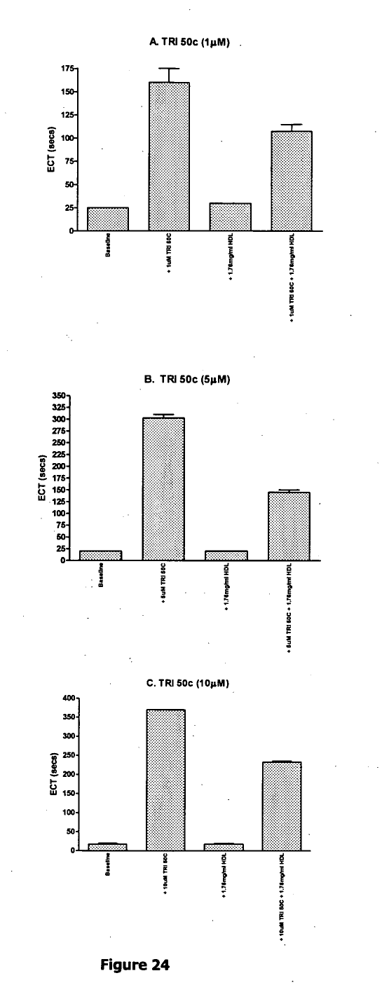

TRI 50c (1-10 μM) produced a concentration dependent prolongation of ECT in the presence of human plasma (FIGS. 24A-C). Oxidised HDL produced 40, 56 and 39% reversible of TRI 50c activity at 1, 5 and 10 μM respectively.

8. ECT and Fresh Rat Plasma

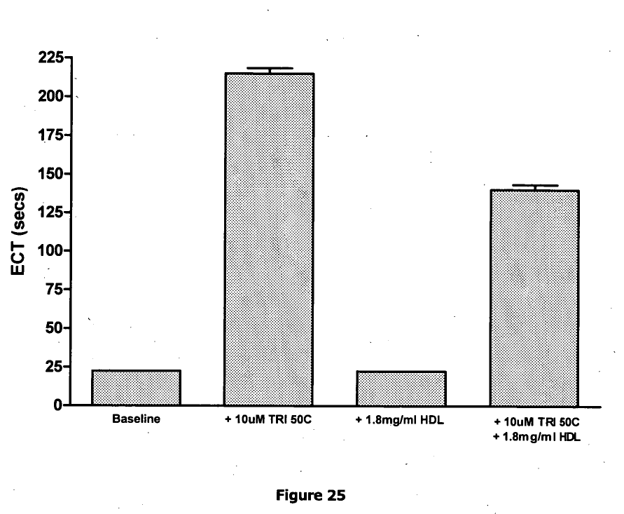

TRI 50c (10 μM) prolonged the ECT in the presence of fresh rat plasma to approximately 215 seconds. Oxidised HDL produced 40% reversible of TRI 50c activity at 10 μM (FIG. 25).

9. Oxidised HDL Stability Study

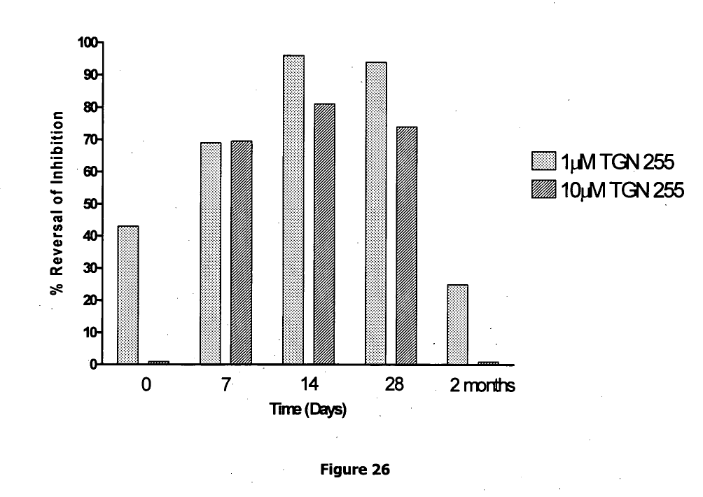

Oxidising HDL produced 43% reversible of TRI 50c (1 μM) inhibition on Day 0 (FIG. 26). Maximum neutralizing activity of oxidised HDL occurred on days 14 and 28 (96 and 94% respectively). At two months the neutralised activity declined to 25%.

In contrast, oxidizing HDL displayed no neutralizing activity at day 0 using TRI 50c at 10 μM. Maximum neutralizing activity of oxidised HDL occurred on day 14 (81%) using 10 μM TRI 50c. At two months the oxidised HDL displayed no neutralizing activity.

Conclusions

This study evaluated the ability of oxidised HDL to neutralise TRI 50c induced thrombin inhibition by using the thrombin and ecarin clotting time assays. The optimal parameters required for oxidation of HDL are: CuSO4 (500 μM) for 2.5 hr at 37° C. The oxidized HDL can reverse the inhibitory effects of TRI 50c at concentrations ranging from 1-10 μM. In contrast, non-oxidised HDL shows no neutralising activity.

The study has also demonstrated that oxidised HDL can reverse the TRI 50c ECT prolongation in both human and rat plasma. The oxidised HDL stability studies have revealed that neutralising activity can be maintained for up 14 days post-oxidation with CuSO4. The reduction of pharmacodynamic activity of TRI 50c, as measured by ECT, in the presence of oxidised HDL demonstrates that this neutralising agent may exhibit a similar profile in vivo.

Preparation of Standards

Samples of TGN 255 (14 mg/ml in methanol) and Impurity I (5.6 mg/ml in methanol) were prepared as standards. The oxidised HDL assay sample was allowed to stand at room temperature overnight and analysed the following day.

### IN VIVO EXAMPLES

The following examples relate to in vivo tests carried out to analyse the antidote effect of the compounds of the present invention towards the target active ingredient.

### Example 11

Efficacy of an Inhibitor to Neutralise the Activity of the Direct Thrombin Inhibitor TRI 50B

The efficacy of a new specific antidote towards the dynamic activity of TRI 50b was evaluated in a rat study (n=15) (Male and Female rats circa 250-300 g).

Male and female rats were anaesthetised and arterial blood sampling lines placed.

The antidote was prepared from a bovine lipoprotein following oxidation, ultrafiltration and Concentration.

The study was divided into three groups as follows:
TRI 50b alone at a dose of 2.0 mg/kg intravenously (0.8 ml/kg: Prepared in 25% ethanol/saline)
Antidote alone (5.0 ml/kg)
TRI 50b followed by administration of the antidote.

Blood samples were taken from an arterial line into 3.8% tri-sodium citrate and plasma prepared by centrifugation.

Dynamic activity of TRI 50b was assessed in plasma samples by measurement of the thrombin clotting time.

The table below shows the basic study design.

| ANIMALS/TREATMENT | | DOSE | |
|---|---|---|---|
| | Animal | | Dose |
| | numbers | | Volume |
| Group No & Description | (n) | Dose (mg/kg) | (mL/kg) |
| 1. TRI50b Controls | 2 Male | 2.0 mg/kg iv | 0.8 ml/kg |
| | 2 Female | over circa 10-15 | |
| | | seconds | |
| 2. Lipoprotein Inhibitor | 2 Male | Lipoprotein | 5.0 mL/kg |
| Controls | 2 Female | High dose | |
| 3. TRI 50B followed by | 2 Male | 2.0 mg/kg of | |
| Lipoprotein given at 3.0 | 2 Female | TRI 50b | |
| minutes post dose of TRI | | Lipoprotein | |
| 50B | | High dose | |

TRI-50b Neutralising Protein

200 ml of Lipocell (Lipoprotein fraction, Intergen catalogue number 4505-01, Lot number WT18002) was incubated at 37° C. for 37 hours. The Lipocell was then concentrated by centrifugation. The sample contained in an Amicon Centriprep YM-3 centrifugal concentrator was spun at 3800 rpm in an IEC Centra 3C centrifuge for 30 minutes at RT. The concentrated samples were pooled and centrifuged twice more to a final volume of around 50 mls. The sample was stored at 4° C.

Results

Group 1

TRI 50b alone elevated the thrombin clotting by 5.6 times above base line (23 seconds to 128 seconds) at two minutes post dose. Demonstrable anti-thrombin activity was present up to 30 minutes post dose (TT 36 seconds). The thrombin clotting time had returned to base line within 60 minutes post dose of TRI 50b.

Group 2

The antidote alone was well tolerated and without any dynamic affect as determined by measurement of thrombin clotting time up to 120 minutes post dose.

Group 3

In a third group of animals TRI 50b (2.0 mg/kg) elevated thrombin clotting time, again by 5.6 times base line (24.7 seconds to 138 seconds). Administration of the antidote at this point produced an immediate return to base line thrombin clotting values (26 seconds) which was maintained for the duration of the study (two hours). There was no evidence of any short term rebound effect as determined from thrombin clotting time measurements.

The specific lipoprotein based antidote to TRI 50b was well tolerated in a rat study without any evidence of intrinsic dynamic activity. TRI 50b (2.0 mg/kg intravenously) produced a 5.6 elevation in thrombin clotting time which was completely neutralised by the antidote (5.0 ml/kg). The activity of the antidote was maintained for the duration of the study (2 hours) without any evidence of any rebound effect.

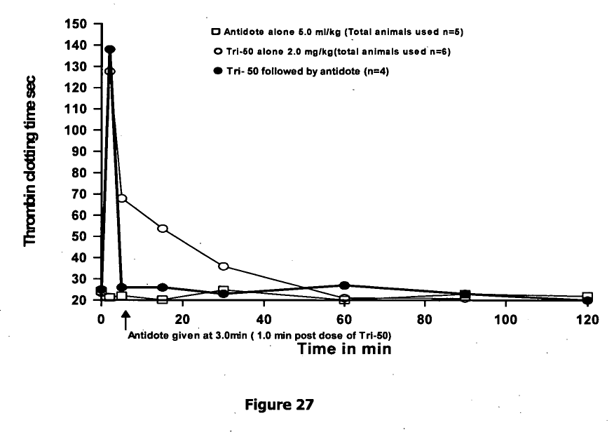

The results are shown in FIG. 27.

### Example 12

The Utility of Oxidised High Density Lipoprotein to Neutralise the Anti-Coagulant Activity of a Direct Thrombin Inhibitor, TGN 255

The objective of this study was to evaluate a range of oxidised high density lipoprotein (ox-HDL) concentrations to neutralise the anti-coagulant activity of TGN 255 in rats.

Method

Groups of male Sprague-Dawley rats were anaesthetised by gaseous isoflurane. Each animal received a bolus intravenous administration of TGN 255, using a constant dose volume of 2.5 mL/kg, followed immediately by a 30-minute intravenous infusion of TGN 255, using a constant dose volume of 10 mL/kg/hr. Immediately, the infusion of TGN 255 was discontinued, ox-HDL, saline or HDL vehicle control were administered intravenously, using a constant dose volume of 7.5 mL/kg.

Treatment groups employed for this study were as follows:

| | | Treatment 1 | | Treatment 2 | |
|---|---|---|---|---|---|
| | | dose level | | dose level | Blood sample at time (min) |
| Group | Treatment 1 | (mg/kg) | Treatment 2 | (mg/kg) | post-treatment 1 |
| 1 | TGN 255 | 2.5 (bolus) | Saline | — | 0, 15, 30, nominally 32, 40 & |
| | control | 12 (infusion) | | | 60 |
| 2 | TGN 255 | 2.5 (bolus) | Ox-HDL | 150 | 0, 15, 30, nominally 32, 40 & |
| | | 12 (infusion) | | | 60 |
| 3 | TGN 255 | 2.5 (bolus) | HDL | — | 0, 15, 30, nominally 32, 40 & |
| | | 12 (infusion) | Vehicle | | 60 |
| | | | Control | | |
| 4 | TGN 255 | 2.5 (bolus) | Non Ox- | 150 | 0, 15, 30, nominally 32, 40 & |
| | | 12 (infusion) | HDL | | 60 |
| 5 | TGN 255 | 2.5 (bolus) | Ox-HDL | 30 | 0, 15, 30, nominally 32, 40 & |
| | | 12 (infusion) | | | 60 |
| 6 | TGN 255 | 2.5 (bolus) | Ox-HDL | 10 | 0, 15, 30, nominally 32, 40 & |
| | | 12 (infusion) | | | 60 |

TGN 255 (2.5 mg/kg) was administered intravenously via the jugular vein, using a constant dose volume of 2.5 mL/kg, over approximately 10-15 seconds. This was followed by a 30-minute intravenous infusion of TGN 255 (12 mg/kg/hr) via the jugular vein, using constant dose volume of 10 mL/kg/hr.

Saline, ox-HDL, non-ox-HDL or HDL vehicle control were administered intravenously, via a tail vein immediately the infusion of TGN 255 was discontinued, using a constant dose volume of 7.5 mL/kg, over approximately 10-15 seconds.

Blood samples (approximately 0.5 mL) were taken pre-dose, at 15 and 30 minutes during TGN 255 infusion, at 2 minutes post-Treatment 2, and again at 40 and 60 minutes post the start of TGN 255 infusion. Blood was collected from the carotid artery into tri sodium citrate tubes and analysed for thrombin clotting time (TT).

Intravenous administration of the antidote, oxidised HDL, at dose levels of 10, 30 or 150 mg/kg, produced a dose-related neutralising effect on the anti-coagulant activity of TGN 255 in the rat. The highest dose of oxidised HDL (150 mg/kg) produced a 77% reduction of thrombin time. The neutralising activity of oxidised HDL was still apparent following administration of ox-HDL at 30 mg/kg, with a decrease of 56% whilst 10 mg/kg oxidised HDL produced a decrease of 40%. In the saline and HDL vehicle controls there was a 48 and 41% decline in TT respectively. The intravenous administration of the HDL vehicle control and non-oxidised HDL (150 mg/kg) had no neutralising effect in this study.

When not in use, TGN 255 was stored in a sealed container, refrigerated (nominally 2-8° C.) and protected from light.

When not in use, the HDL (oxidised and non-oxidised) was stored in sealed containers and refrigerated (nominally 4° C.).

When not in use, the HDL vehicle control was stored in sealed containers and refrigerated (nominally 4° C.).

Preparation

TGN 255 was dissolved in a nominated volume of 0.9% (w/v) saline (batch number: 05B07BD, supplied by Baxter Healthcare Ltd., Norfolk, UK) to provide a stock solution of 5.0 mg/mL.

The HDL (oxidised and non-oxidised) was supplied as a stock solution (20 mg/mL)

The vehicle control (HDL buffer) contained EDTA (0.01%), NaCl (150 mM) and CuSO₄ (500 μM). This vehicle control was supplied ready formulated by the Sponsor for the study.

The diluent for HDL was utilised to dilute the oxidised HDL stock (20 mg/mL) to obtain lower doses of oxidised HDL. Lower doses were prepared fresh from the oxidised HDL stock solution on each day of dosing as required. The diluent contained EDTA (0.01%) and NaCl (150 mM).

TGN 255 was dissolved in a nominated volume of 0.9% (w/v) saline to provide a stock solution of 5.0 mg/mL. The pH was adjusted to 7.2±0.2, using 1M HCl (batch number OC412721, supplied by BDH Laboratories Ltd., Poole, Dorset, UK). Lower doses were obtained by serial dilution of the highest concentration using 0.9% (w/v) saline.

Results

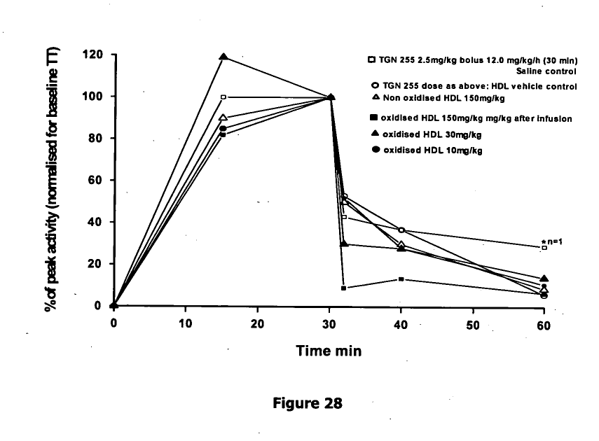

A graphical representation is shown in FIG. 28.

The group mean pre-dose thrombin time in the anaesthetised rat ranged from 22.3 to 23.2 s in this study.

The group mean thrombin time following an initial bolus administration of TGN 255 (2.5 mg/kg) and 30 minutes of intravenous infusion of TGN 255 (12 mg/kg/hr) ranged from 108.4 to 191.0 s.

For Group 1 and 3, on cessation of TGN 255 infusion, a bolus intravenous dose of saline (Group 1) or HDL vehicle control (Group 3) was administered in place of an antidote. A 2-minute post-saline/HDL vehicle control thrombin time measurements produced a decrease of 48 and 41%, respectively when compared to the thrombin time following 30 minutes of TGN 255 infusion.

Intravenous administration of the non-oxidised HDL (150 mg/kg) at the end of the TGN 255 infusion period (Group 4), produced a 42% decrease in thrombin time at 2 minutes post-administration.

Intravenous administration of the antidote, oxidised HDL (150 mg/kg) at the end of the TGN 255 infusion period (Group 2), produced a marked neutralising effect, with a 77% decrease in thrombin time at 2 minutes post-antidote administration. In addition, the TT value for Group 2 was notably lower at the 40-minute time-point compared to the control groups (Group 1 and 3).

Intravenous administration of the antidote, oxidised HDL (30 mg/kg) at the end of the TGN 255 infusion period (Group 5), produced a slight neutralising effect, with a 56% decrease in thrombin time at 2 minutes post-antidote administration.

Intravenous administration of the antidote, oxidised HDL (10 mg/kg) at the end of the TGN 255 infusion period (Group 6), produced a 40% decrease in thrombin time at 2 minutes post-antidote administration, this was comparable to the decreases seen in the vehicle control groups.

Conclusion

Intravenous administration of HDL vehicle control had no effect on the TT compared to saline-treated group at 2 minutes post-administration. The observed decline in TT with saline and HDL vehicle corresponds to the expected clearance profile of TGN 255 from the plasma with time. Similarly, the non-oxidised HDL at 150 mg/kg produced no effect on TT profile compared to both saline and HDL vehicle control. In contrast, oxidised HDL (10, 30 and 150 mg/kg) produced a 40, 56 and 77% decline in TT at 2 minutes post-administration respectively. Furthermore, the rapid decline in TT induced by ox-HDL was maintained until termination of the experiment.

These findings demonstrate that the antidote, ox-HDL is able to neutralise the anti-coagulant activity of TGN 255 in a dose dependent manner.

### Example 13

Neutralisation of High Dose TGN 255 and Determination of any Potential ‘Rebound’

The objective of this study was to evaluate: a) neutralising activity of oxidised high density lipoprotein (ox-HDL) using a high dose regimen of TGN 255 and b) investigation of a re-bound effect post ox-HDL administration in rats.

The study was conducted in two parts. Part I was designed to investigate the pharmacodynamic profile of high dose TGN 255 and the neutralising effects of oxidised HDL using anaesthetised rats. Part II of the study was designed to evaluate re-bound effects of high dose TGN 255 after neutralising with oxidised HDL in conscious rats.

Method

In Part I of this study, groups of male Sprague-Dawley rats were anaesthetised by gaseous isoflurane. Each animal received a bolus intravenous administration of TGN 255, followed immediately by a 30-minute intravenous infusion of TGN 255, according to the table below.

Immediately the infusion of TGN 255 was discontinued, oxidised HDL, or vehicle control were administered intravenously, using a constant dose volume of 7.5 mL/kg to all animals as appropriate.

Treatment groups employed for part I of this study were as follows:

| | | | | Treatment 2 | Blood sample |
|---|---|---|---|---|---|
| | | | | dose | at time |
| | | Treatment 1 | | level | (min) |
| Group | Treatment 1 | dose level (mg/kg) | Treatment 2 | (mg/kg) | post-Treatment 1 |
| 1 | TGN 255 | 5 (bolus) | Vehicle | — | 0, 15, 30, nominally 32*, |
| | | 20 (infusion) | control | | 40 & 60 |
| 2 | TGN 255 | 5 (bolus) | Oxidised | 150 | 0, 15, 30, nominally 32*, |
| | | 20 (infusion) | HDL | | 40 & 60 |
| 3 | TGN 255 | 10 (bolus) | Vehicle | — | 0, 15, 30, nominally 32*, |
| | | 30 (infusion) | control | | 40 & 60 |
| 4 | TGN 255 | 10 (bolus) | Oxidised | 150 | 0, 15, 30, nominally 32*, |
| | | 30 (infusion) | HDL | | 40 & 60 |

Two minutes following administration of Treatment 2.
TGN 255 was administered using a bolus dose volume of 1 mL/kg and an infusion dose volume of 4 mL/kg/hr (Group 1 and 2), or a bolus dose volume of 2 mL/kg and an infusion dose volume of 6 mL/kg/hr (Group 3 and 4).

Animals in Group 1 and 2 received TGN 255 (5 mg/kg, bolus) intravenously via the jugular vein, using a constant dose volume of 1 mL/kg, over approximately 10-15 seconds. This was followed by a 30-minute intravenous infusion of TGN 255 (20 mg/kg/hr) via the jugular vein, using constant dose volume of 4 mL/kg/hr.

Animals in Group 3 and 4 received TGN 255 (10 mg/kg, bolus) intravenously via the jugular vein, using a constant dose volume of 2 mL/kg, over approximately 10-15 seconds. This was followed by a 30-minute intravenous infusion of TGN 255 (30 mg/kg/hr) via the jugular vein, using constant dose volume of 6 mL/kg/hr.

All animals received oxidised HDL or vehicle control intravenously, via a tail vein immediately the infusion of TGN 255 was discontinued, using a constant dose volume of 7.5 mL/kg, over approximately 10-15 seconds.

Blood samples were taken pre-dose, at 15 and 30 minutes during TGN 255 infusion, at 2 minutes post-Treatment 2, and again at 40 and 60 minutes post the start of TGN 255 infusion. Blood was collected from the carotid artery into tri sodium citrate tubes and analysed for thrombin clotting time (TT).

In Part II of the study, groups of male Sprague-Dawley rats were individually placed in a restraining device and received a bolus intravenous injection of TGN 255 via a tail vein, followed immediately by a 30-minute infusion of TGN 255. Immediately the infusion of TGN 255 was discontinued, oxidised HDL or vehicle control was administered intravenously, via a tail vein using a constant dose volume of 7.5 mL/kg.

On the day of the study each rat was placed in the restraining device and received TGN 255 at a dose level of 10 mg/kg, as a bolus intravenous injection (administered over approximately 10-15 seconds) via a tail vein, using a constant dose volume of 2 mL/kg. Immediately following the bolus injection, each rat received TGN 255 at a dose level of 30 mg/kg/hr, by intravenous infusion, via a tail vein, using a constant dose volume of 6 mL/kg/hr. Infusion continued for 30 minutes.

Oxidised HDL or vehicle control was administered intravenously, via a tail vein immediately the infusion of TGN 255 was discontinued, using a constant dose volume of 7.5 mL/kg, over approximately 10-15 seconds. Post administration of ox-HDL or vehicle control the animals were returned to their cages until required for blood sampling.

Treatment groups employed for Part II of this study were as follows:

| | | | | Treatment 2 | Time of blood |
|---|---|---|---|---|---|
| | | Treatment 1 | | dose level | sample (min) post- |
| Group | Treatment 1 | dose level (mg/kg) | Treatment 2 | (mg/kg) | treatment |
| 1a | TGN 255 | 10 (bolus) | Vehicle control | — | 30, 60 and 240 |
| | | 30 (infusion) | | | |
| 1b | TGN 255 | 10 (bolus) | Vehicle control | — | 32* and 120 |
| | | 30 (infusion) | | | |
| 2a | TGN 255 | 10 (bolus) | Oxidised HDL | 150 | 30, 60 and 240 |
| | | 30 (infusion) | | | |
| 2b | TGN 255 | 10 (bolus) | Oxidised HDL | 150 | 32* and 120 |
| | | 30 (infusion) | | | |
| 2c | TGN 255 | 10 (bolus) | Oxidised HDL | 150 | 32* and 60 |
| | | 30 (infusion) | | | |

*Two minutes following administration of Treatment 2.
TGN 255 was administered using a bolus dose volume of 2 mL/kg and an infusion dose volume of 6 mL/kg/hr.

At the appropriate time post-dose, 0.5 mL of rat blood was collected via a tail vein (other than the one used for the administration of test article). Blood was collected into tri-sodium citrate tubes and analysed for thrombin clotting time.

In preliminary studies, TGN 255 was utilised at 5 mg/kg bolus plus 20 mg/kg/hr infusion for 30 minutes. However, thrombin time (TT) data revealed that dosing regimen did not elevate TT sufficiently to a target value of approximately >300 s which would be indicative of ‘high dose’ regimen of TGN 255. Consequently, TGN 255 dosing regimen was increased to 10 mg/kg bolus and 30 mg/kg/hr infusion.

Preparation

The vehicle control (HDL buffer) contained EDTA (0.01%), NaCl (150 mM) and CuSO₄ (500 μM).

TGN 255 was dissolved in a nominated volume of 0.9% (w/v) saline to provide a stock solution of 5.0 mg/mL. The pH was adjusted to 7.4±0.2, using 1M HCl (batch number OC412721, supplied by BDH Laboratories Ltd., Poole, Dorset, UK).

Results

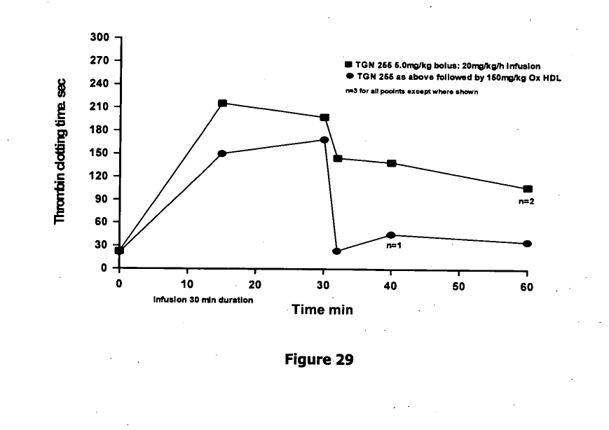

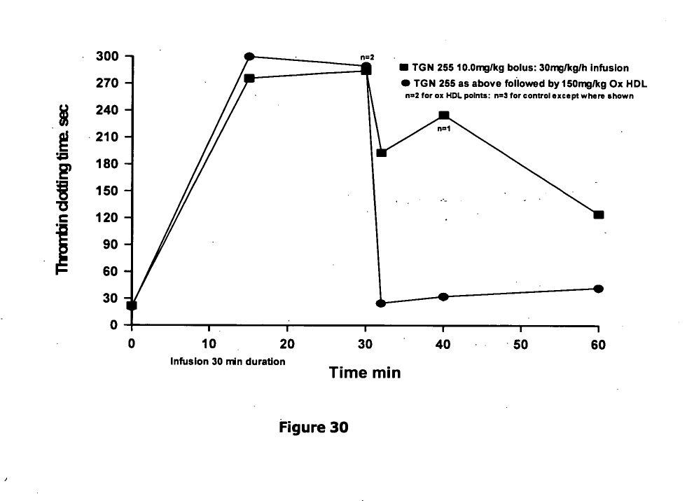

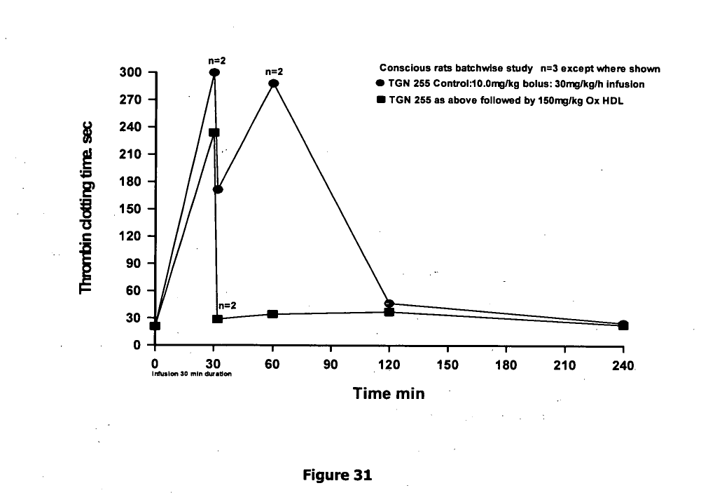

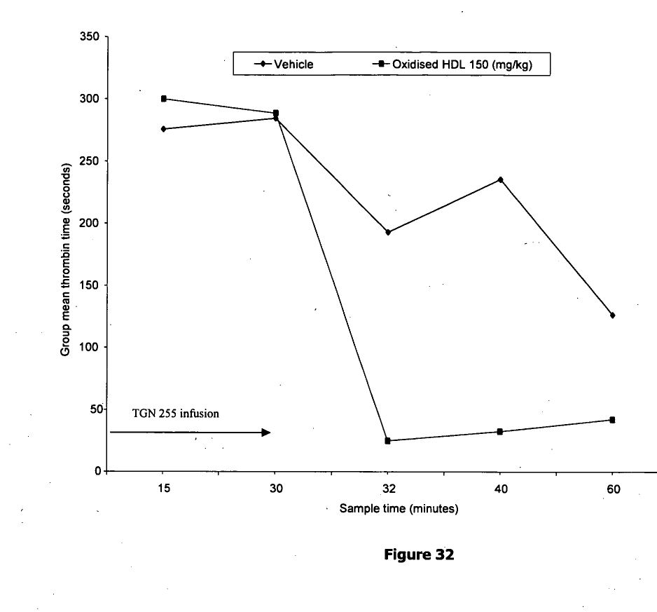

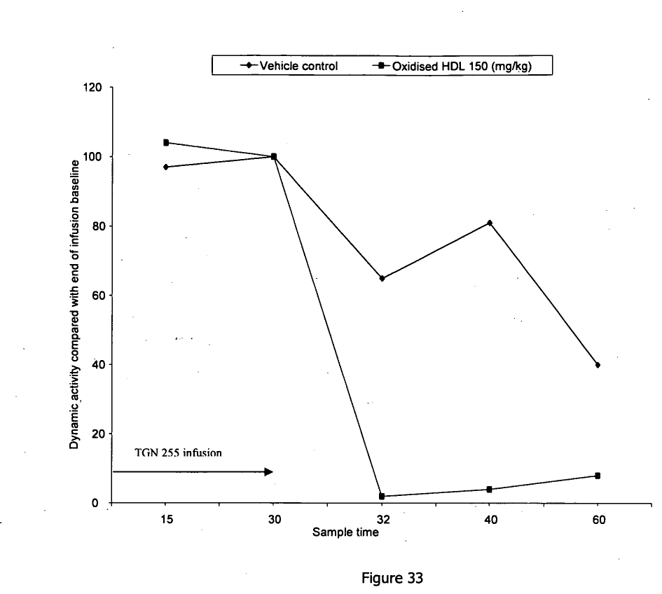

A graphical representation is shown in FIGS. 29 to 34.

Intravenous administration of vehicle control had no effect on the TT at 2 minutes post-administration. The observed decline in TT with vehicle corresponds to the expected clearance profile of TGN 255 from the plasma with time. Oxidised HDL (150 mg/kg) produced an 88% (conscious rats) and 91% (anaesthetised rats) decline in TT at 2 minutes post-administration. Furthermore, the rapid decline in TT induced by oxidised HDL was maintained until termination of the experiments.

The mean pre-dose thrombin time ranged from 20.5 to 22.8 s in this study.

Initially, rats were dosed with an intravenous bolus of TGN 255 at a dose of 5 mg/kg, immediately followed by intravenous infusion of TGN 255 at a dose of 20 mg/kg/hr. Thrombin time at the end of the infusion period was lower compared to previous studies (117.4 to 250.8 s) and as such was considered unacceptable for the purpose of this study. Additional groups were included in Part I of the study, with all rats receiving an intravenous bolus of TGN 255 at a dose of 10 mg/kg, immediately followed by intravenous infusion of TGN 255 at a dose of 30 mg/kg/hr. These doses produced thrombin times in the range of 269.4 to >300 s and this data was considered acceptable to meet the objectives of the study.

Groups 1 and 2 (TGN 255 Bolus (5 mg/kg) Followed by Infusion (20 mg/kg/hr))

The mean thrombin time for Group 1 and 2 following an initial bolus administration of TGN 255 (5 mg/kg) and 30 minutes of intravenous infusion of TGN 255 (20 mg/kg/hr) ranged from 166.2 to 197.7 s.

For Group 1, on cessation of TGN 255 infusion, a bolus intravenous dose of vehicle control was administered. A 2-minute post-vehicle control thrombin time measurement produced a decrease of 27%, when compared to the thrombin time following 30 minutes of TGN 255 infusion. In Group 2, intravenous administration of the ox-HDL (150 mg/kg) at the end of the TGN 255 infusion period produced a marked neutralising effect, with a 83% decrease in thrombin time at 2 minutes post-antidote administration.

Groups 3 and 4 (TGN 255 Bolus (10 ma/kg) Followed by Infusion (30 mg/kg/hr))

The mean thrombin time following an initial bolus administration of TGN 255 (10 mg/kg) and 30 minutes of intravenous infusion of TGN 255 (30 mg/kg/hr) ranged from 284.7 to >288.8 s.

For Group 3, on cessation of TGN 255 infusion, a bolus intravenous dose of vehicle control was administered. A 2-minute post vehicle control thrombin time measurement produced a decrease of 32%, when compared to the thrombin time following 30 minutes of TGN 255 infusion. In Group 4, intravenous administration of the ox-HDL (150 mg/kg) at the end of the TGN 255 infusion period produced a marked neutralising effect, with a 91% decrease in thrombin time at 2 minutes post-antidote administration.

Part II Results (Conscious Rat)

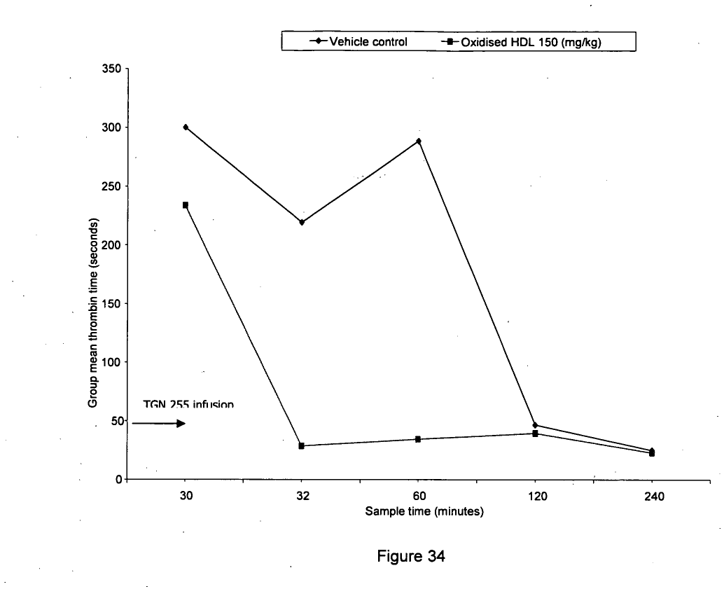

A graphical representation is shown in FIG. 34.

For Group 1, on cessation of TGN 255 infusion, a bolus intravenous dose of vehicle control was administered. A 2-minute post vehicle control thrombin time measurements produced a decrease of 27%, when compared to the thrombin time following 30 minutes of TGN 255 infusion. In Group 2, intravenous administration of the ox-HDL (150 mg/kg) at the end of the TGN 255 infusion period produced a marked neutralising effect, with a 88% decrease in thrombin time at 2 minutes post-antidote administration.

The rapid decline in thrombin time remained at a comparable level up to and including 3.5 hours following administration of ox-HDL and showed no indication of a ‘rebound’ effect in this study.

These findings demonstrate that the antidote, oxidised HDL, is able to neutralise the anti-coagulant activity of TGN 255 in an ‘over-dose’ situation with no sign of rebound up to and including 3.5 hours following administration.

### Example 14

Groups of male Sprague-Dawley rats were anaesthetised by gaseous isoflurane. Each animal received a bolus intravenous administration of TGN 255, using a constant dose volume of 2.5 mL/kg, followed immediately by a 30-minute intravenous infusion of TGN 255, using a constant dose volume of 10 mL/kg. Immediately the infusion of TGN 255 was discontinued, antidotes or saline were administered intravenously, using a constant dose volume of 7.5 mL/kg.

Treatment groups employed for this study were as follows:

| | | Treatment 1 | | Treatment 2 | Blood sample at time |
|---|---|---|---|---|---|
| | Treatment | dose level | | dose level | (min) post the start of |
| Group | 1 | (mg/kg) | Treatment 2 | (mg/kg) | Treatment 1 |
| 1 | TGN 255 | 2.5 (bolus) | Saline | — | 0, 15, 30, nominally 32, 40 |
| | control | 12 (infusion) | | | & 60 |
| 2 | TGN 255 | 2.5 (bolus) | HDL | 150 | 0, 15, 30, nominally 32, 40 |
| | | 12 (infusion) | | | & 60 |

The antidote HDL (20 mg/ml) was supplied in a buffer solution ready for use in this study.

TGN 255 was dissolved in a nominated volume of 0.9% (w/v) saline to provide a stock solution of 5.0 mg/mL. The pH was adjusted between 7.33-7.41 using 1M HCl (batch number OC346964, supplied by BDH Laboratories Ltd., Poole, Dorset, UK). Lower doses were obtained by serial dilution of the highest concentration using 0.9% (w/v) saline.

All formulations were discarded within 10 hours of preparation.

TGN 255 (2.5 mg/kg) was administered intravenously via the jugular vein, using a constant dose volume of 2.5 mL/kg, over approximately 10-15 seconds. This was followed by a 30-minute intravenous infusion of TGN 255 (12 mg/kg/hr) via the jugular vein, using constant dose volume of 10 mL/kg/hr.

Antidotes or saline were administered intravenously, via a tail vein immediately the infusion of TGN 255 was discontinued, using a constant dose volume of 7.5 mL/kg, over approximately 10-15 seconds.

Blood samples (approximately 0.5 mL) were taken pre-dose, at 15 and 30 minutes during TGN 255 infusion, at 2 minutes post-Treatment 2, and again at 40 and 60 minutes post the start of TGN 255 infusion. Blood was collected from the carotid artery into tri sodium citrate tubes and analysed for thrombin clotting time (TT).

Intravenous administration of the antidote HDL (150 mg/kg) to the anaesthetised rat, produced a marked neutralising effect on the anti-coagulant activity of TGN 255 in this study.

Results

The results are summarised in Table 1.

The group mean pre-dose thrombin time in the anaesthetised rat ranged from 20.7 to 21.4 s in this study.

The group mean thrombin time following an initial bolus administration of TGN 255 (2.5 mg/kg) and 30 minutes of intravenous infusion of TGN 255 (12 mg/kg/hr) ranged from 124.4 to 182.7 s.

For Group 1, on cessation of TGN 255 infusion, a bolus intravenous dose of saline was administered in place of an antidote. A 2-minute post-saline thrombin time measurement showed there to be a decrease of 35% when compared to the thrombin time following 30 minutes of TGN 255 infusion.

Administration of the antidote HDL (150 mg/kg) at the end of the TGN 255 infusion period (group 2), produced a 80% decrease in thrombin time at 2 minutes post-antidote administration. In addition, TT values for group 2 were substantially lower at 32 and 40 minutes time points compared to groups 1 and 3.

Intravenous administration of the antidote, HDL (150 mg/kg) to the rat produced a marked neutralising effect on the anti-coagulant activity of TGN 255 in this study.

TABLE 1
Effects of intravenously administered antidotes on group mean thrombin time (TT) in the anaesthetised rat

| | | | Group | | | | | |
|---|---|---|---|---|---|---|---|---|
| | Treatment 1 & | Treatment 2 | mean TT | Group mean TT (s ± sd) at time (min) | | | | |
| | dose level | & dose level | (s ± sd) | post the start of TGN 255 infusion | | | | |
| Group | (mg/kg) | (mg/kg) | pre-dose | 15 | 30 | 32 | 40 | 60 |
| 1 | TGN 255 control | Saline - | 21.4⁽⁶⁾ ± 1.21 | 162.7⁽⁶⁾ ± 30.14 | 153.9⁽⁵⁾ ± 37.62 | 99.8⁽⁵⁾ ± 36.88 | 62.6⁽⁵⁾ ± 27.07 | 54.7⁽²⁾ ± 6.43 |
| | 2.5 (bolus) | | | | | | | |
| | 12 (infusion) | | | | | | | |
| 2 | TGN 255 | HDL | 20.7⁽⁴⁾ ± 1.66 | 154.2⁽⁴⁾ ± 28.34 | 124.4 ± 77.30 | 24.9⁽⁴⁾ ± 1.65 | 27.5⁽⁴⁾ ± 3.58 | 27.5⁽⁴⁾ ± 3.59 |
| | 2.5 (bolus) | 150 | | | | | | |
| | 12 (infusion) | | | | | | | |

sd standard deviation
All groups consisted of 3 animals except where stated in ( ).
TGN 255 (12 mg/kg/hr) was infused for 30 minutes.
The nominal 32-minute post-dose sample was taken 2 minutes post administration of Treatment 2.

1. A method for therapeutically reducing or substantially destroying the activity of an organoboronate drug in a subject, comprising administering to the subject a therapeutically useful amount of a lipid in oxidised form.

2. The method of claim 1 wherein the lipid comprises an unsaturated fatty acid.

3. The method of claim 2 wherein the oxidised lipid is a hydroperoxide.

4. The method of claim 1 wherein the lipid in oxidised form has the characteristics of a product obtained by contacting the lipid with a source of Cu (II) in the presence of oxygen.

5. The method of claim 2 wherein the lipid in oxidised form has the characteristics of a product obtained by contacting the lipid with a source of Cu (II) in the presence of oxygen.

6. The method of claim 4 wherein the concentration of Cu (II) is between at least about 0.01 mM and about 5 mM.

7. The method of claim 1 wherein the lipid is in the form of a lipoprotein.

8. The method of claim 3 wherein the lipid is an LDL.

9. The method of claim 3 wherein the lipid is an HDL.

10. The method of claim 7 wherein the lipoprotein is of bovine origin.

11. The method of claim 7 wherein the lipoprotein is of human origin.

12. The method of claim 7 wherein the lipoprotein in oxidised form has the characteristics of a product obtained by contacting the lipoprotein with a source of Cu (II) is in a dilution in the range of 1 in 3 up to 1 in 81 in an aqueous medium.

13. The method of claim 12 wherein the dilution is in the range of 1 in 5 to 1 in 20.

14. The method of claim 1 wherein the boronate group (—B(OH)₂) of the organoboronate drug is bonded to an aliphatic carbon atom.

15. The method of claim 3 wherein the boronate group (—B(OH)₂) of the organoboronate drug is bonded to an sp³ carbon atom.

16. The method of claim 3 wherein the organoboronate drug is a peptide boronate.

17. The method of claim 16 wherein the peptide boronate has a C-terminal residue which is of an α-aminoboronic acid having an alkyl or alkoxyalkyl side chain.

18. The method of claim 17 wherein the C-terminal residue is of Boro-3-methoxypropylglycine.

19. The method of claim 3 wherein the drug is Cbz-(R)-Phe-(S)-Pro-(R)-Mpg-B(OH)₂, whether administered as the free acid, a salt or a prodrug, Mpg-B(OH)₂ being a residue of an amino boronic acid of the formula:
H₂N—CH((CH₂)₃OMe)B(OH)₂.

20. A method for treating in a subject bleeding resulting from the administration of an aminoboronate inhibitor of a coagulation serine protease, comprising administering to the subject a therapeutically effective amount of a lipid in oxidised form as defined in claim 1.

21. The method of claim 20 wherein the oxidised lipid comprises a lipoprotein hydroperoxide and the aminoboronate is of formula (III) or is a salt or prodrug thereof: 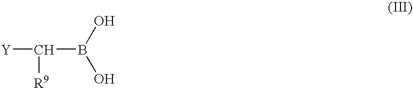
wherein
Y comprises a moiety which, together with the fragment —CH(R⁹)—B(OH)₂, has affinity for the substrate binding site of thrombin; and
R⁹ is a straight chain alkyl group interrupted by one or more ether linkages and in which the total number of oxygen and carbon atoms is 3, 4, 5 or 6 or R⁹ is —(CH₂)ₘ—W where m is 2, 3, 4 or 5 and W is —OH or halogen.

22. The method of claim 21 wherein the aminoboronate is of Formula (X) or is a salt or prodrug thereof: 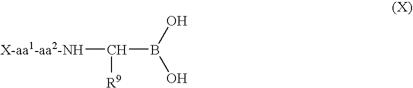
wherein
X is H (to form NH₂) or an amino-protecting group;
aa¹ is Phe, Dpa or a wholly or partially hydrogenated analogue thereof;
aa² is an imino acid having from 4 to 6 ring members; and
R¹ is a group of the formula —(CH₂)ₘ—W, where m is 2, 3 or 4 and W is —OH, —OMe, —OEt or halogen (F, Cl, Br or I).

23. The method of claim 21 wherein the aminoboronate is TRI 50c, a compound of Formula
Cbz-(R)-Phe-(S)-Pro-(R)-Mpg-B(OH)₂,
or is a salt or prodrug thereof, Mpg-B(OH)₂ being a residue of an amino boronic acid of the formula:
H₂N—CH((CH₂)₃OMe)B(OH)₂.

24. The method of claim 23 wherein the aminoboronate is a base addition salt, anhydride or anhydride salt of TRI 50c.

25. A pharmaceutical formulation comprising a therapeutically effective amount of an oxidised lipid.

26. The formulation of claim 25 wherein the lipid comprises an unsaturated fatty acid.

27. The formulation of claim 25 wherein the oxidised lipid is a lipoprotein hydroperoxide.

28. The formulation of claim 25 wherein the oxidised lipid has the characteristics of a product obtained by contacting the lipid with a source of Cu (II) in the presence of oxygen.

29. The formulation of claim 25 which is adapted for intravenous administration.

30. The formulation of claim 29 wherein the oxidised lipid is oxidised HDL.

31. The formulation of claim 25 which includes a pharmaceutically acceptable diluent, excipient or carrier.

32. A method for terminating or reducing activity of a boropeptidyl serine protease inhibitor in a subject, comprising administering to the subject an effective amount of the lipid of claim 1.

33. A method for treating bleeding resulting from the administration of a boropeptidyl serine protease inhibitor comprising the amino acid sequence Phe-Pro-BoroMpg inhibitor to a subject, comprising administering to the subject an effective amount of the lipid of claim 3.

34. The method of claim 32 wherein the lipid peroxide is a sterol ester peroxide.

35. The method of claim 34 wherein the lipid peroxide is cholesteryl ester peroxide.

36. A method of production of a pharmaceutical composition for therapeutically neutralising an organoboronate drug, comprising contacting a lipid with an oxidising agent to form a product, and further combining the product with a pharmaceutically acceptable diluent, carrier or excipient.

37. The method of claim 36, wherein the lipid is a lipoprotein and the oxidising agent comprises a source of Cu(II) ions in the presence of oxygen.

38. A method of providing a medicament pair comprising a first medicament for the treatment of thrombosis by prophylaxis or therapy and a second medicament to inhibit the action of the first medicament in the event of undue bleeding, comprising
providing a pharmaceutical composition comprising a compound which is capable of providing in the plasma a peptide boronic acid of formula (A); and
providing a pharmaceutical formulation of claim 25,
formula (A) being as follows: 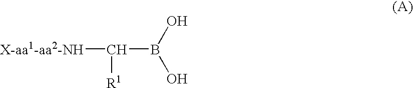
where:
X is H (to form NH₂) or an amino-protecting group;
aa¹ is Phe, Dpa or a wholly or partially hydrogenated analogue thereof;
aa² is an imino acid having from 4 to 6 ring members; and
R¹ is a group of the formula —(CH₂)ₘ—W, where m is 2, 3 or 4 and W is OH, -OMe, —OEt or halogen (F, Cl, Br or I).

39. A method for treating thrombosis by prophylaxis or therapy using a medicament which results in inappropriate bleeding and then inhibiting the action of said medicament, wherein
a therapeutically effective amount of a pharmaceutical composition comprising a compound of formula (A) as recited in claim 38 is administered to a patient in need thereof to treat thrombosis, and, after the inappropriate bleeding,
a therapeutically effective amount of an oxidised lipoprotein is administered to the patient to inhibit the pharmaceutical composition.

40. A method according to claim 39, wherein the oxidised is administered intravenously.
